(19) United States
(12) Patent Application Publication
(10) Pub. No.: US 2007/0026500 A1
(43) Pub. Date: Feb. 1, 2007

(54) Crystalline Neutrokine-alpha protein, method of preparation thereof, and method of use thereof

(76) Inventors: Yuling Li, Potomac, MD (US); Deena E. Oren, East Brunswick, NJ (US); Edward Arnold, Belle Meade, NJ (US); Yulia Volovik, North Brunswick, NJ (US)

Correspondence Address: HUMAN GENOME SCIENCES INC;INTELLECTUAL PROPERTY DEPT., 14200 SHADY GROVE ROAD, ROCKVILLE, MD, 20850, US

(21) Appl. No.: 10/839,694

(22) Filed: May 6, 2004

Related U.S. Application Data
(63) Continuation of application No. PCT/US02/35661, filed on Nov. 7, 2002.
(60) Provisional application No. 60/331,049, filed on Nov. 7, 2001.

(51) Int. Cl. C07K 14/525; G06F 19/00; G01N 33/48; G01N 33/50; C07H 21/04; C12P 21/02

(52) U.S. Cl. 435/69.5; 435/320.1; 435/325; 530/351; 536/23.5; 702/19

(57) ABSTRACT

The invention relates to a Neutrokine-alpha protein in crystalline form, a method of preparing a Neutrokine-alpha protein in crystalline form, and methods of using a Neutrokine-alpha protein in crystalline form. In particular, the three-dimensional structure of a Neutrokine-alpha protein in crystalline form is used to design molecules that have biological activity. The methods are useful for designing compounds that bind to a Neutrokine-alpha protein, inhibit a Neutrokine-alpha protein, mimic a Neutrokine-alpha protein, and/or enhance the activity of a Neutrokine-alpha protein. The three-dimensional structure of a Neutrokine-alpha protein, as provided herein, is also used to determine the three-dimensional of other Neutrokine-alpha proteins and homologues thereof.

### BACKGROUND OF THE INVENTION

1. Field of the Invention

The present invention relates to the field of tumor necrosis factors, and in particular to the characterization and use of a Neutrokine-alpha protein in crystalline form. Additionally, the present invention relates to a methods of preparing a Neutrokine-alpha protein in crystalline form, determining the three-dimensional structure of a Neutrokine-alpha protein, and designing biologically active molecules based on the three-dimensional structure of a Neutrokine-alpha protein.

2. Background Art

Human tumor necrosis factors, e.g., TNF-α and TNF-β, are related members of a broad class of polypeptide mediators, which includes the interferons, interleukins and growth factors, collectively called cytokines (Beutler, B. and Cerami, A., Annu. Rev. Immunol. 7:625-655 (1989)). Sequence analysis of cytokine receptors has defined several subfamilies of membrane proteins (1) the Ig superfamily, (2) the hematopoietin (cytokine receptor superfamily) and (3) the tumor necrosis factor (TNF)/nerve growth factor (NGF) receptor superfamily. For a review of the TNF superfamily, see Gruss and Dower, Blood 85:3378-3404 (1995) and Aggarwal and Natarajan, Eur. Cytokine Netw. 7:93-124 (1996). The TNF/NGF receptor superfamily contains at least 10 different proteins. Ligands for these receptors have been identified and belong to at least two cytokine superfamilies.

Some of the known members of the TNF-ligand superfamily include TNF-α, TNF-β (lymphotoxin-(α), LT-β, OX40L, Fas ligand, CD30L, CD27L, CD40L, and 4-IBBL. The ligands, members of the TNF ligand superfamily, are acidic, TNF-like molecules with approximately 20% sequence homology in the extracellular domains (range, 12%-36%) and exist mainly as membrane-bound forms with the biologically active form being a trimeric/multimeric complex. Soluble forms of the TNF ligand superfamily have only been identified so far for TNF-α, LT-β, and Fas ligand. For a general review, see Gruss, H. and Dower, S. K., Blood 85:3378-3404 (1995). These proteins participate in the regulation of cell proliferation, activation, and differentiation, including control of cell survival or death by apoptosis or cytotoxicity (Armitage, R. J., Curr. Opin. Immunol. 6:407 (1994) and Smith, C. A., Cell 75:959 (1994)).

An additional member of the TNF-ligand superfamily has recently been discovered. Neutrokine-alpha (also known as BLyS™ (B-Lymphocyte Stimulator); also known as TALL-1, THANK, BAFF, zTNF4, and TNSF13B) is a member of the tumor necrosis factor (TNF) superfamily that induces B cell proliferation and immunoglobulin secretion and appears to be a key regulator of peripheral B cell populations in vivo (Moore et al., Science 285:260-263 (1999); Mackay et al., J. Exp. Med 190:1697-1710(1999)). Like other members of the TNF family, Neutrokine-alpha is a type-II membrane protein that may be cleaved at the cell surface to form a soluble protein (Mariani et al., J. Cell Biol. 137:221-229 (1997)). The crystal structures of a number of TNF ligands have been determined (Eck et al., J. Biol. Chem. 267:2119-2122 (1992); Eck et al., J. Biol. Chem. 264:17595-17605 (1989); Hymowitz et al., Biochemistry 39:633-640 (2000); Cha et al., J. Biol. Chem. 275:31171-31177 (2000); Lam et al., J. Clin. Invest. 108:971-979 (2001)), two in complex with the respective receptors [Banner et al., Cell 73:431-445 (1993); Cha et al., J. Biol. Chem. 275:31171-31177 (2000); Singh et al., Protein Sci. 7:1124-1135 (1998); Mongkolsapaya et al., Nat. Struct. Biol. 6:1048-1053 (1999)). While the TNF ligand family shows significant sequence diversity, members are closely related in terms of their structures. All ligands described so far are active as trimers, and Neutrokine-alpha has activity as a trimer as well.

Like other members of the TNF family, Neutrokine-alpha is a ligand that interacts with several receptors. Neutrokine-alpha was initially shown to interact with TACI (trans-membrane activator and CAML interactor) and BCMA (B cell maturation antigen) (Gross et al., Nature 404:995-999 (2000)). Both receptors were found to bind APRIL as well [Marsters et al., Curr. Biol. 10:785-788 (2000); Wu et al., J. Biol. Chem. 275:35478-35485 (2000)), APRIL being the TNF-like ligand that has the highest degree of sequence homology with Neutrokine-alpha. Most recently, a third receptor, termed BAFF-R, has been identified. This receptor apparently does not interact with APRIL or any TNF-like ligand other than Neutrokine-alpha (Thompson et al., Science 293:2108-2111 (2001)). Experiments using transgenic animals have shown that the interaction of Neutrokine-alpha with TACI and BCMA plays a role in the development of autoimmune disease (Gross et al., Nature 404:995-999 (2000)). At the same time, Neutrokine-alpha is a crucial factor for the normal development of B cells, and apparently this function is mediated through a BCMA-independent pathway (Schiemann et al., Science 293:2111-2114 (2001).

The biological actions of Neutrokine-alpha suggest several potential therapies in which the action of Neutrokine-alpha is mimicked or enhanced. For example, common variable immunodeficiency (CVID) is a group of immunodeficiency syndromes in which B cell immunity is abnormal. Most patients have normal or near-normal numbers of circulating B cells, but the cells fail to differentiate into effective plasma B cells. As a result, patients have low or undetectable amounts of serum antibodies. The condition may result from insufficient stimulation of B cells rather than from a failure intrinsic to B cells (Rosen et al., New Eng. J. Med. 333:7 (1995)). Most patients with CVID experience acute, recurring bacterial infections, including pneumonia, bronchitis, and sinusitis (“Immune Deficiency and Allied Disorders: Clinical Updates,” Immune Deficiency Foundation Vol. II, Issue 1, July 1995). Current treatment involves regular administration of intravenous antibodies, which are prepared from pooled blood samples from thousands of individual donors. The administration of Neutrokine-alpha protein may boost antibody levels in patients with CVID, as well as in other immunodeficiency conditions that effectively mimic CVID.

Immunoglobulin-A deficiency is a disorder of the immune system characterized by increased susceptibility to infection. Patients with this disease fail to produce normal amounts of immunoglobulin-A, which provides the first line of defense for the inner surfaces of the body against infections of the lung, the intestine, the mouth, the urogenital tract, and other areas lined by mucosal membranes. It is believed that immunoglobulin-A deficiency may result from the failure of the B lymphocyte to mature into plasma cells that produce immunoglobulin-A antibodies. Symptomatic patients suffer from recurrent and serious infections, including infections of the gastrointestinal tract, lungs and sinuses, as well as allergic disorders, epilepsy, and cancer. There are currently no available therapies that address the underlying cause of immunoglobulin-A deficiency. Treatment with Neutrokine-alpha may help immunoglobulin-A deficient patients produce their own antibodies. The Neutrokine-alpha protein is known to be able to stimulate B cells to produce immunoglobulin-A antibodies as well as other types of antibodies. Preclinical studies have also shown that Neutrokine-alpha proteins can stimulate the B cells of some immunoglobulin-A deficient patients to enhance the production of immunoglobulin-A antibodies.

Several types of cancer, including chronic lymphocytic leukemia and multiple myeloma, affect the immune system's ability to fight off infections by impairing antibody production. Neutrokine-alpha may help these patients fend off infectious disease. Cancer therapies also damage the immune system. In some cases it may take years for the full antibody response to recover following cancer treatment. Treatment with Neutrokine-alpha after cancer therapy may speed recovery of a fully competent immune system.

Other uses of Neutrokine-alpha include treating patients that receive immunosuppressive drugs that make them vulnerable to infections; treating patients infected with antibiotic-resistant bacteria; use as a vaccine adjuvant; use as Neutrokine-alpha linked to radionucleotides that have potential application as therapy for B-cell malignancies such as non-Hodgkin's lymphoma, chronic lymphocytic leukemia, and multiple myeloma.

Compounds that prevent or inhibit the activity of Neutrokine-alpha also have therapeutic uses. The positive regulatory effects of Neutrokine-alpha on B cells and on T-cell-dependent humoral responses, the autoimmune phenotype of Neutrokine-alpha transgenic mice, and the high levels of Neutrokine-alpha in lupus-prone mice suggest that blocking the interaction between Neutrokine-alpha and its receptors may be a useful therapeutic approach in lupus.

Additionally, the immune system has to distinguish the body's own cells and tissues from those of pathogens so that it can avoid attacking itself while maintaining a diverse repertoire of antibodies. Abnormalities in the induction or maintenance of self-tolerance—the process that prevents the immune system from attacking the body's own tissues—can lead to inflammatory immune responses developing against self-antigens and thus to autoimmune disease. B cells that produce antibodies that recognize parts of the normal body play an important role in many autoimmune diseases. Systemic lupus erythromatosus, rheumatoid arthritis, multiple sclerosis, Crohn's disease, diabetes, and some forms of asthma are all examples of autoimmune diseases. Thus, agents that inhibit the proliferation of B cells, i.e., antagonists of Neutrokine-alpha activity, have potential to treat or prevent diseases such as systemic lupus erythromatosus, rheumatoid arthritis, multiple sclerosis, Crohn's disease, diabetes, Wegener's granulomatous, myasthenia gravis, and some forms of asthma.

Although Neutrokine-alpha may be used as an effective agent to treat some of the aforementioned conditions, there exists a need for additional, effective therapeutic agents that mimic the biological activity of Neutrokine-alpha. Moreover, there exists the need for additional, effective therapeutic agents that inhibit the biological activity of Neutrokine-alpha. The three dimensional structure of a Neutrokine-alpha protein would permit the more efficient development and design of both agonists and antagonists of Neutrokine-alpha. Additionally, the three dimensional structure of Neutrokine-alpha would allow the elucidation of the three-dimensional structures of related proteins. Moreover, computer systems comprising the three-dimensional structure of a Neutrokine-alpha protein would facilitate the preparation of biologically active molecules that are useful for the above indications.

### SUMMARY OF THE INVENTION

One aspect of the present invention is a Neutrokine-alpha protein in crystalline form. In particular, human Neutrokine-alpha protein in crystalline form is one aspect of the present invention.

An additional aspect of the present invention is a composition comprising a Neutrokine-alpha protein, wherein said composition is suitable for forming Neutrokine-alpha in crystalline form.

Another aspect of the present invention is a method of crystallizing a Neutrokine-alpha protein. The crystallized Neutrokine-alpha protein can be analyzed to provide X-ray diffraction patterns of sufficiently high resolution to be useful for determining the three-dimensional protein structure.

Another aspect of the present invention is directed to determining the three-dimensional structure of a Neutrokine-alpha protein by using X-ray diffraction crystallography methods. The X-ray diffraction patterns can be either analyzed directly to provide the three-dimensional structure (if sufficient data is collected), or atomic coordinates for the crystallized Neutrokine-alpha, as provided herein, can be used for structure determination.

An additional aspect of the present invention is a method of determining the three-dimensional structure of a Neutrokine-alpha protein by using the atomic coordinates of human Neutrokine-alpha protein in crystalline form. The atomic coordinates of human Neutrokine-alpha protein in crystalline form and the amino acid sequence of a second Neutrokine-alpha protein are entered into one or more computer programs for molecular modeling. Such molecular modeling programs generate atomic coordinates that reflect the secondary, tertiary, and/or quaternary structures of the protein which contribute to its overall three-dimensional structure and provide information related to binding and/or active sites of the second Neutrokine-alpha protein.

An additional aspect of the present invention is a method of designing a biologically active compound that enhances, mimics, inhibits, or antagonizes the activity of a Neutrokine-alpha protein. The three-dimensional structure of a Neutrokine-alpha protein is used to design said biologically active compound. Additionally, said biologically active compound is optionally synthesized and optionally assayed to test for biological activity.

Another aspect of the present invention is a computer-readable medium comprising the three-dimensional structure of a Neutrokine-alpha protein. An additional aspect of the present invention is a computer system comprising a memory and a processor, wherein said memory comprises the three-dimensional structure of a Neutrokine-alpha protein

### BRIEF DESCRIPTION OF THE FIGURES

FIG. 1 provides the sequence of soluble human Neutrokine-alpha. Also provided is a structure-based sequence alignment of human Neutrokine-alpha with other members of the cytokine family, including TNF-α, TNF-β, TRAIL, CD40L, and RANKL. FIG. 1 additionally displays a ribbon diagram of the three-dimensional structure of a monomer of human Neutrokine-alpha.

FIGS. 2A, 2B, 2C and 2D provide ribbon diagrams of three dimensional structure of trimerized human Neutrokine-alpha. FIG. 2A depicts a hydrated magnesium ion at the center of the trimer. FIG. 2B′ additionally provides a more detailed view of the bound magnesium ions along with certain amino acid residues of Neutrokine-alpha. FIG. 2E shows a portion of the electron density map determined from the X-ray diffraction data. Specifically, FIG. 2E details the region of the disulfide bond between residues 232 and 245.

FIG. 3 provides images of the three-dimensional structures, including the solvent accessible surface, of Neutrokine-alpha, TNF-α, TNF-β, TRAIL, CD40L, and RANKL. The arrows in the images point to areas on the surface of the protein, and illustrate how the structure of Neutrokine-alpha is unique among the proteins.

FIG. 4 provides the image of three-dimensional structures of TNF-β/TNF-R complex; TRAIL/DR5 complex; Neutrokine-alpha; and Neutrokine-alpha rotated 90° about the x-axis. Additionally, the residues of Neutrokine-alpha comprising the putative receptor-binding site (the “groove”) are listed. The residues of each of the receptors that are believed to comprise the binding site for cytokine ligand are listed for each of TNF-R, DR5, TNR2, BAFF-R, BCMA, and TACI.

FIG. 5 provides the results of a receptor binding study by SELDI affinity mass spectrometry. The results show that, for the interaction of Neutrokine-alpha with both recombinant BCMA and TACI receptors, the AA″ and the DE loops of the molecule are centrally involved.

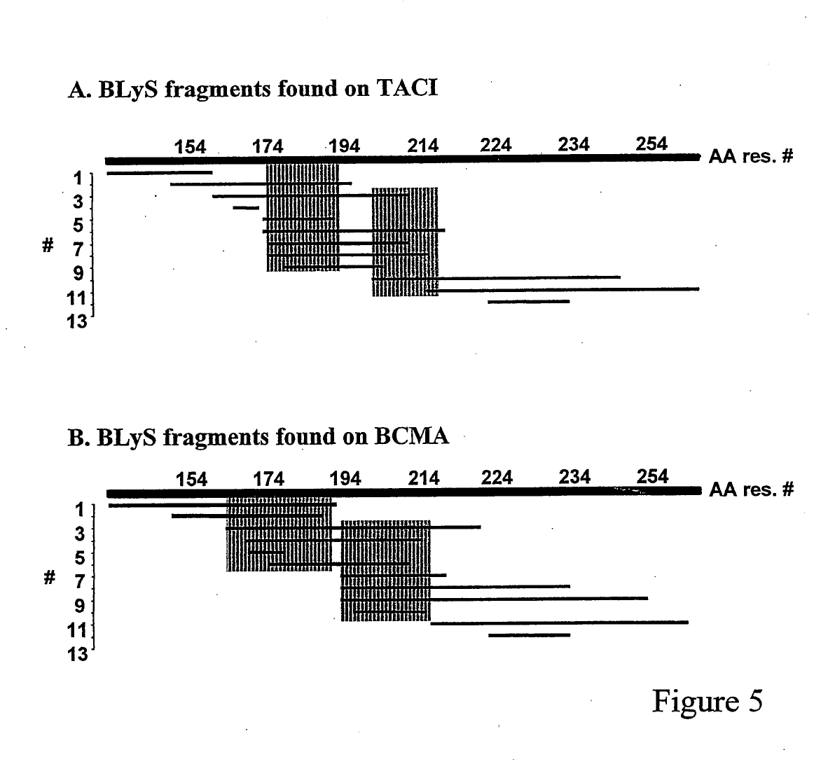

FIG. 6 provides the structure of a computer system as described herein.

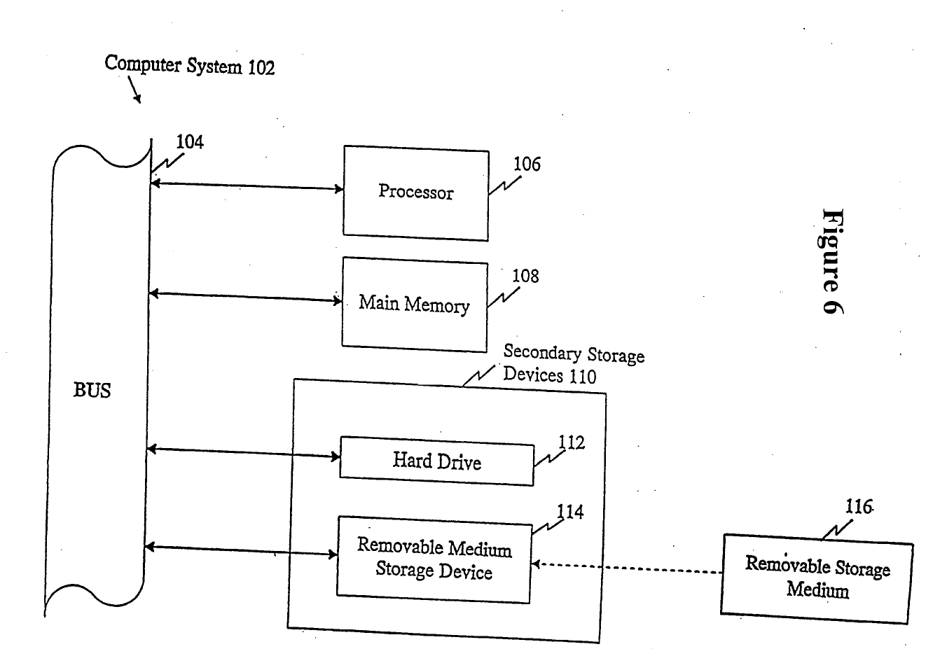

FIG. 7 provides the image of solvent accessible surface of a trimer of monomers of Neutrokine-alpha. Additionally, several of the amino acids which compose a major groove are indicated. This major groove is herein identified as a target for drug design or identification using the methods disclosed herein.

FIG. 8 provides the image of the solvent accessible surface of a trimer of monomers of hNeutrokine-alpha. The image in FIG. 8 is of the same protein structure as in FIG. 7 but from a different perspective, rotated approximately 90° along one axis. Additionally, several of the amino acids which compose grooves on the surface are indicated. These grooves are herein identified as a target for drug design or identification using the methods disclosed herein.

FIG. 9 provides the image of the solvent accessible surface of a monomer of hNeutrokine-alpha. The major portion that is visible in the image represent the surface of the monomer that participates in trimerization of monomers. Several amino acids which compose grooves on the surface are indicated. The areas identified in the figure are herein indicated as being useful for drug design or identification using the methods disclosed herein.

FIGS. 10A and 10B provide the graphical results of neutrokine-alpha/receptor interactions. 10A. Superimposed TNF-receptor peptide (TNF-R) (ribbon) docked on neutrokine-alpha surface representation, with TNF-R peptide shown binding to major surface groove. The middle image of 10A is the same but rotated 90 degrees. On the right, groove residues in common between hneutrokine-alpha and APRIL are colored in shaded. The residues forming the groove from adjacent monomers are GLN148, ILE150, ALA151, ASP152, SER153, GLU154, LEU169, LEU170, PHE172, LEU200, THR202, ILE270, SER271, LEU272, ASP273, GLU274, ASP275, and PHE278 from one monomer, and THR190, TYR192, ALA207, GLY209, HIS210, LEU211, GLN213, ARG214, LYS216, HIS218, PHE220, ASP222, GLU223, LEU224, LEU226, VAL227, THR228, LEU229, PHE230, ARG231, ILE233, ALA251, LYS252, LEU253, GLU254, and ASP257 from another monomer. Those in common with APRIL are underlined. FIG. 10B. PAWS coverage analysis, mapping fragments found in SELDI binding assays of TACI and BMCA to areas in the neutrokine-alpha sequence. Boxes highlight areas of strongest coverage. Binding site mapping was done by in situ trypsin digestion of the captured ligand, followed by mass spectrometric identification of retained fragments. Arrows indicate neutrokine-alpha beta-strands.

### DETAILED DESCRIPTION OF THE INVENTION

The present invention provides a Neutrokine-alpha protein in crystalline form. A Neutrokine-alpha protein in crystalline form has the characteristics as described herein. The space group of said Neutrokine-alpha protein in crystalline form is preferably hexagonal. The unit cell dimensions of said space group are defined by a, b, c, α, β, and γ, wherein a is from about 120 Å to about 125 Å, b is from about 120 Å to about 125 Å, and c is from about 158 Å to about 164 Å, α is from about 85 to about 95, β is from about 85 to about 95, and γ is from about 115 to about 125. Preferably, α is about 90, β is about 90, and γ is about 120.

A Neutrokine-alpha protein in crystalline form can also be characterized by crystal density measurements using Ficoll gradients (Z). According to the present invention, Z is from about 1 to about 12. Preferably, Z is about 6, indicating that there are six Neutrokine-alpha monomers per asymmetric unit. For more details regarding Ficoll gradients, see Westbrook, E. M. Methods Enzymol. 114:187-96 (1985).

A Neutrokine-alpha protein in crystalline form can also be characterized by Matthew's coefficient. For a Neutrokine-alpha protein in crystalline form according to the present invention, Matthew's coefficient is from about 2 Å³ per Dalton (Da) to about 5 Å³ per Da. Preferably, Matthew's coefficient is from about 3 Å³ per Da to about 4 Å³ per Da. Preferably, Matthew's coefficient is about 3.1, 3.2, 3.3, 3.4, 3.5, 3.6, 3.7, 3.8, or 3.9 Å³ per Da to about 4 Å³ per Da. Preferably, Matthew's coefficient is about 3.58 Å³ per Da. Solvent content is from about 40% to about 90%, preferably from about 55% to about 75%, preferably about 65%.

As used herein, the term “Neutrokine-alpha protein” includes naturally and recombinantly produced Neutrokine-alpha proteins; natural, synthetic, and recombinant biologically active polypeptide fragments of Neutrokine-alpha protein; biologically active polypeptide variants of Neutrokine-alpha protein or fragments thereof, including hybrid fusion proteins and dimers; biologically active polypeptide analogs of Neutrokine-alpha protein or fragments or variants thereof, including cysteine-substituted analogs. The Neutrokine-alpha protein may be generated and/or isolated by any means known in the art. Neutrokine-alpha proteins and methods of producing Neutrokine-alpha proteins are disclosed in U.S. Pat. Appl. Nos. 60/225,628, filed Aug. 15, 2000; 60/227,008, filed Aug. 23, 2000; 60/234,338, filed Sep. 22, 2000; 60/240,806, filed Oct. 17, 2000; 60/250,020, filed Nov. 30, 2000; 60/276,248, filed Mar. 6, 2001; 60/293,499, filed May 25, 2001; 60/296,122, filed Jun. 7, 2001; and 60/304,809, filed Jul. 13, 2001; all of which are fully incorporated by reference herein.

Preferably, the Neutrokine-alpha protein is a protein comprising, or alternatively consisting of, the sequence listed in Table 5, or is a homologue of the protein comprising, or alternatively consisting of, the sequence listed in Table 5.

The term “hNeutrokine-alpha” refers to human Neutrokine-alpha and preferentially refers to a protein comprising, or alternatively consisting of, the sequence listed in Table 5.

A homologue is a protein that may include one or more amino acid substitutions, deletions, or additions, either from natural mutations of human manipulation. Thus, a Neutrokine-alpha protein in crystalline form may include one or more amino acid substitutions, deletions or additions, either from natural mutations or human manipulation. As indicated, changes are preferably of a minor nature, such as conservative amino acid substitutions that do not significantly affect the folding or activity of the protein (see Table 1).

TABLE 1
Conservative Amino Acid Substitutions.

| Amino Acid Type | Examples of Amino Acids |
|---|---|
| Aromatic | Phenylalanine, Tryptophan, |
| | Tyrosine, Histidine |
| Hydrophobic | Leucine, Isoleucine, Valine, |
| | Methionine, Histidine |
| Polar | Glutamine, Asparagine, Serine, |
| | Cysteine, Threonine |
| Basic | Arginine, Lysine, Histidine |
| Acidic | Aspartic Acid, Glutamic Acid |
| Small | Alanine, Serine, Threonine, |
| | Methionine, Glycine |

In one embodiment of the invention, a Neutrokine-alpha protein in crystalline form comprises, or alternatively consists of, the amino acid sequence of a Neutrokine-alpha having an amino acid sequence which contains at least one conservative amino acid substitution, but not more than 0.50 conservative amino acid substitutions, even more preferably, not more than 40 conservative amino acid substitutions, still more preferably, not more than 30 conservative amino acid substitutions, and still even more preferably, not more than 20 conservative amino acid substitutions. Of course, in order of ever-increasing preference, it is highly preferable for the Neutrokine-alpha protein to have an amino acid sequence which comprises the amino acid sequence of human Neutrokine-alpha, which contains at least one, but not more than 10, 9, 8, 7, 6, 5, 4, 3, 2 or 1 conservative amino acid substitutions.

For example, site directed changes at the amino acid level of a Neutrokine-alpha protein can be made by replacing a particular amino acid with a conservative substitution. Preferred conservative substitution mutations of the Neutrokine-alpha amino acid sequence provided in Table 5 include: T141 replaced with A, G, I, L, S, M, or V; V142 replaced with A, G, I, L, S, T, or M; T143 replaced with A, G, I, L, S, M, or V; Q144 replaced with N; D145 replaced with E; L147 replaced with A, G, I, S, T, M, or V; Q148 replaced with N; L149 replaced with A, G, I, S, T, M, or V; 1150 replaced with A, G, L, S, T, M, or V; A151 replaced with G, I, L, S, T, M, or V; D152 replaced with E; S153 replaced with A, G, I, L, T, M, or V; E154 replaced with D; T155 replaced with A, G, I, L, S, M, or V; T157 replaced with A, G, I, L, S, M, or V; I158 replaced with A, G, L, S, T, M, or V; Q159 replaced with N; K160 replaced with H, or R; G161 replaced with A, I, L, S, T, M, or V; S162 replaced with A, G, I, L, T, M, or V; Y163 replaced with F, or W; T164 replaced with A, G, I, L, S, M, or V; F165 replaced with W, or Y; V166 replaced with A, G, I, L, S, T, or M; W168 replaced with F, or Y; L169 replaced with A, G, I, S, T, M, or V; L170 replaced with A, G, I, S, T, M, or V; S171 replaced with A, G, I, L, T, M, or V; F172 replaced with W, or Y; K173 replaced with H, or R; R174 replaced with H, or K; G175 replaced with A, I, L, S, T, M, or V; S176 replaced with A, G, I, L, T, M, or V; A177 replaced with G, I, L, S, T, M, or V; L178 replaced with A, G, I, S, T, M, or V; E179 replaced with D; E180 replaced with D; K181 replaced with H, or R; E182 replaced with D; N183 replaced with Q; K184 replaced with H, or R; I185 replaced with A, G, L, S, T, M, or V; L186 replaced with A, G, I, S, T, M, or V; V187 replaced with A, G, I, L, S, T, or M; K188 replaced with H, or R; E189 replaced with D; T190 replaced with A, G, I, L, S, M, or V; G191 replaced with A, I, L, S, T, M, or V; Y192 replaced with F, or W; F193 replaced with W, or Y; F194 replaced with W, or Y; 1195 replaced with A, G, L, S, T, M, or V; Y196 replaced with F, or W; G197 replaced with A, I, L, S, T, M, or V; Q198 replaced with N; V199 replaced with A, G, I, L, S, T, or M; L200 replaced with A, G, I, S, T, M, or V; Y201 replaced with F, or W; T202 replaced with A, G, I, L, S, M, or V; D203 replaced with E; K204 replaced with H, or R; T205 replaced with A, G, I, L, S, M, or V; Y206 replaced with F, or W; A207 replaced with G, I, L, S, T, M, or V; M208 replaced with A, G, I, L, S, T, or V; G209 replaced with A, I, L, S, T, M, or V; H210 replaced with K, or R; L211 replaced with A, G, I, S, T, M, or V; I212 replaced with A, G, L, S, T, M, or V; Q213 replaced with N; R214 replaced with H, or K; K215 replaced with H, or R; K216 replaced with H, or R; V217 replaced with A, G, I, L, S, T, or M; H218 replaced with K, or R; V219 replaced with A, G, I, L, S, T, or M; F220 replaced with W, or Y; G221 replaced with A, I, L, S, T, M, or V; D222 replaced with E; E223 replaced with D; L224 replaced with A, G, I, S, T, M, or V; S225 replaced with A, G, I, L, T, M, or V; L226 replaced with A, G, I, S, T, M, or V; V227 replaced with A, G, I, L, S, T, or M; T228 replaced with A, G, I, L, S, M, or V; L229 replaced with A, G, I, S, T, M, or V; F230 replaced with W, or Y; R231 replaced with H, or K; I233 replaced with A, G, L, S, T, M, or V; Q234 replaced with N; N235 replaced with Q; M236 replaced with A, G, I, L, S, T, or V; E238 replaced with D; T239 replaced with A, G, I, L, S, M, or V; L240 replaced with A, G, I, S, T, M, or V; N242 replaced with Q; N243 replaced with Q; S244 replaced with A, G, I, L, T, M, or V; Y246 replaced with F, or W; S247 replaced with A, G, I, L, T, M, or V; A248 replaced with G, I, L, S, T, M, or V; G249 replaced with A, I, L, S, T, M, or V; I250 replaced with A, G, L, S, T, M, or V; A251 replaced with G, I, L, S, T, M, or V; K252 replaced with H, or R; L253 replaced with A, G, I, S, T, M, or V; E254 replaced with D; E255 replaced with D; G256 replaced with A, I, L, S, T, M, or V; D257 replaced with E; E258 replaced with D; L259 replaced with A, G, I, S, T, M, or V; Q260 replaced with N; L261 replaced with A, G, I, S, T, M, or V; A262 replaced with G, I, L, S, T, M, or V; 1263 replaced with A, G, L, S, T, M, or V; R265 replaced with H, or K; E266 replaced with D; N267 replaced with Q; A268 replaced with G, I, L, S, T, M, or V; Q269 replaced with N; 1270 replaced with A, G, L, S, T, M, or V; S271 replaced with A, G, I, L, T, M, or V; L272 replaced with A, G, I, S, T, M, or V; D273 replaced with E; G274 replaced with A, I, L, S, T, M, or V; D275 replaced with E; V276 replaced with A, G, I, L, S, T, or M; T277 replaced with A, G, I, L, S, M, or V; F278 replaced with W, or Y; F279 replaced with W, or Y; G280 replaced with A, I, L, S, T, M, or V; A281 replaced with G, I, L, S, T, M, or V; L282 replaced with A, G, I, S, T, M, or V; K283 replaced with H, or R; L284 replaced with A, G, I, S, T, M, or V; and/or L285 replaced with A, G, I, S, T, M, or V. The resulting Neutrokine-alpha proteins may be routinely screened for Neutrokine-alpha functional activity and/or physical properties (such as, for example, enhanced or reduced stability and/or solubility). The resulting Neutrokine-alpha proteins may be used according the present invention as described herein.

In another embodiment, the invention provides for a Neutrokine-alpha protein in crystalline form having amino acid sequences containing non-conservative substitutions of the amino acid sequence provided in Table 5. For example, non-conservative substitutions of the Neutrokine-alpha protein sequence provided in Table 5 include: T141 replaced with D, E, H, K, R, N, Q, F, W, Y, P, or C; V142 replaced with D, E, H, K, R, N, Q, F, W, Y, P, or C; T143 replaced with D, E, H, K, R, N, Q, F, W, Y, P, or C; Q144 replaced with D, E, H, K, R, A, G, I, L, S, T, M, V, F, W, Y, P, or C; D145 replaced with H, K, R, A, G, I, L, S, T, M, V, N, Q, F, W, Y, P, or C; C146 replaced with D, E, H, K, R, A, G, I, L, S, T, M, V, N, Q, F, W, Y, or P; L147 replaced with D, E, H, K, R, N, Q, F, W, Y, P, or C; Q148 replaced with D, E, H, K, R, A, G, I, L, S, T, M, V, F, W, Y, P, or C; L149 replaced with D, E, H, K, R, N, Q, F, W, Y, P, or C; I150 replaced with D, E, H, K, R, N, Q, F, W, Y, P, or C; A151 replaced with D, E, H, K, R, N, Q, F, W, Y, P, or C; D152 replaced with H, K, R, A, G, I, L, S, T, M, V, N, Q, F, W, Y, P, or C; S153 replaced with D, E, H, K, R, N, Q, F, W, Y, P, or C; E154 replaced with H, K, R, A, G, I, L, S, T, M, V, N, Q, F, W, Y, P, or C; T155 replaced with D, E, H, K, R, N, Q, F, W, Y, P, or C; P156 replaced with D, E, H, K, R, A, G, I, L, S, T, M, V, N, Q, F, W, Y, or C; T157 replaced with D, E, H, K, R, N, Q, F, W, Y, P, or C; I158 replaced with D, E, H, K, R, N, Q, F, W, Y, P, or C; Q159 replaced with D, E, H, K, R, A, G, I, L, S, T, M, V, F, W, Y, P, or C; K160 replaced with D, E, A, G, I, L, S, T, M, V, N, Q, F, W, Y, P, or C; G161 replaced with D, E, H, K, R, N, Q, F, W, Y, P, or C; S162 replaced with D, E, H, K, R, N, Q, F, W, Y, P, or C; Y163 replaced with D, E, H, K, R, N, Q, A, G, I, L, S, T, M, V, P, or C; T164 replaced with D, E, H, K, R, N, Q, F, W, Y, P, or C; F165 replaced with D, E, H, K, R, N, Q, A, G, I, L, S, T, M, V, P, or C; V166 replaced with D, E, H, K, R, N, Q, F, W, Y, P, or C; P167 replaced with D, E, H, K, R, A, G, I, L, S, T, M, V, N, Q, F, W, Y, or C; W168 replaced with D, E, H, K, R, N, Q, A, G, I, L, S, T, M, V, P, or C; L169 replaced with D, E, H, K, R, N, Q, F, W, Y, P, or C; L170 replaced with D, E, H, K, R, N, Q, F, W, Y, P, or C; S171 replaced with D, E, H, K, R, N, Q, F, W, Y, P, or C; F172 replaced with D, E, H, K, R, N, Q, A, G, I, L, S, T, M, V, P, or C; K173 replaced with D, E, A, G, I, L, S, T, M, V, N, Q, F, W, Y, P, or C; R174 replaced with D, E, A, G, I, L, S, T, M, V, N, Q, F, W, Y, P, or C; G175 replaced with D, E, H, K, R, N, Q, F, W, Y, P, or C; S176 replaced with D, E, H, K, R, N, Q, F, W, Y, P, or C; A177 replaced with D, E, H, K, R, N, Q, F, W, Y, P, or C; L178 replaced with D, E, H, K, R, N, Q, F, W, Y, P, or C; E179 replaced with H, K, R, A, G, I, L, S, T, M, V, N, Q, F, W, Y, P, or C; E180 replaced with H, K, R, A, G, I, L, S, T, M, V, N, Q, F, W, Y, P, or C; K181 replaced with D, E, A, G, I, L, S, T, M, V, N, Q, F, W, Y, P, or C; E182 replaced with H, K, R, A, G, I, L, S, T, M, V, N, Q, F, W, Y, P, or C; N183 replaced with D, E, H, K, R, A, G, I, L, S, T, M, V, F, W, Y, P, or C; K184 replaced with D, E, A, G, I, L, S, T, M, V, N, Q, F, W, Y, P, or C; 1185 replaced with D, E, H, K, R, N, Q, F, W, Y, P, or C; L186 replaced with D, E, H, K, R, N, Q, F, W, Y, P, or C; V187 replaced with D, E, H, K, R, N, Q, F, W, Y, P, or C; K188 replaced with D, E, A, G, I, L, S, T, M, V, N, Q, F, W, Y, P, or C; E189 replaced with H, K, R, A, G, I, L, S, T, M, V, N, Q, F, W, Y, P, or C; T190 replaced with D, E, H, K, R, N, Q, F, W, Y, P, or C; G191 replaced with D, E, H, K, R, N, Q, F, W, Y, P, or C; Y192 replaced with D, E, H, K, R, N, Q, A, G, I, L, S, T, M, V, P, or C; F193 replaced with D, E, H, K, R, N, Q, A, G, I, L, S, T, M, V, P, or C; F194 replaced with D, E, H, K, R, N, Q, A, G, I, L, S, T, M, V, P, or C; I195 replaced with D, E, H, K, R, N, Q, F, W, Y, P, or C; Y196 replaced with D, E, H, K, R, N, Q, A, G, I, L, S, T, M, V, P, or C; G197 replaced with D, E, H, K, R, N, Q, F, W, Y, P, or C; Q198 replaced with D, E, H, K, R, A, G, I, L, S, T, M, V, F, W, Y, P, or C; V199 replaced with D, E, H, K, R, N, Q, F, W, Y, P, or C; L200 replaced with D, E, H, K, R, N, Q, F, W, Y, P, or C; Y201 replaced with D, E, H, K, R, N, Q, A, G, I, L, S, T, M, V, P, or C; T202 replaced with D, E, H, K, R, N, Q, F, W, Y, P, or C; D203 replaced with H, K, R, A, G, I, L, S, T, M, V, N, Q, F, W, Y, P, or C; K204 replaced with D, E, A, G, I, L, S, T, M, V, N, Q, F, W, Y, P, or C; T205 replaced with D, E, H, K, R, N, Q, F, W, Y, P, or C; Y206 replaced with D, E, H, K, R, N, Q, A, G, I, L, S, T, M, V, P, or C; A207 replaced with D, E, H, K, R, N, Q, F, W, Y, P, or C; M208 replaced with D, E, H, K, R, N, Q, F, W, Y, P, or C; G209 replaced with D, E, H, K, R, N, Q, F, W, Y, P, or C; H210 replaced with D, E, A, G, I, L, S, T, M, V, N, Q, F, W, Y, P, or C; L211 replaced with D, E, H, K, R, N, Q, F, W, Y, P, or C; I212 replaced with D, E, H, K, R, N, Q, F, W, Y, P, or C; Q213 replaced with D, E, H, K, R, A, G, I, L, S, T, M, V, F, W, Y, P, or C; R214 replaced with D, E, A, G, I, L, S, T, M, V, N, Q, F, W, Y, P, or C; K215 replaced with D, E, A, G, I, L, S, T, M, V, N, Q, F, W, Y, P, or C; K216 replaced with D, E, A, G, I, L, S, T, M, V, N, Q, F, W, Y, P, or C; V217 replaced with D, E, H, K, R, N, Q, F, W, Y, P, or C; H218 replaced with D, E, A, G, I, L, S, T, M, V, N, Q, F, W, Y, P, or C; V219 replaced with D, E, H, K, R, N, Q, F, W, Y, P, or C; F220 replaced with D, E, H, K, R, N, Q, A, G, I, L, S, T, M, V, P, or C; G221 replaced with D, E, H, K, R, N, Q, F, W, Y, P, or C; D222 replaced with H, K, R, A, G, I, L, S, T, M, V, N, Q, F, W, Y, P, or C; E223 replaced with H, K, R, A, G, I, L, S, T, M, V, N, Q, F, W, Y, P, or C; L224 replaced with D, E, H, K, R, N, Q, F, W, Y, P, or C; S225 replaced with D, E, H, K, R, N, Q, F, W, Y, P, or C; L226 replaced with D, E, H, K, R, N, Q, F, W, Y, P, or C; V227 replaced with D, E, H, K, R, N, Q, F, W, Y, P, or C; T228 replaced with D, E, H, K, R, N, Q, F, W, Y, P, or C; L229 replaced with D, E, H, K, R, N, Q, F, W, Y, P, or C; F230 replaced with D, E, H, K, R, N, Q, A, G, I, L, S, T, M, V, P, or C; R231 replaced with D, E, A, G, I, L, S, T, M, V, N, Q, F, W, Y, P, or C; C232 replaced with D, E, H, K, R, A, G, I, L, S, T, M, V, N, Q, F, W, Y, or P; I233 replaced with D, E, H, K, R, N, Q, F, W, Y, P, or C; Q234 replaced with D, E, H, K, R, A, G, I, L, S, T, M, V, F, W, Y, P, or C; N235 replaced with D, E, H, K, R, A, G, I, L, S, T, M, V, F, W, Y, P, or C; M236 replaced with D, E, H, K, R, N, Q, F, W, Y, P, or C; P237 replaced with D, E, H, K, R, A, G, I, L, S, T, M, V, N, Q, F, W, Y, or C; E238 replaced with H, K, R, A, G, I, L, S, T, M, V, N, Q, F, W, Y, P, or C; T239 replaced with D, E, H, K, R, N, Q, F, W, Y, P, or C; L240 replaced with D, E, H, K, R, N, Q, F, W, Y, P, or C; P241 replaced with D, E, H, K, R, A, G, I, L, S, T, M, V, N, Q, F, W, Y, or C; N242 replaced with D, E, H, K, R, A, G, I, L, S, T, M, V, F, W, Y, P, or C; N243 replaced with D, E, H, K, R, A, G, I, L, S, T, M, V, F, W, Y, P, or C; S244 replaced with D, E, H, K, R, N, Q, F, W, Y, P, or C; C245 replaced with D, E, H, K, R, A, G, I, L, S, T, M, V, N, Q, F, W, Y, or P; Y246 replaced with D, E, H, K, R, N, Q, A, G, I, L, S, T, M, V, P, or C; S247 replaced with D, E, H, K, R, N, Q, F, W, Y, P, or C; A248 replaced with D, E, H, K, R, N, Q, F, W, Y, P, or C; G249 replaced with D, E, H, K, R, N, Q, F, W, Y, P, or C; I250 replaced with D, E, H, K, R, N, Q, F, W, Y, P, or C; A251 replaced with D, E, H, K, R, N, Q, F, W, Y, P, or C; K252 replaced with D, E, A, G, I, L, S, T, M, V, N, Q, F, W, Y, P, or C; L253 replaced with D, E, H, K, R, N, Q, F, W, Y, P, or C; E254 replaced with H, K, R, A, G, I, L, S, T, M, V, N, Q, F, W, Y, P, or C; E255 replaced with H, K, R, A, G, I, L, S, T, M, V, N, Q, F, W, Y, P, or C; G256 replaced with D, E, H, K, R, N, Q, F, W, Y, P, or C; D257 replaced with H, K, R, A, G, I, L, S, T, M, V, N, Q, F, W, Y, P, or C; E258 replaced with H, K, R, A, G, I, L, S, T, M, V, N, Q, F, W, Y, P, or C; L259 replaced with D, E, H, K, R, N, Q, F, W, Y, P, or C; Q260 replaced with D, E, H, K, R, A, G, I, L, S, T, M, V, F, W, Y, P, or C; L261 replaced with D, E, H, K, R, N, Q, F, W, Y, P, or C; A262 replaced with D, E, H, K, R, N, Q, F, W, Y, P, or C; I263 replaced with D, E, H, K, R, N, Q, F, W, Y, P, or C; P264 replaced with D, E, H, K, R, A, G, I, L, S, T, M, V, N, Q, F, W, Y, or C; R265 replaced with D, E, A, G, I, L, S, T, M, V, N, Q, F, W, Y, P, or C; E266 replaced with H, K, R, A, G, I, L, S, T, M, V, N, Q, F, W, Y, P, or C; N267 replaced with D, E, H, K, R, A, G, I, L, S, T, M, V, F, W, Y, P, or C; A268 replaced with D, E, H, K, R, N, Q, F, W, Y, P, or C; Q269 replaced with D, E, H, K, R, A, G, I, L, S, T, M, V, F, W, Y, P, or C; I270 replaced with D, E, H, K, R, N, Q, F, W, Y, P, or C; S271 replaced with D, E, H, K, R, N, Q, F, W, Y, P, or C; L272 replaced with D, E, H, K, R, N, Q, F, W, Y, P, or C; D273 replaced with H, K, R, A, G, I, L, S, T, M, V, N, Q, F, W, Y, P, or C; G274 replaced with D, E, H, K, R, N, Q, F, W, Y, P, or C; D275 replaced with H, K, R, A, G, I, L, S, T, M, V, N, Q, F, W, Y, P, or C; V276 replaced with D, E, H, K, R, N, Q, F, W, Y, P, or C; T277 replaced with D, E, H, K, R, N, Q, F, W, Y, P, or C; F278 replaced with D, E, H, K, R, N, Q, A, G, I, L, S, T, M, V, P, or C; F279 replaced with D, E, H, K, R, N, Q, A, G, I, L, S, T, M, V, P, or C; G280 replaced with D, E, H, K, R, N, Q, F, W, Y, P, or C; A281 replaced with D, E, H, K, R, N, Q, F, W, Y, P, or C; L282 replaced with D, E, H, K, R, N, Q, F, W, Y, P, or C; K283 replaced with D, E, A, G, I, L, S, T, M, V, N, Q, F, W, Y, P, or C; L284 replaced with D, E, H, K, R, N, Q, F, W, Y, P, or C; and/or L285 replaced with D, E, H, K, R, N, Q, F, W, Y, P, or C. The resulting Neutrokine-alpha protein in crystalline form may be routinely screened for Neutrokine-alpha functional activities and/or physical properties (such as, for example, enhanced or reduced stability and/or solubility and/or oligomeric state) described throughout the specification and known in the art. Preferably, the resulting proteins of the invention have an increased and/or a decreased Neutrokine-alpha functional activity. More preferably, the resulting Neutrokine-alpha proteins of the invention have more than one increased and/or decreased Neutrokine-alpha functional activity and/or physical property.

In an additional embodiment, a Neutrokine-alpha protein in crystalline form of the present invention comprises, or alternatively consists of, a Neutrokine-alpha protein with more than one amino acid (e.g., 2, 3, 4, 5, 6, 7, 8, 9, 10, 15, 20, 30 and 50) replaced with the substituted amino acids as described above (either conservative or nonconservative).

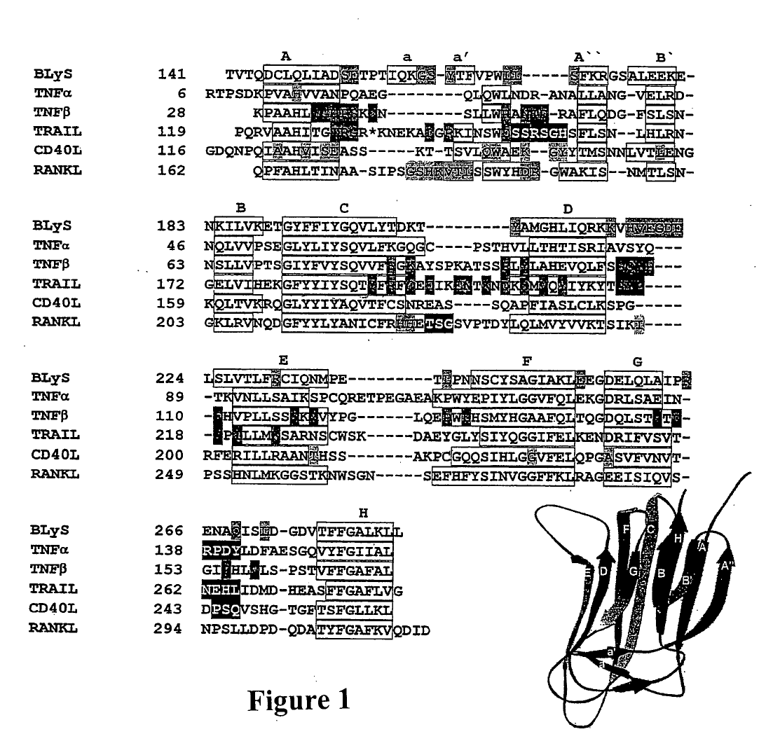

Preferred modified Neutrokine-alpha proteins include a protein having the sequence as listed in FIG. 1A with one or more of the following amino acid residues mutated: V-142; T-143; Q-144; D-145; C-146; L-147; Q-148; L-149; I-150; A-151; D-152; S-153; E-154; T-155; P-156; T-157; I-158; Q-159; and K-160.

By a protein having an amino acid sequence at least, for example, 95% “identical” to a reference amino acid sequence of a Neutrokine-alpha protein is intended that the amino acid sequence of the protein is identical to the reference sequence except that the protein sequence may include up to five amino acid alterations per each 100 amino acids of the reference amino acid of the Neutrokine-alpha protein. In other words, to obtain a protein having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide or protein is at least 80%, 85%, 90%, 95%, 96%, 97%, 98% or 99% identical to, for instance, the amino acid sequences shown in TABLES 4 and 5, or fragments thereof, can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. Comp. App. Biosci. 6:237-245 (1990). Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made for the purposes of this embodiment.

An additional aspect of the present invention is a composition comprising a Neutrokine-alpha protein that is suitable for producing a Neutrokine-alpha protein in crystalline form.

Protein Crystallization Methods

The present invention provides methods for preparing a Neutrokine-alpha protein in crystalline form. Preferably, the method produces a Neutrokine-alpha protein in crystalline form, wherein said Neutrokine-alpha protein diffracts X-rays with sufficiently high resolution to allow determination of the three-dimensional structure of said Neutrokine-alpha protein product, including atomic coordinates. The three-dimensional structure is useful in a number of methods of the present invention, as described herein. Specifically provided is a method for crystallizing a recombinant, non-glycosylated human Neutrokine-alpha protein comprising the amino acid sequence listed in FIG. 1A and Table 5.

Said protein can be obtained from suitable sources, such as eukaryotic cells or tissues. In general, a protein comprising a Neutrokine-alpha protein or a portion thereof is isolated in soluble form in sufficient purity and concentrated for crystallization. The polypeptide is optionally assayed for lack of aggregation (which may interfere with crystallization). The purified polypeptide is preferably crystallized under varying conditions of at least one of the following factors: pH, buffering agent, buffer concentration, salt, polymer, polymer concentration, other precipitating agents, and concentration of purified Neutrokine-alpha protein or portion thereof. See, e.g., Blundell et al., Protein Crystallography, Academic Press, London (1976); McPherson, The Preparation and Analysis of Protein Crystals, Wiley Interscience, N.Y. (1982). The crystallized polypeptide is optionally tested for Neutrokine-alpha activity and differently sized and shaped crystals are further tested for suitability for X-ray diffraction. Generally, larger crystals provide better crystallographic data than smaller crystals, and thicker crystals provide better crystallographic data than thinner crystals.

The pH of the solution is from about 4-9, preferably from about 6-7. Preferably, the pH of the solution is about 6.

The buffering agent can be any buffering agent. Buffering agents are well-known in the art. Exemplary buffering agents include citrate, phosphate, cacodylate, acetates, imidazole, Tris HCl, and sodium HEPES.

The buffer concentration is from about 10 millimolar (mM) to about 200 mM. Alternatively, the buffer concentration is about 10, 20, 30, 40, 50, 60, 70, 80, 90, 100, 110, 120, 130, 140, 150, 160, 170, 180, 190 or 200 mM.

The salt is an ionic salt, which is well known in the art. Exemplary salts include calcium chloride, sodium citrate, magnesium chloride, ammonium acetate, ammonium sulfate, potassium phosphate, magnesium acetate, zinc acetate, and calcium acetate.

The polymer is a compound that contains repeating subunits. Exemplary polymers that are useful in the present invention include polyethylene glycol (PEG), polypropyleneglycol (PPG), and others. The average molecular weight of the polymer is from about 200 to about 100,000. Other suitable values for the average molecular weight of the polymer include from about 200 to about 10,000; from about 1,000 to about 10,000; from about 5,000 to about 100,000; from about 5,000 to about 10,000.

The concentration of the polymer is the concentration of the polymer in the solution suitable for crystallization. The concentration of the polymer is from about 1% to about 50%. The concentration of the polymer is about 1%, 5%, 10%, 15%, 20%, 25%, 30%, 35%, 40%, 45%, or 50%.

The solution suitable for crystallization optionally comprises one or more additional agents selected from the group consisting of potassium tartrate, sodium tartrate, ammonium sulfate (NH₄SO₄), sodium acetate (CH₃CO₂Na), lithium sulfate (LiSO₄), sodium formate (HCO₂Na), sodium citrate, magnesium formate ((HCO₂)₂Mg), sodium phosphate, potassium phosphate; NH₄PO₄; 2-propanol; 2-methyl-2,4-pentanediol; and dioxane.

According to the present invention, the solution preferably contains dioxane. The concentration of the dioxane is from about 10% to about 60%, preferably from about 20% to about 50%, preferably from 30% to about 40%, preferably about 35%.

Any suitable crystallization method is used for crystallizing the Neutrokine-alpha protein or portion thereof, such as the hanging-drop, vapor diffusion method, microbatch, sitting drop, and dialysis. Preferably, hanging drop method is used. The crystals should be grown for from about 6 hours to about 72 hours.

According to the present invention, a preferred method of preparing a Neutrokine-alpha protein in crystalline form uses hanging drops containing about 1 mL of about 20 mg/mL hNeutrokine-alpha in about 25 mM sodium citrate, about 125 mM NaCl, pH of about 6 and about 1 ml of about 25% dioxane, about 25 mM MgCl₂ suspended over a reservoir of about 25% dioxane and about 25 mM MgCl₂.

According to the present invention, a preferred method of preparing a Neutrokine-alpha protein in crystalline form uses hanging drops containing about 1 μL of about 20 mg/mL hNeutrokine-alpha in about 25 mM sodium citrate, about 125 mM NaCl, pH of about 6 and about 1 μl of about 25% dioxane, about 25 mM MgCl₂ suspended over a reservoir of about 25% dioxane and about 25 mM MgCl₂.

Crystals grown according to the present invention diffract X-rays to at least 10 Å resolution, such as 0.15-10.0 Å, or any range of value therein, such as 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4 or 3.5, with 3.5 Å or higher resolution being preferred for determining the crystal structure. However, diffraction patterns with a lower resolution, such as 25-3.5 Å, are also useful.

According to the present invention, during growth, some of the crystals are optionally removed, washed, and assayed for biological activity. Other washed crystals are optionally run on a gel and stained, and those that migrate at the same molecular weight as the corresponding purified polypeptide comprising the Neutrokine-alpha protein or portion thereof are preferably used. From one to two hundred crystals can be observed in one drop. When fewer crystals are produced in a drop, the crystals may be a much larger size, for example from about 0.1 to about 0.4 mm

Heavy atom derivatives used for multiple isomorphous replacement are obtained by either soaking the crystals with a mercurial reagent or placing crystals in a gaseous xenon (Xe) atmosphere during data collection (Schiltz et al., J. Appl. Cryst. 27: 950-960 (1994)). Suitable mercurial reagents include sodium p-chloromercuribenzylsulphonate (PCMBS). The concentration of the mercurial reagent is from about 0.1 mM to about 0.5 mM or from about 0.1 mM to about 10 mM.

X-Ray Crystallography

Another aspect of the present invention is directed to determining the three-dimensional structure of a Neutrokine-alpha protein by using X-ray diffraction crystallography methods. The X-ray diffraction patterns can be either analyzed directly to provide the three-dimensional structure (if sufficient data are collected), or atomic coordinates for human Neutrokine-alpha protein in crystalline form, as provided herein, can be used for structure determination. The X-ray diffraction patterns obtained by methods of the present invention, and optionally provided on computer readable media, are used to provide electron density maps. The amino acid sequence is also useful for three-dimensional structure determination. The data are then used in combination with phase determination (e.g., using multiple isomorphous replacement (MIR) molecular replacement techniques) to generate electron density maps of Neutrokine-alpha, using a suitable computer system.

The electron density maps, provided by analysis of either the X-ray diffraction patterns or working backwards from the atomic coordinates, provided herein, are then fitted using suitable computer algorithms to generate secondary, tertiary, and/or quaternary structures and/or domains of Neutrokine-alpha, which structures and/or domains are then used to provide an overall three-dimensional structure, as well as binding sites of Neutrokine-alpha.

A Neutrokine-alpha protein in crystalline form produced according to the present invention is X-ray analyzed using a suitable X-ray source to obtain diffraction patterns. Preferably, said crystalline Neutrokine-alpha protein is used which is stable for at least 10 hrs in the X-ray beam. Frozen crystalline Neutrokine-alpha (e.g., −220 to −50° C.) is optionally used for longer X-ray exposures (e.g., 5-72 hrs), the crystals being relatively more stable to the X-rays in the frozen state. To collect the maximum number of useful reflections, preferably multiple frames are collected as the crystal is rotated in the X-ray beam. Larger crystals of crystalline Neutrokine-alpha (i.e., greater than about 150 μm) are preferred to increase the resolution of the X-ray diffraction patterns obtained. In one embodiment, crystals are analyzed using a synchrotron high energy X-ray source. Using frozen crystals, X-ray diffraction data are collected on crystals that diffract to at least a relatively high resolution of about 10 Å to about 1.5 Å. Diffraction data may also be collected on crystals that diffract at lower resolutions, such as from about 25 to about 10 Å.

Passing an X-ray beam through a crystal produces a diffraction pattern as a result of the X-rays interacting and being scattered by the contents of the crystal. The diffraction pattern are visualized using a method well-known in the art, e.g., an image plate or film, resulting in an image with spots corresponding to the diffracted X-rays. The positions of the spots in the diffraction pattern are used to determine parameters intrinsic to the crystal (such as unicell parameters) and to gain information on the packing of the molecules in the crystal. The intensity of the spots contains the Fourier transformation of the molecules in the crystal, i.e., information on the position of each atom in the crystal and hence of the crystallized molecule.

Although the diffraction patterns are usually themselves sufficient for three-dimensional structure determination, the amino acid sequence of the Neutrokine-alpha protein is also useful. The electron density maps, provided by analysis of the X-ray diffraction patterns, are then fitted using suitable computer algorithms as described below to generate secondary, tertiary and/or quaternary structure of the Neutrokine-alpha protein providing an overall three-dimensional model.

After data collection of diffraction patterns, the data are processed using methods well known in the art. One such suitable method to process the diffraction data is the MarXDS package Kabsch, W. J. Appl. Crystallogr. 21:916-924(1988)). The MarXDS package is a Fortran program developed for the reduction of single-crystal diffraction data from a sequence of adjacent rotation pictures recorded at a fixed X-ray wavelength by an electronic area detector. Patterson and cross Fourier analyses and SIR phasing can be performed using programs from the CCP4 package (Collaborative Computational Project No. 4, Acta Cryst. D50.760-763 (1994)), which is a suite of programs for the reduction and analysis of intensity data, structure solution by isomorphous replacement and molecular replacement, least-squares refinement, analysis of the structure, displaying electron-density maps and plotting molecules.

Electron density maps can be calculated using one of several well-known programs, such as those from the CCP4 computing package described above. Cycles of two-fold averaging can further be used, such as with the program RAVE (Kleywegt & Jones, Bailey et al., eds., First Map to Final Model, SERC Daresbury Laboratory, UK, pp. 59-66 (1994)) and gradual model expansion. The interpretation of electron density maps phased by multiple isomorphous replacement (MIR) to produce an initial molecular model is a critical step during the model building process. Three-dimensional computer graphics workstations are now widely used in the art for constructing models in MIR maps. One computer program in particular, FRODO, is commonly used and is available on a range of workstations (Jones, T. A. J. Appl. Cryst. 11:268-272 (1978)). In an attempt to improve the ability to interpret maps and then to construct more accurate models, Jones & Thirup, EMBO J. 5:819-822 (1986), introduced the use of skeletons coupled with a protein database of the best refined protein structures to build the initial model. This work suggested that all protein models could be built from fragments of existing structures. Jones et al. (Jones et al., Acta Cryst. A47:110-119 (1991)), extended these ideas with a computer graphics program called “O,” which allows the user to go from an initial C_{α} trace to a well refined model. An overview of the strategy used is provided below:

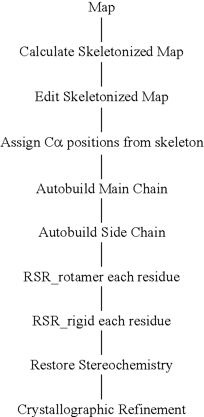

The three-dimensional structure of a Neutrokine-alpha protein can be built into a 3 Å resolution map through several cycles of model building using the “O” graphics program and phase combination using the Sigma A algorithm, which is part of the CCP4 package discussed above.

Refinement and Model Validation. Rigid body and positional refinement can be carried out using a program such as X-PLOR (Brünger, A. T., X-PLOR Version 3.1, Yale University Press (1992)) to a suitable crystallographic R_{factor}. If the model at this stage in the averaged maps still misses residues (e.g., at least 5-10 per subunit), then some or all of the missing residues can be incorporated in the model during additional cycles of positional refinement and model building. The refinement procedure can start using data from lower resolution (e.g., 25-10 Å to 10-3.0 Å) and then gradually be extended to include data from 12-6 Å to 3.0-1.5 Å. B-values (also termed temperature factors) for individual atoms can be refined once data of 2.8 Å or higher (e.g., up to 1.0 or 1.5 Å) has been added. Subsequently waters can be gradually added. A program such as ARP (Lamzin and Wilson, Acta Cryst. D49: 129-147 (1993)) can be used to add crystallographic waters and as a tool to check for bad areas in the model. Programs such as PROCHECK (Lackowski et al., J. Appl. Cryst. 26:283-291 (1993)), WHATIF (Vriend, J. Mol. Graph. 8:52-56 (1990)) and PROFILE 3D (Lüthy et al., Nature 356:83-85 (1992)), as well as the geometrical analysis generated by X-PLOR can be been used to check the structure for errors. A program such as DSSP can be used to assign the secondary structure elements (Kabsch and Sander, Biopolymers 22:2577-2637 (1983)). The model data are then saved on computer readable media for use in further analysis, such as, for example, in a method for modeling the structure of a related Neutrokine-alpha protein or in a computer-based system for the rational design of ligand that bind to, mimic, or inhibit a Neutrokine-alpha protein.

In general, X-ray diffraction data processing includes measuring the spots on each diffraction pattern in terms of position and intensity. This information is processed as indicated above (i.e., mathematical operations are performed on the data (such as scaling, merging and converting the data from intensity of diffracted beams to amplitudes)) to yield a set of data which is in a form as can be used for the further structure determination of the molecule. The amplitudes of the diffracted X-rays are then combined with calculated phases to produce an electron density map of the contents of the crystal. In the electron density map, the structure of the molecules (as present in the crystal) is built. The phases can be determined with various known techniques, one being molecular replacement.

For the molecular replacement technique, one takes a known three dimensional structure thought to share structural homology with the structure to be determined, to generate, after calculations, a first set of initial phases. These phases can be combined with the diffraction information of the molecule whose structure you want to solve.

The phases can be further optimized using a technique called density modification, which allows electron density maps of better quality to be produced facilitating interpretation and model building therein. The model is then refined by allowing the atoms in the model to move in order to match the diffraction data as well as possible while continuing to satisfy stereochemical constraints, such as reasonably bond lengths and bond angles.

In general, the R factor is preferably between about 0.15 and about 0.35 for a well-determined structure of a Neutrokine-alpha protein. The residual difference is a consequence of errors and imperfections in the data. These derive from various sources, including slight variations in the conformation of the protein molecules, as well as inaccurate corrections both for the presence of solvent and for differences in the orientation of the microcrystals from which the crystal is built.

Three-Dimensional Structure of Human Neutrokine-Alpha (hNeutrokine-Alpha)

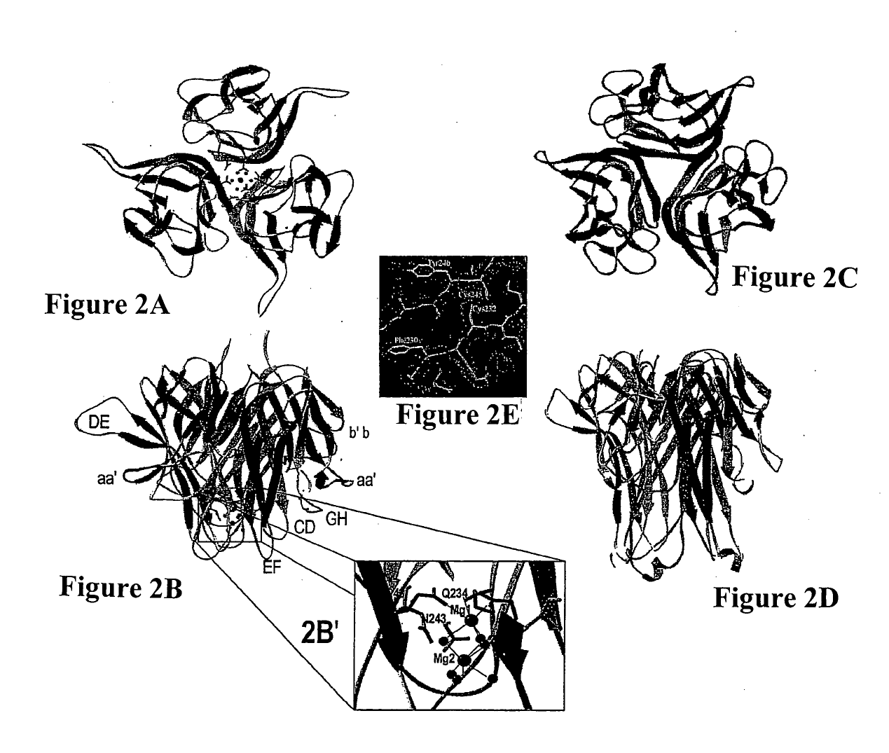

The monomer of hNeutrokine-alpha adopts the TNF-like jellyroll fold consisting of two five-stranded β-sheets with similar arrangement as the other representatives of this family. A structure-based sequence alignment among members of this cytokine family (see FIG. 1A) reveals that the Greek-key motif of the strands is conserved throughout the family despite the low identity in sequence. Using these structural alignments, the calculated identities between hNeutrokine-alpha and the other TNF-like proteins are: about 15% to TNF-α, about 16% to CD40L, about 19% TRANCE/RANKL, about 18% to Apo2L/TRAIL, and about 20% to TNF-β. The identities occur primarily in the β-strands C, D, F, G, and H that constitute the core of the jellyroll fold (see FIG. 1B). However, major differences are observed in the loop regions AA″, CD, DE, EF, and GH of the related cytokines. In contrast with related cytokines, hNeutrokine-alpha does not have the short GH α-helix, is truncated in loops CD and EF, and contains large inserts between strands A and A″ and between strands D and E. In hNeutrokine-alpha, the AA″ loop is modified by insertion of two short β-strands forming a hairpin motif (a and a′, FIG. 1B) that does not participate in β-sheet formation but widens the molecule. Similarly, the DE loop that has a four-residue insert, protrudes from the surface and forms inter-trimer contacts reminiscent of a handshake. As a result of these differences, the hNeutrokine-alpha homotrimer measures about 52 Å high (along the three-fold axis) and about 60 Å wide as compared to about 58 Å and about 57 Å, respectively, in TNF-β (see FIG. 2B). A sample of the experimental electron density is shown in FIG. 2E, in the region of the disulfide bond between residues 232 and 245. This disulfide bond holds strands E and F together, thereby stabilizing loop EF. The disulfide bond found in both TNF-α and CD40L connect loops CD and EF. Three hneutrokine-alpha monomers make extensive contacts within the trimer (about 5700 Å² of buried surface) with the sheets inclined about 30° relative to the three-fold axis (FIG. 2A). By analogy with other cytokine-receptor complexes, the narrow end of the trimer (displaying the CD and EF loops) is predicted to be proximal to the B-cell membrane when hNeutrokine-alpha is bound to its receptor(s).

A complex of two hydrated Mg²⁺ ions binds to the hNeutrokine-alpha trimer along the three-fold axis, near the trimer's narrow end (FIG. 2A). A complex formation of two magnesium ions bound to the protein is observed (FIG. 2B). One ion (Mg1) is bound to the side chains of Gln234 residues from each monomer and interacts with the other (Mg2) via bridging water molecules (FIG. 2B′). The water molecules are bound to the protein via residues N243 and the main chain oxygen of N235. A zinc ion was identified in a related position (about 6.4 Å from Mg1) in the Apo2L/TRAIL, along the three-fold axis, interacting with Cys230 sulfhydryls from each monomer (Hymowitz et al., Biochemistry 39:633-640 (2000). Mutating residue Q234 to X had deleterious effects on the formation of the hNeutrokine-alpha trimer, resulting in aggregation. The metal ions are assigned to be magnesium because a) the crystals were grown in a solution containing 25 mM MgCl₂, b) each metal ion coordinates 6 oxygen atoms, and c) the B refined factors are reasonable (about 28-33 Å²) for magnesium. Other molecules were also observed bound to the protein. Dioxane molecules were found along the three-fold axis interacting with phenyl rings of Phe165 and Phe194. Also, a citrate molecule was located at the interface between two trimers in the asymmetric unit where the DE loops shake hands and is situated on a local two-fold axis and is two-fold disordered. The carboxylates of the citrate bind to His218, Arg214, Glu223, LYS252, ASP254, and LYS216.

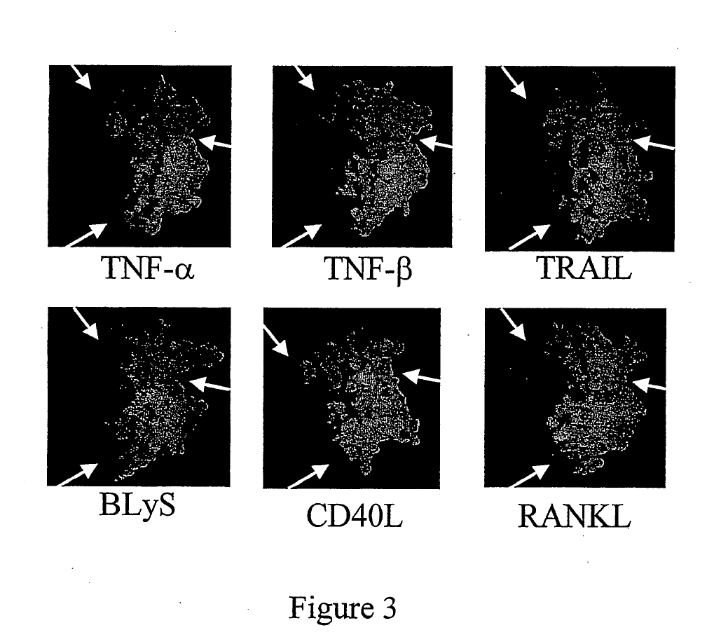

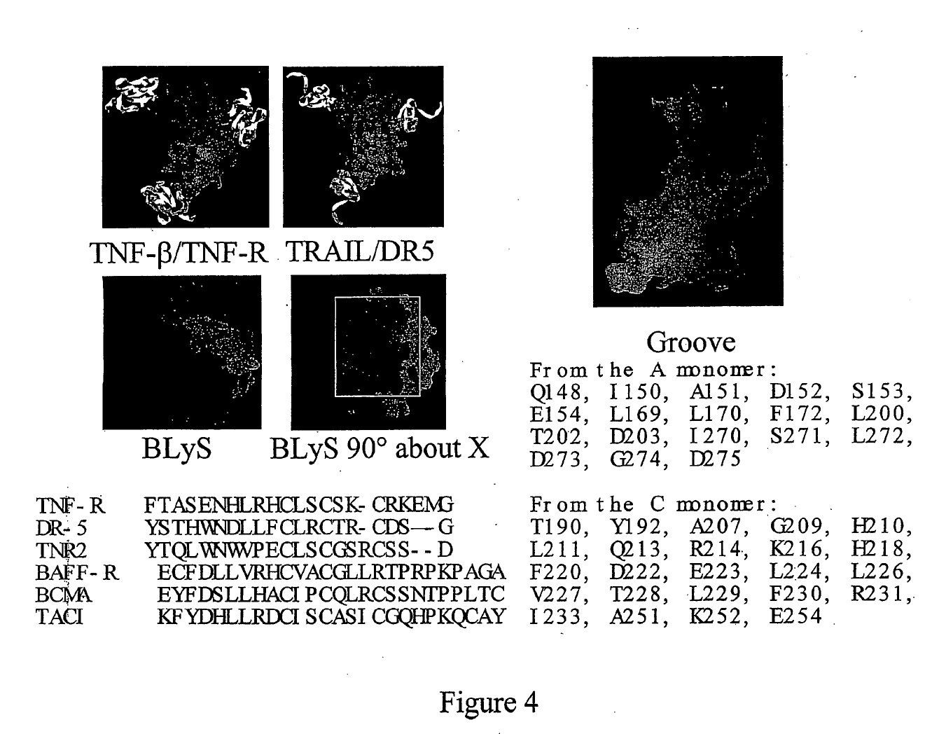

A comparison of the molecular surface of the biologically active trimeric form of hNeutrokine-alpha (FIG. 3) to that of other cytokines has revealed that this protein has a unique shape with three pronounced grooves on the surface. A similar shape is found in the other cytokines but in none is it as extensive or as deep. The groove winds around the surface of the trimer and has a shape appropriate for binding elongated receptors. As seen in FIG. 3, the TNF-R and the DR5 receptors bind to this region of the cytosine. This putative receptor-binding site is created by loops from two monomers coming together to each form the sides of the groove. The walls of this groove consist on one side of loop DE with some residues of loops aa′ and GH, and on the other side are found loops EF, Aa, and a′A″. These residues are highly variable within the TNF family. In the structures of cytokines complexed to their receptors (PDB entries 1TNR and 1D4V or 1D0G), these loops form the most extensive contacts within the complexes. The protruding DE loop that is unique to Neutrokine-alpha and the additional β-hairpin in the AA″ loop of Neutrokine-alpha when docked onto the TNF/TNF-R structure come in close contact along the ridges of the groove (FIGS. 4A and 4B). These residues would discriminate between TNF (or other cytokines) and Neutrokine-alpha, which does not bind to TNF-R.

The three receptors known to bind and be activated by Neutrokine-alpha share little sequence identity, yet they all contain at least one cysteine-rich domain. As seen in the complex between TNF and TNF-R, the receptor's cysteine-rich region (FIG. 4C) forms contacts with loops AA″ and DE of TNF. Baff-R, the receptor with the highest affinity towards Neutrokine-alpha, is the shortest sequence, containing only one cysteine-rich domain. An alignment of the cysteine-rich regions of BAFF-R, BCMA, and TACI that align best with the TNF-R recognition region is shown in FIG. 4C. The cysteines are structural and are somewhat conserved. The cysteine pair formed by the 3rd and 5th cysteines is found in all but BAFF-R. The Neutrokine-alpha receptors all contain proline residues that may shorten this β-strand (residues 60-80 of TNF-R). The recognition residues on TNF-R within this stretch are all unique to TNF-R which could explain the discriminatory ability of the receptors. The sequence in FIG. 4 is an elongated strand running from residue 65 to residue 80 and extends about 32.5 Å in length before turning at either end. Residues 55-59 and 69-81 contact the AA″ loop of TNF while residues 75-81 contact loops CD and GH. Loop DE binds to residues 60-70.

The structure of Neutrokine-alpha determined to 2 Å resolution reveals a distinctive binding groove at the interfaces between adjacent monomers in the trimer. This binding groove may allow the cytokine to discriminate between receptors. Receptors that cannot access the deep crevice may be excluded from binding. The receptor residues that participate in specific recognition of Neutrokine-alpha might be part of the consensus sequence: ExFDxLLRxCxxCxLxxT(S)xxPKP.

The groove is created by loops from two adjacent monomers. One wall of the groove contains loop DE with some residues of loops aa′ and GH, and the other wall of the groove contains loops EF, Aa, and a′A″. The deepest portion of the groove consists primarily of beta-strands D, E, and F. Residues with surface accessible side chains are ALA207, LEU211, GLN213, and ARG214 from strand D; THR228, LEU229, PHE230, ARG231, and ILE233 from strand E; and ALA251, LYS252, LEU253, GLU254, and ASP257 from strand F. The groove winds around the surface of the trimer and has a shape appropriate for binding elongated receptors. Loops DE and AA″ form the most extensive contacts with cytokine receptors. Modeling interactions of neutrokine-alpha with TNF-R indicate that the outer rim of the groove (loops DE and the beta-hairpin of loop AA″) would lead to steric conflict. These residues would permit receptors to discriminate between TNF or other cytokines and neutrokine-alpha. The residues involved in creating the surface of this groove and putative receptor-binding site are from adjacent monomers (green, FIG. 4a). Of those residues, the homology APRIL shares residues Leu 200, Arg 214, Thr 228, Leu 229, Phe 230, Arg 231, Ile 233, Leu 253, Asp 257 and Phe 278 with neutrokine-alpha (FIG. 4a, red). The majority of these shared residues are located on the floor of the groove, suggesting that the floor is used as a common binding motif for TACI, BCMA and BAFF-R to neutrokine-alpha and APRIL. Variations in residues on the groove walls would permit BAFF-R to discriminate against APRIL.

The three receptors known to bind and be activated by neutrokine-alpha share little sequence identity, but they all contain at least one Cys-rich domain. As seen in the complex between TNF and TNF-R, the Cys-rich region of the receptor forms contacts with loops AA″ and DE of TNF-alpha. BAFF-R, the receptor with the highest affinity for neutrokine-alpha, has the shortest sequence, containing only one Cys-rich domain. A ProDom24 database search (aided by PredictProtein25) probed using the BAFF-R sequence revealed BCMA as the most similar, specifically in the Cys-rich region, the transmembrane domain and an intracellular portion consisting of residues GEDPGTTPGHSVPVPA. In a receptor-binding study using SELDI affinity mass spectrometry26, we show that the a′A″ loop, the B′ and B strands, and strands C and D of the molecule are centrally involved (FIG. 4b) in the interaction of BlyS with both recombinant BCMA and TACI receptors, as indicated by the relatively large number of retained fragments of neutrokine-alpha that map to these areas. The data support the assumption that neutrokine-alpha interacts similarly with its receptors as other TNF ligands interact with their respective receptors. TACI and BCMA are unable to mediate the survival activity of BlyS, and the interaction of BAFF-R with neutrokine-alpha was recently determined to be important to peripheral B-cell survival. This highlights the ability of the unique surface of BlyS to interact differently with several receptors.

In summary, the structure of neutrokine-alpha has revealed a distinctive binding groove formed by adjacent monomers within the trimer that permits the cytokine to discriminate among closely related receptors. The floor of the groove seems to harbor shared receptor-binding elements that permit recognition of the three receptors TACI, BCMA and BAFF-R, whereas variations on the outer rims of the groove confer specificity to the interaction. This model, supported by evidence obtained using SELDI affinity mass spectrometry, provides a basis for understanding cytokine receptor-binding specificity and the unique regulation of immune function by neutrokine-alpha. We now have a model that explains both cross-reactivity and specificity. By targeting areas that are implicated in receptor discrimination, developing drugs that can selectively modulate the immunoregulatory functions of neutrokine-alpha should be possible. In particular, a drug which binds to or fits into the groove is useful for selectively modulate the immunoregulatory functions of neutrokine-alpha. Furthermore, a drug that binds to or fits into a portion of the surface of a monomer, wherein said surface is involved in trimerization of neutrokine-alpha monomers, would be useful for modulating the effects of neutrokine-alpha.

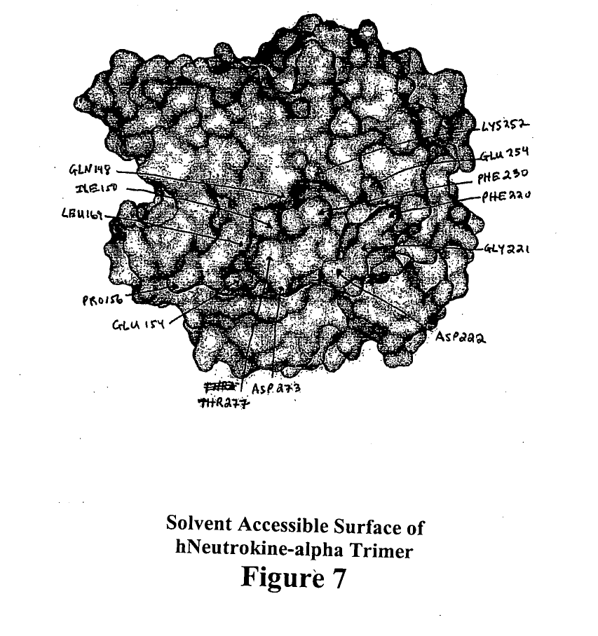

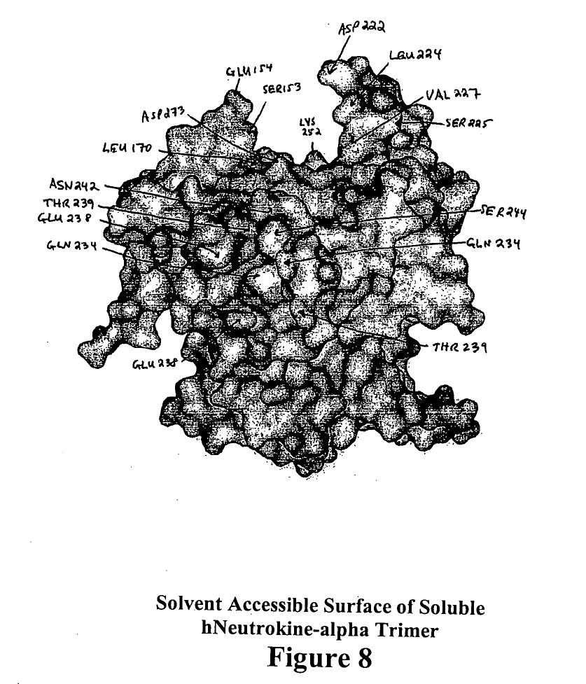

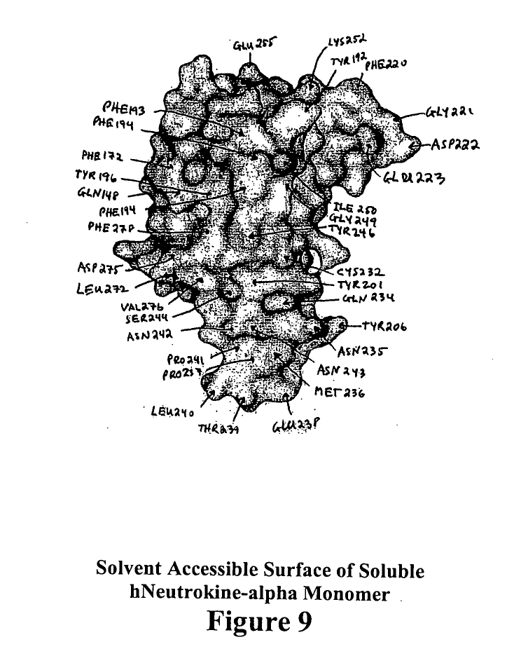

Representations of the major groove on the surface of the neutrokine-alpha protein are provided in FIGS. 7 and 8. Representation of the surface of neutrokine-alpha involved in trimerization is shown in FIG. 9.

Visualiztion of Protein Structure

Although diagrams, such as those in the Figures herein, are useful for visualizing the three dimensional structure of a Neutrokine-alpha protein, a computer program which allows for stereoscopic viewing of the molecule is contemplated as preferred. This stereoscopic viewing, or “virtual reality” as those in the art sometimes refer to it, allows one to visualize the structure in its three dimensional form from every angle in a wide range of resolution, from macromolecular structure down to the atomic level. The computer programs contemplated herein also allow one to change perspective of the viewing angle of the molecule, for example by rotating the molecule. The contemplated programs also respond to changes so that one may, for example, delete, add, or substitute one or more images of atoms, including entire amino acid residues, or add chemical moieties to existing or substituted groups, and visualize the change in structure.

Other computer based systems may be used; the elements being: (a) a means for entering information, such as orthogonal coordinates or other numerically assigned coordinates of the three dimensional structure of a Neutrokine-alpha protein; (b) a means for expressing such coordinates, such as visual means so that one may view the three dimensional structure and correlate such three dimensional structure with the atomic composition of the Neutrokine-alpha protein, such as the amino acid composition; (c) optionally, means for entering information which alters the composition of the Neutrokine-alpha protein expressed, so that the image of such three dimensional structure displays the altered composition.

Once the coordinates are entered into the computer program, one easily displays the three dimensional Neutrokine-alpha protein representation on a computer screen. In one embodiment, the computer system for display is a SGI Octane (San Diego, Calif.). For stereoscopic viewing, one may wear eyewear (Crystal Eyes, SGI) which allows one to visualize the Neutrokine-alpha protein in three dimensions stereoscopically, so one may turn the molecule and envision molecular design.

Several additional, publically and commercially available software programs can be used according to the present invention. Such programs include WHATIF, Sybyl, Insight II, and RasMol (Sayle and Milner-White, “RasMol: Biomolecular graphics for all,” Trends Biochem. Sci. 20:374 (1995)).

Any portion of the Neutrokine-alpha protein may be visualized.

Other preferred characteristics of the three dimensional structure of a Neutrokine-alpha protein, or portion thereof, may be visualized and include lipophilic potential, electrostatic potential, hydrogen bonding ability, local curvature, distance, van der Waals surface, Connolly surface, and solvent accessible surface.

Use of the Coordinates to Determine the Three-Dimensional Structures of Other Neutrokine-Alpha Proteins

Because a Neutrokine-alpha protein may crystallize in more than one crystal form, the structure coordinates of hNeutrokine-alpha protein, or portions thereof, as provided in Table 2, are particularly useful to solve the structure of those other crystal forms of hNeutrokine-alpha or of other Neutrokine-alpha proteins. The coordinates may also be used to solve the structure of Neutrokine-alpha mutants, of a co-complex comprising a neutrokine-alpha protein and one or more small molecules, peptides, or proteins, or of the crystalline form of any other protein with significant amino acid sequence homology to any functional domain of Neutrokine-alpha. Alternatively, the coordinates of hNeutrokine-alpha, or portions thereof, may be used to determine the three-dimensional structure of a Neutrokine-alpha protein of another animal.

One aspect of the present invention that may be employed for this purpose is molecular replacement. In this method, the unknown crystal structure, whether it is another crystal form of hNeutrokine-alpha, a non-human Neutrokine-alpha protein, a Neutrokine-alpha mutant, or a Neutrokine-alpha co-complex, or the crystal of some other protein with significant amino acid sequence homology to any functional domain of Neutrokine-alpha, may be determined using the hNeutrokine-alpha structure coordinates of this invention as provided in Table 2. This method will provide an accurate structural form for the unknown crystal more quickly and efficiently than attempting to determine such information ab initio.

A second aspect of the present invention that may be employed for determining the three-dimensional structure of a Neutrokine-alpha protein, as described above, includes the manual manipulation of the coordinates for hNeutrokine-alpha comprising the coordinates of Table 2, or a portion thereof. In particular, the coordinates are manipulated so that the coordinates of hNeutrokine-alpha, or a portion thereof, are converted into coordinates that encode the three-dimensional structure of a non-human Neutrokine-alpha protein, a Neutrokine-alpha mutant, or a Neutrokine-alpha co-complex, or the crystal of some other protein with significant amino acid sequence homology to any functional domain of Neutrokine-alpha. Preferably, the resulting coordinates encode the three-dimensional structure of a non-human Neutrokine-alpha protein or a Neutrokine-alpha mutant. The method as described comprises the steps of a) displaying the three-dimensional structure of hNeutrokine-alpha using a suitable computer system and a suitable computer program; and b) modifying the three-dimensional structure of hNeutrokine-alpha, thereby producing a three-dimensional structure of a non-human Neutrokine-alpha protein, a Neutrokine-alpha mutant, or a Neutrokine-alpha co-complex, or the crystal of some other protein with significant amino acid sequence homology to any functional domain of Neutrokine-alpha. Said three-dimensional structure of a non-human Neutrokine-alpha protein, a Neutrokine-alpha mutant, or a Neutrokine-alpha co-complex, or the crystal of some other protein with significant amino acid sequence homology to any functional domain of Neutrokine-alpha has one or more atoms or amino acid residues are added, deleted, or modified, compared to hNeutrokine-alpha. The method optionally further comprises a step of using a suitable energy minimization program to minimize the energy of the structure of the modified.

Another aspect of the present invention is a method of determining the structure of a Neutrokine-alpha protein, or portion thereof, complexed with a Neutrokine-alpha receptor, or portion thereof. A suitable Neutrokine-alpha receptor includes BCMA, TACI, or BAFF-R. The structure of the Neutrokine-alpha receptor is determined based on homology modeling to a the known structure of a related receptor, such as TNF-R or DR5. The amino acid composition of the Neutrokine-alpha receptors are known.

Use of Three Dimensional Structure to Design Biologically Active Molecules

Another aspect of the present invention is a method of designing a biologically active molecule that binds to a Neutrokine-alpha protein. Another aspect of the present invention is a method of screening for a biologically active compound that binds to a Neutrokine-alpha protein. The three dimensional structure of a Neutrokine-alpha protein, as provided herein, permits the screening of known molecules and/or the designing of new molecules which bind to a Neutrokine-alpha protein via the use of computerized evaluation systems. For example, computer modeling systems are available in which the sequence of the coordinates of a Neutrokine-alpha protein may be input. Thus, a machine readable medium may be encoded with data representing the coordinates, or a portion thereof, listed in Table 2. The computer then generates structural and/or physicochemical details of a site on the Neutrokine-alpha protein into which a test compound should bind, thereby enabling the determination of the complementary structural details of said test compound.

More particularly, the design of a compound that binds to or inhibits a Neutrokine-alpha protein, in particular hNeutrokine-alpha or a homologue thereof, according to this invention generally involves consideration of two factors. First, said compound must be capable of physically and structurally associating with a Neutrokine-alpha protein. Non-covalent molecular interactions important in the association of said compound with a Neutrokine-alpha protein include hydrogen bonding, van der Waals, hydrophobic, ionic, dipole-dipole, and π-cation interactions. In another embodiment, covalent molecular interactions may be important for the association of said compound with a neutrokine-alpha protein.

Second, the compound must be able to assume a conformation that allows it to associate with a Neutrokine-alpha protein. Although certain portions of the compound will not directly participate in this association with a Neutrokine-alpha protein, those portions may still influence the overall conformation of the molecule. This, in turn, may have a significant impact on potency. Such conformational requirements include the overall three-dimensional structure and orientation of the chemical entity or compound in relation to all or a portion of a binding site on a Neutrokine-alpha protein, or the spacing between functional groups of a compound comprising several chemical entities that directly interact with a Neutrokine-alpha protein.

In one embodiment of the invention, the molecule that is identified or designed according to the methods disclosed herein is a small molecule. In another embodiment of the invention, the molecule that is identified or designed according to the methods disclosed herein is peptide or peptide-mimetic. In a particular embodiment, the molecule that is identified or designed according to the methods disclosed herein is a peptide or peptide-mimetic that has alpha-helical character. In another embodiment of the invention, the molecule that is identified or designed according to the methods disclosed herein is a molecule which binds to or fits into the site in which the citrate molecule is located according to the crystal structure disclosed herein. In another embodiment of the invention, the molecule that is identified or designed according to the methods disclosed herein is a molecule which binds to or fits into the site in which the hydrated magnesium ion is located according to the crystal structure disclosed herein.

Identification of a Molecule that Binds to a Neutrokine-Alpha

There are a number of well-known processes that can be employed to identify a molecule which binds to a Neutrokine-alpha protein. Any number of processes which are known in the art can be employed to identify a molecule which binds to or fits into a site on the neutrokine-alpha protein. In general, a computational method of identifying a molecule according to the present invention is preferred. The particular aspects of computational drug design are well known in the art.

According to the present invention, a NMR-based process may be used to identify a molecule that fits into or binds to a site on the neutrokine-alpha protein. Such methods are known in the art. See, e.g., van Dongen, M., et al. “Structure-based screening and design in drug discovery,” Drug Discov. Today 7:471-478 (2002); Jahnke, W. “Spin labels as a tool to identify and characterize protein-ligand interactions by NMR spectroscopy,” Chembiochem. 3:167-173 (2002); Pochapsky, S. S. and Pochapsky, T. C. “Nuclear magnetic resonance as a tool in drug discovery, metabolism and disposition,” Curr. Top. Med. Chem. 1:427-41 (2001); Sem, D. S. and Pellecchia M. “NMR in the acceleration of drug discovery,” Curr. Opin. Drug Discov. Devel. 4:479-92 (2001); Diercks, T., et al., “Applications of NMR in drug discovery,” Curr. Opin. Chem. Biol. 5:285-91 (2001); Stockman, B. J., et al., “Screening of compound libraries for protein binding using flow-injection nuclear magnetic resonance spectroscopy,” Methods Enzymol. 338:230-46 (2001); Peng, J. W., et al., “Nuclear magnetic resonance-based approaches for lead generation in drug discovery,” Methods Enzymol. 338:202-30(2001); Hicks, R. P. “Recent advances in NMR: expanding its role in rational drug design,” Curr. Med. Chem. 8:627-50(2001); Hajduk, P. J., et al., “NMR-based screening in drug discovery,” Q. Rev. Biophys. 32:211-40 (1999).

According to the present invention, a screening process may be used to identify a molecule which binds to a Neutrokine-alpha protein. For example, a process which utilizes monoclonal antibody technology can be used to screen for a molecule which binds to a site on a Neutrokine-alpha protein. A monoclonal antibody that binds to a Neutrokine-alpha protein can be used in this process. For example, using an assay system comprising a Neutrokine-alpha protein or model thereof, and a monoclonal antibody which binds to Neutrokine-alpha, a molecule can be tested to determine if the molecule binds to the high affinity site. Such screening technology using monoclonal antibodies is known in the art.

Other traditional assays may be used to identify a molecule which binds to Neutrokine-alpha. For example, a radiolabelled ligand which is known to bind to Neutrokine-alpha can be used to screen for additional molecules which bind to Neutrokine-alpha. Such assays are known to those skilled in the art. A molecule, which is not radiolabelled and which is to be tested, is added to the assay system. After a certain equilibration period, the assay system is tested to determine the amount of radioactivity remaining, i.e., the amount of tritiated compound that is still bound to Neutrokine-alpha. The higher the amount of radioactivity, the lower the affinity of the tested molecule, as can be calculated using known relationships, as disclosed in, e.g., Cheng, Y. and Prusoff, W. H. “Relationship between the inhibition constant (Kᵢ) and the concentration of inhibitor which causes 50 percent inhibition (15₀) of an enzymatic reaction,” Biochem. Pharmacol. 22:3099-3108 (1973).

A high throughput screening process can be employed to identify a molecule which binds to Neutrokine-alpha. Such high throughput screening processes are known.

Other processes suitable for identifying a molecule which fits into or binds to the high affinity site may utilize the atomic coordinates to the high affinity site.

According to the present invention, a molecular docking process can be employed to identify a molecule which binds to Neutrokine-alpha. Such docking processes are known in the art. See, e.g., Martin, Y. C., J. Med. Chem. 35:2145-2154 (1992); Halperin, I., Proteins 47:409-43 (2002); Perez, C. et al., J. Med. Chem. 44:3768-85 (2001); Chen et al., Proteins 43:217-26 (2001).

The molecular docking process allows the molecule to be tested as a flexible molecule or as a rigid molecule. When a molecule is tested as a flexible molecule, the three-dimensional conformation of the molecule is subject to change during the process of docking. See, e.g., Anderson, et al., Chem. Biol. 8:445-57(2001). Alternatively, the molecule may be docked as a rigid molecule, wherein the three-dimensional conformation of the molecule is fixed. The three-dimensional conformation of the molecule may be fixed based on a number of factors known in the art, including, but not limited to, an energy-minimization calculation or a known crystal structure of said molecule. Alternatively, the three-dimensional conformation may be fixed based on the structure of a known Neutrokine-alpha ligand.

The molecular docking process may allow for the conformation of the site on the neutrokine-alpha protein to be flexible. That is, the exact conformation of the Neutrokine-alpha protein may change during the docking process. The exact conformation of the side chains of the amino acids may change due to the molecule binding to or fitting into the site on the neutrokine-alpha protein. A change in the conformation of the protein upon binding of a molecule is a known phenomenon and is often referred to as “induced fit.” Several docking algorithms known in the art allow for flexibility in the site on the neutrokine-alpha protein.

Alternatively, the molecular docking process may allow for the site on the neutrokine-alpha protein to be rigid. Setting the site on the neutrokine-alpha protein to be rigid has an advantage of permitting the molecular docking process to be performed more quickly.

According to the present invention, a molecule which is used in the above identifying process may be selected from any number of sources. Screening a library, or database, of molecules is a useful method. Structure-based processes of screening one or more libraries of molecules are known in the art. See, e.g., Diller et al., Proteins 43:113-24 (2001). For example, a user may randomly select a molecule from a database. A computer may randomly select a molecule from a database. A number of commercially available databases, or libraries, are available, including, but not limited to, Cambridge Structural Database (Cambridge Crystallographic Data Centre); Ligand™ (Databases of Chemical Compounds and Reactions in Biological Pathways; http://www.genome.ad.jp/ligand/); World Drug Index; National Cancer Institute databases (see http://dtp.nci.nih.gov/docs/3d_database/structural_information/structural_data.html); TRIAD™ (Paul A. Bartlett, University of California, Berkley); Unity™ (Tripos, Inc.) and others.

A user may build a molecule according to the user's predetermined criteria and then use that molecule in the identifying process.

A user or computer may apply one or more initial filters to the database, or library, of compounds, thereby producing a smaller and more focused database. Such filtering methods are known in the art. For example, a user or computer may apply “Lipinski's Rules” to remove compounds which are believed to be poor drug candidates. See, e.g., Lipinski, C. A., J. Pharmacol. Toxicol. Methods 44:235-249 (2000). A molecule, selected from the resulting database containing molecules that are believed to be more drug like, is then used in the above identifying process.

Additionally, a user or computer may apply one or more filters to the molecules selected for testing so that one or more chemical groups are either present in or absent from the molecules selected. For example, a user or computer may select molecules which contain at least one or two aromatic rings. Alternatively, a user or computer may select molecules which contain one or more negatively charged functional groups. Other parameters which may be used to filter molecules comprise the presence or absence of one or more phenyl rings; one or more pyridine rings; and one or more aromatic rings.

Additionally, a user or computer may apply a filter which selects a molecule based on its ADME properties. ADME properties refer to absorption, distribution, metabolism, and excretion properties of a molecule. For a molecule to be selected as a drug candidate to be developed into a drug, the ADME properties of the molecule should be acceptable, as is known in the art. See, e.g., Selick, H. E. et al., “The emerging importance of predictive ADME simulation in drug discovery,” Drug Discov. Today 7:109-116 (2002).

Alternatively, a user may construct a molecule using a software program and then subject said molecule to a docking algorithm. Such a process may utilize the user's knowledge and intuition regarding the identification of biologically active molecules.

Certain of the processes described herein as being suitable to be employed to identify a molecule which binds to a Neutrokine-alpha protein may also be referred to as processes of virtual screening. Virtual screening is known in the art and is as described more fully in Walters et al., “Virtual screening—an overview,” Drug Discov. Today 3:160-178 (1998). It is understood that a process of virtual screening can be employed to identify a molecule which binds to a Neutrokine-alpha protein.

A number of software programs can be employed to identify a molecule that binds to or fits into a site on the neutrokine-alpha protein. Such programs include, but are not limited to, Dock™ (Ewing et al., J. Comput. Aided Mol. Des. 15:411-28 (2001)); AutoDock™ (Scripps Research Institute; Morris, G. M., et al., J. Comp. Chem. 19: 1639-1662 (1998)); Slide™ (Leslie Kuhn of Michigan State University); FlexX™ (Tripos, Inc.); FlexE (Claussen, H., et al., J. Mol. Biol. 308:377-95 (2001); ICM™ (Internal Coordinate Mechanics); QXP™; Ecepp/Prodock™; Pro_LEADS™; Hammerhead™; FLOG™; GOLD™; LUDI™; GREEN™; X-Ligand™ (Accelrys, Inc.); Glide (Schrödinger, Inc.); and Galaxy™ (AM Technologies, Inc.).

According to the present invention, a genetic algorithm may be employed to identify or design a molecule which binds to a Neutrokine-alpha protein. Such genetic algorithms are known in the art. See, e.g., Pegg, S. C., et al., J. Comput. Aided Mol. Des. 15:911-33 (2001).

Additional, suitable processes which can be employed to identifying or designing a molecule which binds to a Neutrokine-alpha protein include those processes disclosed in U.S. Pat. Nos. 6,389,378; and 6,308,145.

Design of a Molecule that Binds to a Neutrokine-Alpha

There are a number of well-known processes that can be employed to design a molecule which binds to a Neutrokine-alpha protein. Any number of processes which are known in the art can be employed to design a molecule which binds to a Neutrokine-alpha protein. In general, computational methods of designing a molecule according to the present invention are preferred. The particular aspects of computational drug design are well known in the art.

According to the present invention, a NMR-based process of designing a molecule which binds to a Neutrokine-alpha protein can be used. For example, a method commonly known as “SAR by NMR” can be used to design a molecule. SAR by NMR is described in detail in Shuker, S. B., et al., “Discovering High-Affinity Ligands for Proteins: SAR by NMR,” Science 274:1531-1534 (1996) and in U.S. Pat. Nos. 5,989,827 and 5,891,643. Briefly and in general, the SAR by NMR method comprises using ¹⁵N- and ¹H-amide chemical shift changes of the protein upon ligand binding to determine binding location and orientation. The process is repeated with a second ligand in order to identify a second ligand which binds to portion of the protein which is spatially near the binding location of the first ligand. Upon identification of two ligands which bind closely on the protein, a molecule can be designed, said molecule comprising both identified ligands, or portions thereof, and a linker moiety connecting said ligands, or portion thereof.

According to a SAR by NMR process to be used according to the present invention, a ¹⁵N-labeled Neutrokine-alpha protein is prepared according to known methods. The ¹⁵N-labeled Neutrokine-alpha protein is used in the SAR by NMR process, along with various small molecules which are thought to be capable of binding to the high affinity site. Examples of such small molecule include: benzene, pyrimidine, acetylcholine, amino acids, dipeptides, each of which are optionally substituted. Using the identified ligands, a molecule is designed incorporating a molecule, or fragment thereof, which binds to or fits into the right subsite, and a molecule, or fragment thereof, which binds to or fits into the left subsite. Said designed molecule also incorporates a linker moiety which connects the two identified molecules, or fragments thereof. Such linker moieties may be any suitable functional group or chemical moiety.

Another suitable process which can be employed to design a molecule which binds to a Neutrokine-alpha protein comprises modifying a known ligand which binds to a Neutrokine-alpha protein, and testing said modified ligand to determine if said modified ligand inhibits, modulates, or regulates said Neutrokine-alpha protein. A starting compound may contain a phenyl ring, for example. A suitable modification may include making a similar compound with a bromine on the phenyl ring. When the bromo compound is made, it can be tested to determine if it inhibits, modulates, or regulates a Neutrokine-alpha protein. The compound may further be modeled using a molecular modeling program and docked onto a model of a Neutrokine-alpha protein.

Other processes suitable for designing a molecule which binds to a Neutrokine-alpha protein may utilize the atomic coordinates to the high affinity site.

According to the present invention, a fragment-based design process may be employed to design a molecule which binds to a Neutrokine-alpha protein. In general, a fragment-based process determines which molecular fragments are most likely to have a high affinity for certain portions of the protein. Fragments used may be individual atoms, small fragments of molecules such as a hydroxyl radical, or small molecules such as a water molecule. The process by which the fragments are determined to have a high affinity can vary and can included processes using empirical force fields, random distribution of fragments, Monte Carlo-based approach, a molecular docking process, or other processes. After the given algorithm determines the types of fragments with high affinity for the protein and the location on the protein to which said fragments bind, an overall three-dimensional picture of fragments is produced. All or some of the fragments are then joined to form a molecule, said molecule being one that binds to or fits into the high affinity site. The fragments may be joined to form a molecule using an automated process or a user-based process. In an automated process, a computer determines which chemical linkers are used to connect the fragments. In a user-based process, a user determines which chemical linkers are used to connect the fragments.

As described above, in using a fragment-based design process, any number of molecular fragments can be used, such as an oxygen atom, a hydroxyl radical, or a water molecule.

Additional, suitable processes which can be employed to design a molecule according to the present invention include those processes disclosed in U.S. Pat. Nos. 6,226,603; and 5,854,992.

Alternatively, a template-based process of designing a molecule can be employed. In a template-based process, a first molecule, which is known to bind to a Neutrokine-alpha protein, is used as a template to design or identify a second molecule which binds to said protein. In this process, the first molecule, herein referred to as the known ligand, may be positioned in a binding site by, for example, using a molecular docking process, which may be either automated or user-controlled. The known ligand may optionally be subjected to an energy minimization process within the binding site. By subjecting the known ligand to such an energy minimization process, the user may determine the most probable three-dimensional conformation of the known ligand when bound to the protein.

When the known ligand is positioned in the binding site, the known ligand may be used in an automated process to design a molecule. For example, an algorithm which systematically adds a chemical group to or deletes a chemical group from the known ligand can be employed. After the change in the structure of the known ligand, the effect of the change can be determined by computationally determining the interaction between the protein and the modified ligand. If the interaction between the modified ligand and the protein is greater (i.e., higher affinity) than the interaction between the known ligand and the protein, then the structural modification is determined to beneficial. Provided that the modified ligand binds to the binding site as required herein, the modified ligand is thus determined to be a molecule as designed according to the present invention.

Alternatively, when the known ligand is positioned in the binding site, the known ligand may be used in a manual process to design a molecule. For example, a user may add a chemical group to or delete a chemical group from the known ligand. Such changes can be made using the knowledge or intuition of the user in conjunction with the teachings herein. After the change in the structure of the known ligand, the effect of the change can be determined by computationally determining the interaction between the protein and the modified ligand. If the interaction between the modified ligand and the protein is greater than the interaction between the known ligand and the protein, then the structural modification is determined to beneficial. Provided that the modified ligand binds to the binding site as required herein, the modified ligand is thus determined to be a molecule as designed according to the present invention.

A number of software programs can be employed to design a molecule which binds to a Neutrokine-alpha protein. Such programs include, but are not limited to, the following: MCSS™ (Accelrys, Inc.); LUDI™ (Accelrys, Inc.); SMoG™ (Harvard University); SPROUT™ (University of Leeds); RASSE™ (See J. Chem. Inf. Comput. Sci. 36:1187-1196 (1996)); MCSS/Hook™ (Accelrys, Inc.); Cerius2™ (Accelrys, Inc.); CAVEAT™ (Lauris et al., J. Comp.-Aided Mol. Design 8:51-66 (1994); LeapFrog™ (Tripos, Inc.); GRID™ (Oxford University;: Goodford, P., et al., J. Med. Chem. 36:148-56(1993)); and GroupBuild (Vertex, Inc.).

A further aspect of the present invention is directed to employing a pharmacophore-based process to identify or design a molecule which binds to a Neutrokine-alpha protein. Pharmacophore-based processes are known in the art. See, e.g., Kurogi and Guner, “Pharmacophore modeling and three-dimensional database searching for drug design using catalyst,” Curr. Med. Chem. 8:1035-1055 (2001). Generally, the process involves the determination of the optimal chemical functional groups that are required in a molecule to bind to or fit into a certain target. The pharmacophore will also usually specify the two-dimensional or three-dimensional relationship among the functional groups. Using the pharmacophore, one may identify or design a molecule which contains all or most of the functional groups specified by the pharmacophore. Having successfully identified or designed said molecule, one may optionally further test said molecule in a computational manner. One may further synthesize or prepare said molecule. Having synthesized and tested said molecule, one may test said molecule in one or more biological assays, as described below.

The methods described herein can be employed to design or identify compounds that bind to a Neutrokine-alpha protein.

In the above processes which utilize the three-dimensional coordinates of a Neutrokine-alpha protein, whether for identifying or designing a molecule according to the present invention, said processes may utilize one or more general processes to determine whether said molecule binds to or fits into the site on the neutrokine-alpha protein. For example, some of processes described herein may utilize a molecular mechanics based process to determine the interaction between said molecule and said site on the neutrokine-alpha protein. Alternatively, certain processes described herein may utilize a semi-empirical based process, such as AM1 force field, to determine the interaction between said molecule and said site on the neutrokine-alpha protein. Certain processes described herein may utilize a quantum mechanical based process, such as GAMESS or GAUSSIAN, to determine the interaction between said molecule and said site on the neutrokine-alpha protein. Certain processes described herein may utilize a molecular dynamics based process to determine the interaction between said molecule and said site on the neutrokine-alpha protein. Such processes are known in the art. See, e.g., Halperin, I., et al., “Principles of docking: An overview of search algorithms and a guide to scoring functions.” Proteins 47:409-43 (2002).

For example, according to the present invention, the major groove on the surface of the Neutrokine-alpha trimer has herein been identified as a target for drug discovery and design. A variety of amino acids comprise the groove as described herein. For example, GLU223 forms part of the wall of the groove and prominently displays its terminal carboxyl group. The presence of this negatively charged group of GLU223 can be used to design or identify a compound that will bind to the pocket. Said compound can incorporate a positively charged functional group to interact with the negatively charged carboxyl group of GLU223. Such positively charged groups are well known in the art and include, but are not limited to, amino, guanidinium, histidine, and pyridyl. Other amino acids that form the major groove, or other depressions or cavities, can be similarly identified and used to design or identify, according to the present invention, a compound that binds to a Neutrokine-alpha protein.

Second, the compound must be able to assume a conformation that allows it to associate with a Neutrokine-alpha protein. Although certain portions of the compound will not directly participate in this association with a Neutrokine-alpha protein, those portions may still influence the overall conformation of the molecule. This, in turn, may have a significant impact on potency. Such conformational requirements include the overall three-dimensional structure and orientation of the chemical entity or compound in relation to all or a portion of a binding site on a Neutrokine-alpha protein, or the spacing between functional groups of a compound comprising several chemical entities that directly interact with a Neutrokine-alpha protein.

The potential inhibitory or binding effect of a chemical compound on a Neutrokine-alpha protein may be analyzed prior to its actual synthesis and testing by the use of computer modeling techniques. If the theoretical structure of the given compound suggests insufficient interaction and association between said compound and a Neutrokine-alpha protein, synthesis and testing of the compound is obviated. However, if computer modeling indicates a strong interaction, the molecule may then be synthesized and tested for its ability to bind to and/or inhibit a Neutrokine-alpha protein using a suitable assay. In this manner, synthesis of inoperative compounds may be avoided or minimized.

As is known in the art, a number of methods are available to determine whether a compound will interact with a protein. Such methods include general molecular mechanics calculations, semi-empirical methods such as AM1, and quantum mechanical or ab initio calculations such as Jaguar™, Hondo™, Gamess™, and Gaussian™. Another suitable method includes Hint™ (eduSoft).

An inhibitory or other binding compound of a Neutrokine-alpha protein, or portion thereof, may be computationally evaluated and designed by means of a series of steps in which chemical entities or fragments are screened and selected for their ability to associate with the individual binding pockets or other areas of the Neutrokine-alpha protein.

According to the methods of the present invention, one may design or identify a compound that inhibits or reduces that activity of a Neutrokine-alpha protein. In particular, a compound that inhibits or reduces that activity of a Neutrokine-alpha protein may be a compound that binds to the surface of said Neutrokine-alpha protein and inhibits or reduces the protein's ability to bind to or activate a receptor, such as TACI, BAFF-R, and BCMA. Alternatively, a compound that inhibits or reduces that activity of a Neutrokine-alpha protein may be a compound that binds to the surface of a monomer of said Neutrokine-alpha protein and inhibits or reduces the ability of said monomer to form trimers of Neutrokine-alpha. Alternatively, a compound that inhibits or reduces that activity of a Neutrokine-alpha protein may be a compound that binds to the surface of a trimer of said Neutrokine-alpha protein and inhibits or reduces the ability of said monomer to form dimers of trimers or to form other assemblies of monomers or trimers, of Neutrokine-alpha.

According to the present invention, one may also design or identify a compound that enhances the activity of a Neutrokine-alpha protein. For example, a compound that enhances the activity of a Neutrokine-alpha protein may be a compound that binds to the surface of a monomer of said Neutrokine-alpha protein and increases the ability of said monomer to form trimers of Neutrokine-alpha. Alternatively, a compound that enhances that activity of a Neutrokine-alpha protein may be a compound that binds to the surface of a trimer of said Neutrokine-alpha protein and increases the ability of said monomer to form dimers of trimers or to form other assemblies of monomers or trimers, of Neutrokine-alpha.

By way of example, a starting compound used to design a compound that enhances the activity of a Neutrokine-alpha protein is citric acid. As identified in the crystal structure disclosed herein, a citrate molecule interacts with two monomers of the trimeric form of hNeutrokine-alpha protein. Specifically, the negatively charged carboxylate groups of the citrate molecule interact with the positively charged Arg214, Lys 216, His218, and Lys252. By using the molecular modeling methods as described herein, a new compound that binds to the two monomers of hNeutrokine-alpha can be designed using citrate as a template molecule. A model of the citrate molecule may be modified so that a new molecule forms closer and stronger interactions with certain proximate amino acids such as Glu254 and Lys252. Alternatively, a compound can be designed to interact with Phe220 via a pi-cation, hydrophobic, or aromatic interaction. Energy calculations, e.g., molecular mechanics, Gibbs free energy, HINT™, can be performed using the modified compound compared to citrate. If the interaction energy among the modified compound and the two Neutrokine-alpha monomers is more favorable than the interaction energy among citrate and the two Neutrokine-alpha monomers, then the modified compound is expected to be able to enhance the association of the two monomers.

One skilled in the art may use one of several methods to screen chemical entities or fragments for their ability to associate with a Neutrokine-alpha protein, or portion thereof, and more particularly with one or more individual binding pockets of the a Neutrokine-alpha protein, or portion thereof. This process may begin by visual inspection of, for example, the three dimensional structure of a Neutrokine-alpha protein on a computer screen, based on the atomic coordinates, or portion thereof, in Table 2. Selected fragments or chemical entities may then be positioned in a variety of orientations, or docked, within a binding pocket of a Neutrokine-alpha protein. Docking may be accomplished using software such as Quanta™ and Sybyl™, followed by energy minimization and molecular dynamics with standard molecular mechanics force fields, such as CHARMM™ (Chemistry at HARvard Macromolecular Mechanics) and AMBER™.

Specialized computer programs may also assist in the process of selecting fragments or chemical entities. These include: GRID™; MCSS™ (Multiple Copy Simultaneous Search); AUTODOCK™; FlexX™; and DOCK™.

Once suitable chemical entities or fragments have been selected, the chemical entities or fragments can be modeled into a single compound or inhibitor. Assembly may be proceed by visual inspection of the relationship of the fragments to each other on the three-dimensional image displayed on a computer screen in relation to the structure coordinates of the Neutrokine-alpha protein. This would be followed by manual model building using software such as Quanta™, InsightII™, or Sybyl™.

Useful programs to aid one of skill in the art in connecting the individual chemical entities or fragments include: CAVEAT™; MACCS-3D™; and HOOK™.

Instead of proceeding to construct a compound that binds to Neutrokine-alpha in a step-wise fashion one fragment or chemical entity at a time as described above, inhibitory or other Neutrokine-alpha-binding compounds may be designed as a whole, or de novo, using either at least a portion of the coordinates of a Neutrokine-alpha protein or optionally including at least a portion of one or more known inhibitors. Computer programs useful for this method include: LUDI™; LEGEND™; LeapFrog™, and SMoG™ (Harvard University).

In another aspect of the present invention, a library of molecules is searched for one or more compounds that can bind to a Neutrokine-alpha protein, or portion thereof. The library of molecules to be searched can be any library, such as a database (i.e., online, offline, internal, external) which comprises crystal structures, coordinates, chemical configurations or structures of molecules, compounds, or drugs (referred to collectively as to be assessed or screened for their ability to bind to a Neutrokine-alpha protein). For example, databases for drug design, such as the Cambridge Structural Database (CSD), which includes about 100,000 molecules whose crystal structures have been determined or the Fine Chemical Director (FCD) distributed by Molecular Design Limited (San Leandro, Calif.) can be used. [CSD: Allen et al., Acta Crystallogr. Section B 35:2331 (1979)]. In addition, a library, such as a database, biased to include an increased number of members which comprise indole rings, hydrophobic moieties and/or negatively-charged molecules can be used.

According to the present invention, any portion of the structure of a Neutrokine-alpha protein may be used to design a compound that binds to or inhibits a Neutrokine-alpha protein. Preferred portions of the structure include amino acid residues that define a pocket or groove on the surface of the Neutrokine-alpha protein. One set of preferred residues comprises Q148, I150, A151, D152, S153, E154, L169, L170, F172, L201 T202, D203, I270, S271, L272, D273, G274, and D275 of the A monomer together with T190, Y192, A207, G209, H210, L211, Q213, R214, K216, H218, F220, D222, E223, L224, L226, V227, T228, L229, F230, R231, 1233, A251, K252, and E254 of the C monomer. Thus, a preferred aspect of the present invention is a method of designing a compound that binds to a Neutrokine-alpha protein, said method comprising the steps of analyzing computationally a compound to determine if said compound binds to a portion of a Neutrokine-alpha protein wherein said portion comprises Q148, 1150, A151, D152, S153, E154, L169, L170, F172, L200, T202, D203, I270, S271, L272, D273, E274, and D275 of the A monomer together with T190, Y192, A207, G209, H210, L211, Q213, R214, K216, H218, F220, D222, E223, L224, L226, V227, T228, L229, F230, R231, I233, A251, K252, and E254 of the C monomer. Preferably, the compound is substantially complementary to the portion of Neutrokine-alpha with respect to polar and lipophilic character of said portion of Neutrokine-alpha.

Other areas of Neutrokine-alpha are suitable targets for designing or identifying a drug that inhibits or binds to Neutrokine-alpha. Such portions of the Neutrokine-alpha include an area selected from the following: 1) an area defined by Q148, I150, A151, D152, S153, E154, L169, L170, F172, L200, T202, D203, I270, S271, L272, D273, E274, and D275 of a first monomer together with T190, Y192, A207, G209, H210, L211, Q213, R214, K216, H218, F220, D222, E223, L224, L226, V227, T228, L229, F230, R231, I233, A251, K252, and E254 of a second monomer; 2) an area defined by Q148, I150, A151, D152, S153, E154, L169, L170, F172, L200, T202, D203, I270, S271, L272, D273, E274, D275, and F278 of a first monomer together with T190, Y192, A207, G209, H210, L211, Q213, R214, K216, H218, F220, D222, E223, L224, L226, V227, T228, L229, F230, R231, I233, A251, K252, L253, E254, and D257 of second monomer; and 3) an area defined by the amino acids from A251 to L229, inclusive.

Another suitable area of a Neutrokine-alpha protein includes an area which comprises amino acids that are within about 30 Å, 25 Å, 20 Å, 15 Å, 10 Å, or 5 Å of an amino acid selected from the group consisting of THR141-LEU285. In one embodiment, the are comprises amino acids within about 10 Å or 5 Å of an amino acid selected from the group consisting of THR141-LEU285.

An additional aspect of the present invention is a method of designing a compound that mimics the biological activity of a Neutrokine-alpha protein. Said method comprises identifying or designing a compound based on a three-dimensional structure of a Neutrokine-alpha protein, so that said compound resembles at least partially structurally and chemically similar to at least a portion of said Neutrokine-alpha protein. The method further comprises synthesizing and testing said compound for biological activity, preferably for Neutrokine-alpha-like activity.

An additional aspect of the present invention is a method of designing a compound that is structurally and chemically similar to a Neutrokine-alpha protein, or portion thereof, wherein said method comprises analyzing said compound to determine if said compound is structurally and chemically similar to a Neutrokine-alpha protein, or portion thereof. According to the present invention, the compound is analyzed using the three dimensional structure of a Neutrokine-alpha protein or portion thereof.

One advantage of the present method is that the method allows one to determine potentially if a compound will have biological activity before synthesizing and assaying said compound. Thus, large numbers of compounds can be analyzed using computational means. Preferred biological activities are either Neutrokine-alpha-inhibitor activity or Neutrokine-alpha-like activity.

Various computational analyses are necessary to determine whether a molecule or portion thereof is sufficiently similar to all or part of a three-dimensional structure of a Neutrokine-alpha protein. Such analyses may be carried out with computer programs that are well known in the art, such as QUANTA. In particular, the Molecular Similarity module of QUANTA is used.

The Molecular Similarity module permits the comparisons between different structures, different conformations of the same structure, and different parts of the same structure. The procedure used in Molecular Similarity to compare structures comprises the following four steps: 1) input the structures to be compared; 2) define the atom equivalence in the structures; 3) perform a fitting, i.e., superposition, operation; and 4) analyze the results.

In the above steps, one structure is identified as the target, i.e., the fixed structure; all the remaining structures are working structures, i.e., moving structures. Since atom equivalency within QUANTA is defined by user input, root mean square deviation (RMSD) values can be determined in a number of ways. When comparing the structures of peptides, using the C_{α} backbone carbons provides preferable results.

According to the methods of the present invention, a compound will be chemically similar to a Neutrokine-alpha protein, or portion thereof, if said compound resembles said Neutrokine-alpha protein or portion thereof in terms of one of more of the following chemical characteristics: lipophilicity; logP; hydrophilicity; polarity; aromatic character; hydrogen bonding character; and presence of charged moieties. Thus, after determining that a compound is structurally similar to a Neutrokine-alpha protein, or portion thereof, one may determine if said compound is chemically similar to the Neutrokine-alpha protein, or portion thereof. Comparisons may be made based on the presence or absence of chemical functional groups. Additionally, comparisons may be made based on the overall lipophilicity of the compound compared to the overall lipophilicity of the Neutrokine-alpha protein, or portion thereof.

A preferred aspect of the present invention is identifying or designing a compound that mimics, antagonizes, or inhibits Neutrokine-alpha activity, wherein said compound is a cyclic or rigid peptide that is structurally and chemically similar to a Neutrokine-alpha protein or portion thereof.

Another preferred aspect of the present invention designing a compound that mimics, antagonizes, or inhibits Neutrokine-alpha activity, wherein said compound is a cyclic or rigid peptidomimetic that is structurally and chemically similar to a Neutrokine-alpha protein or portion thereof.

In an additional embodiment, the present invention is directed to a method of designing or identifying a drug which fits into or binds to a groove on the surface of a neutrokine-alpha protein trimer. Preferably the groove is as described above, although other grooves are included within the scope of the invention. In particular, the groove is created by loops from two adjacent monomers. One wall of the groove contains loop DE with some residues of loops aa′ and GH, and the other wall of the groove contains loops EF, Aa, and a′A″. The deepest portion of the groove consists primarily of beta-strands D, E, and F. Residues with surface accessible side chains are ALA207, LEU211, GLN213, and ARG214 from strand D; THR228, LEU229, PHE230, ARG231, and ILE233 from strand E; and ALA251, LYS252, LEU253, GLU254, and ASP257 from strand F. Any of the methods described herein can be used to identify or design a drug that binds to or fits into said groove.

In another embodiment of the present invention, the binding affinity of said a molecule designed or identified according to the present invention is determine. The binding affinity can be calculated using computational methods which are known in the art, or can be calculated empirically using assays as described herein or are known in the art.

In another embodiment, the present invention is directed to a method of designing or identifying a compound which binds to or fits into the hydrated magnesium ion binding site. A compound which binds to or fits into the hydrated magnesium ion binding site is able to disrupt the trimerization of the monomers and thus would inhibit, decrease, or modulate the activity of neutrokine-alpha.

Any portion of the three dimensional structure of a Neutrokine-alpha protein may be used to design, or screen for, a compound that is structurally and chemically similar to said Neutrokine-alpha protein or portion thereof. Preferred portions of the three-dimensional structure of a Neutrokine-alpha protein for use in the aforementioned methods include one or more of the β-sheets a, a′, A, A′, B, B′, C, D, E, F, G, and H; one or more of the loops between a and a′; between a and A; between A and A″; between A″ and B′; between B′ and B; between B and C; between C and D; between D and E; between E and F; between F and G; and between G and H. Additionally, portions of each of the aforementioned β-sheets and loops may be used. Particularly preferred portions of the three dimensional structure of a Neutrokine-alpha protein are one or more of β-sheets a, a′, A, and A′; and one or more of loops between a and a′; between a and A; between C and D; between D and E; between E and F; between F and G; and between G and H.

Additionally, combinations of the aforementioned β-sheets and loops may be used to design, or screen for, a compound that is structurally and chemically similar to said Neutrokine-alpha protein or portion thereof. For example, the method of the present invention can be used to design, or screen for, a compound that is similar in shape and chemical attributes to a the overall shape of the D and E β-sheets.

As used herein, with respect to a Neutrokine-alpha protein or analogue thereof, or with respect to a region of a Neutrokine-alpha protein or analogue thereof, the phrase “at least a portion of the three-dimensional structure of” or “at least a portion of” is understood to mean a portion of the three-dimensional surface structure of the Neutrokine-alpha protein, or region of the Neutrokine-alpha protein, optionally including charge distribution and hydrophilicity/hydrophobicity characteristics, formed by at least three, more preferably at least three to ten, and most preferably at least ten contiguous amino acid residues of the Neutrokine-alpha monomer, dimer or trimer. The contiguous residues forming such a portion may be residues which form a contiguous portion of the primary structure of the Neutrokine-alpha molecule, residues which form a contiguous portion of the three-dimensional surface of the Neutrokine-alpha monomer, residues which form a contiguous portion of the three-dimensional surface of the Neutrokine-alpha dimer, residues which form a contiguous portion of the three-dimensional surface of the Neutrokine-alpha trimer, or a combination thereof. Thus, the residues forming a portion of the three-dimensional structure of the Neutrokine-alpha protein need not be contiguous in the primary sequence of the Neutrokine-alpha protein but, rather, must form a contiguous portion of the surface of the Neutrokine-alpha protein. In particular, such residues may be non-contiguous in the primary structure of a single Neutrokine-alpha protein monomer or may comprise residues from different Neutrokine-alpha protein monomers in the dimeric or trimeric form of the Neutrokine-alpha protein. As used herein, the residues forming “a portion of the three-dimensional structure of” a Neutrokine-alpha protein, or “a portion of” a Neutrokine-alpha protein, form a contiguous three-dimensional surface in which each atom or functional group forming the portion of the surface is separated from the nearest atom or functional group forming the portion of the surface by no more than about 40 Å, preferably by no more than about 20 Å, more preferably by no more than about 5-10 Å, and most preferably by no more than about 1-5 Å.

As used herein, the term “X-ray crystallographic co-ordinates” refers to a series of mathematical co-ordinates (represented as “X”, “Y” and “Z” values) that relate to the spatial distribution of reflections produced by the diffraction of a monochromatic beam of X-rays by atoms of a molecule in crystal form. The diffraction data are used to generate electron density maps of the repeating units of a crystal, and the resulting electron density maps are used to define the positions of individual atoms within the unit cell of the crystal.

As will be apparent to those of ordinary skill in the art, the hNeutrokine-alpha structure presented herein, and other three dimensional structures of Neutrokine-alpha proteins determined according to the methods described herein, are independent of their orientation, and that the atomic coordinates listed in TABLE 2 merely represent one possible orientation of the human Neutrokine-alpha structure. It is apparent, therefore, that the atomic coordinates listed in TABLE 2 may be mathematically rotated, translated, scaled, or a combination thereof, without changing the relative positions of atoms or features of the hNeutrokine-alpha structure. Such mathematical manipulations are intended to be embraced herein. Furthermore, it will be apparent to the skilled artisan that the X-ray atomic coordinates defined herein have some degree of uncertainty in location. Accordingly, for purposes of this invention, a preselected protein or peptide having the same amino acid sequence as at least a portion of Neutrokine-alpha is considered to have the same structure as the corresponding portion of Neutrokine-alpha, when a set of atomic co-ordinates defining backbone C_{α} atoms of the preselected protein or peptide can be superimposed onto the corresponding C_{α} atoms for Neutrokine-alpha to a root mean square deviation of preferably less than about 3.0, 2.5, 2.0, 1.5, 1.4, 1.3, 1.2, 1.1, or 1.0 Å, and most preferably less than about 0.95, 0.90, 0.85, 0.80, 0.75, 0.70, 0.65, 0.60, 0.55, or 0.50 Å. In one embodiment, the neutrokine-alpha structure comprises the coordinates shown in Table 2, or a portion thereof. In another embodiment, the neutrokine-alpha structure comprises the coordinates provided in Accession I.D. No.: 1KXG, (deposited Jan. 31, 2002) of the Protein Data Bank, or a portion thereof. (H. M. Berman, et al., The Protein Data Bank. Nucleic Acids Research, 28 pp. 235-242 (2000)). In another embodiment, the neutrokine-alpha structure comprises the coordinates provided in Table 2, or portion thereof, having undergone a routine energy-minimization process. In another embodiment, the neutrokine-alpha structure comprises the coordinates provided in Accession I.D. No.: 1KXG, or portion thereof, having undergone a routine energy-minimization process.

According to the methods of the present invention, the atomic coordinates of a Neutrokine-alpha protein in crystalline form may be used in various ways. When the atomic coordinates of a Neutrokine-alpha protein in crystalline form are used, the entire set of coordinates of the protein, including associated water molecules, citrate molecules, dioxane molecules, and magnesium ions, may be used. Alternatively, a portion of the atomic coordinates of Neutrokine-alpha in crystalline form may be used according to the methods of the present invention. A portion of the coordinates that may be used according to the present invention include coordinates that comprise, or alternatively consist of, the coordinates of an amino acid sequence selected from the group consisting of residues: T-141 to T-155; V-142 to P-156; T-143 to T-157; Q-144 to I-158; D-145 to Q-159; C-146 to K-160; L-147 to G-161; Q-148 to S-162; L-149 to Y-163; I-150 to T-164; A-151 to F-165; D-152 to V-166; S-153 to P-167; E-154 to W-168; T-155 to L-169; P-156 to L-170; T-157 to S-171; I-158 to F-172; Q-159 to K-173; K-160 to R-174; G-161 to G-175; S-162 to S-176; Y-163 to A-177; T-164 to L-178; F-165 to E-179; V-166 to E-180; P-167 to K-181; W-168 to E-182; L-169 to N-183; L-170 to K-184; S-171 to I-185; F-172 to L-186; K-173 to V-187; R-174 to K-188; G-175 to E-189; S-176 to T-190; A-177 to G-191; L-178 to Y-192; E-179 to F-193; E-180 to F-194; K-181 to 1-195; E-182 to Y-196; N-183 to G-197; K-184 to Q-198; I-185 to V-199; L-186 to L-200; V-187 to Y-201; K-188 to T-202; E-189 to D-203; T-190 to K-204; G-191 to T-205; Y-192 to Y-206; F-193 to A-207; F-194 to M-208; I-195 to G-209; Y-196 to H-210; G-197 to L-211; Q-198 to I-212; V-199 to Q-213; L-200 to R-214; Y-201 to K-215; T-202 to K-216; D-203 to V-217; K-204 to H-218; T-205 to V-219; Y-206 to F-220; A-207 to G-221; M-208 to D-222; G-209 to E-223; H-210 to L-224; L-211 to S-225; I-212 to L-226; Q-213 to V-227; R-214 to T-228; K-215 to L-229; K-216 to F-230; V-217 to R-231; H-218 to C-232; V-219 to 1-233; F-220 to Q-234; G-221 to N-235; D-222 to M-236; E-223 to P-237; L-224 to E-238; S-225 to T-239; L-226 to L-240; V-227 to P-241; T-228 to N-242; L-229 to N-243; F-230 to S-244; R-231 to C-245; C-232 to Y-246; I-233 to S-247; Q-234 to A-248; N-235 to G-249; M-236 to I-250; P-237 to A-251; E-238 to K-252; T-239 to L-253; L-240 to E-254; P-241 to E-255; N-242 to G-256; N-243 to D-257; S-244 to E-258; C-245 to L-259; Y-246 to Q-260; S-247 to L-261; A-248 to A-262; G-249 to I-263; 1-250 to P-264; A-251 to R-265; K-252 to E-266; L-253 to N-267; E-254 to A-268; E-255 to Q-269; G-256 to I-270; D-257 to S-271; E-258 to L-272; L-259 to D-273; Q-260 to G-274; L-261 to D-275; A-262 to V-276; I-263 to T-277; P-264 to F-278; R-265 to F-279; E-266 to G-280; N-267 to A-281; A-268 to L-282; Q-269 to K-283; 1-270 to L-284; and S-271 to L-285 of the sequence listed in Table 2. Additionally, coordinates comprising, or alternatively, consisting of, coordinates of an amino acid sequence at least 80%, 85%, 90%, 92%, 95%, 96%, 97%, 98% or 99% identical to an amino acid sequence described above.

Methods of Assaying Compounds that Interact with Neutrokine-Alpha

It is possible to define ligand interactions with a Neutrokine-alpha protein. Exemplary methods include the following.

(1) Effects of ligand binding upon protein intrinsic fluorescence (e.g., of tryptophan). Binding of either natural ligands or inhibitors may result in enzyme conformational changes which alter the fluorescence of a Neutrokine-alpha protein.

(2) Spectral effects of ligands. Where the ligands themselves are either fluorescent or possess chromophores that overlap with enzyme tryptophan fluorescence, binding can be detected either via changes in the ligand fluorescence properties (e.g., intensity, lifetime, or polarization) or fluorescence resonance energy transfer with enzyme tryptophans.

(3) Thermal analysis of the Neutrokine-alpha:ligand complex. Using calorimetric techniques (e.g., isothermal calorimetry or differential scanning calorimetry), it is possible to detect thermal changes, or shifts in the stability of a Neutrokine-alpha protein which reports and therefore allows the characterization of ligand binding.

(4) Surface plasmon resonance spectroscopy. A BIACORE Surface plasmon resonance analyzer can be used to measure binding of a ligand to a Neutrokine-alpha protein.

Additional methods are known in the art and are disclosed in, for example, WO 98/18921, published May 7, 1998; and WO 00/50597, published Aug. 31, 2000.

Computer-Related Embodiments

Another aspect of the present invention is a computer readable medium comprising a the three-dimensional structure of a Neutrokine-alpha protein or a portion thereof. The X-ray diffraction data, atomic coordinate data, and amino acid sequence data of the present invention can be provided as a manufacture in a variety of media to facilitate use thereof. As used herein, “computer readable medium refers to any medium which can be read and accessed directly by a computer. Such a medium includes, but is not limited to, magnetic storage media, such as floppy discs, hard disc storage medium, and magnetic tape; optical storage media such as optical discs or CD-ROM; electrical storage media such as RAM and ROM; and hybrids of these categories such as magnetic/optical storage media. A skilled artisan can readily appreciate how any of the presently known computer readable media can be used to create a manufacture comprising a computer readable medium having recorded thereon the X-ray diffraction data, atomic coordinate data, or amino acid sequence data of the present invention.

A variety of data storage structures are available to a skilled artisan for creating a computer readable medium having recorded thereon the above-described data. The choice of the data storage structure will generally be based on the means chosen to access the stored information. For example, the data can be represented in a word processing text file, formatted in commercially-available software such as WordPerfect and MICROSOFT Word, or represented in the form of an ASCII file, stored in a database application, such as DB2, Sybase, Oracle, or the like. A skilled artisan can readily adapt any number of data processor structuring formats (e.g., text file or database) in order to obtain a computer readable medium according to the present invention.

By providing on computer readable media having stored thereon the above-described data, a skilled artisan can routinely access the amino acid sequence, atomic coordinate, or X-ray diffraction data to model a nuclear receptor ligand using model building methods known in the art or described herein. Computer algorithms are publicly and commercially available which allow a skilled artisan to access this data provided on a computer readable medium and analyze it for structure determination and rational design of ligands. See, e.g., Biotechnology Software Directory, Mary Ann Liebert Publ., New York (1995).

The present invention further provides systems, particularly computer-based systems, which contain the amino acid sequence data, diffraction data, and/or atomic coordinate data described herein. Such systems are designed to perform structure determinations of nuclear receptors and the rational design of their ligands. Non-limiting examples are microcomputer workstations available from SGI or Sun Microsystems running Unix-based, Windows NT, or IBM OS/2 operating systems.

As used herein, “a computer-based system” refers to the hardware means, software means, and data storage means used to analyze the amino acid sequence data, X-ray diffraction data, and/or atomic coordinate data of the present invention. The minimum hardware means of the computer-based systems of the present invention comprises a central processing unit (CPU), input means, output means, and data storage means. A skilled artisan can readily appreciate which of the currently available computer-based system are suitable for use in the present invention. A monitor is optionally provided to visualize structure data.

As used herein, “data storage means” refers to memory which can store the data of the present invention, or a memory access means which can access manufactures having the data recorded thereon. As used herein, “data-analyzing means” refers to one or more of the above-described or art-known computer algorithms which are capable of analyzing stored amino acid sequence data, X-ray diffraction data, and/or atomic coordinate data and producing a refined model of the three dimensional structure of a neutrokine-alpha protein.

Alternatively, the three dimensional structure of a Neutrokine-alpha protein, or portion thereof, can be stored on a computer readable medium via the “data storage means” described above. After being retrieved, data corresponding to the model can be analyzed by the “data analyzing means.” In such a scenario, the data-analyzing means refers to any of the known computer algorithms (described below), which, based on the model of the neutrokine-alpha protein, indicates the three dimensional structure of compounds capable of binding to the neutrokine-alpha protein. Such candidate ligands, may act as a agonist or antagonist of the neutrokine-alpha protein pathway. Methods for determining whether a compound acts as an agonist or antagonist of a neutrokine-alpha protein are described above.

### EXAMPLES

### Example 1

### Human Neutrokine-Alpha in Crystalline Form

Human Neutrokine-alpha protein in crystalline form was prepared according to the method described in Example 2 or 3. The space group was hexagonal having unit cell dimensions of a=123.58 Å, b=123.58 Å, c=161.23 Å, α=90, β=90, and γ=120 and was later determined to be P6₅. Crystal density measurements using Ficoll gradients indicated (Z=6) six Neutrokine-alpha monomers/asymmetric unit. For more details regarding Ficoll gradients, see Westbrook, E. M. Methods Enzymol. 114:187-96 (1985). The Matthew's coefficient for these crystals was calculated to be 3.58 Å³/Da with solvent content of 65%, using 912 residues.

### Example 2

### Preparing Human Neutrokine-Alpha in Crystalline Form

Isolation of full length Neutrokine-alpha cDNA. The BLAST algorithm was used to search the Human Genome Sciences Inc. expressed sequence tag (EST) database for sequences with homology to the receptor-binding domain of the TNF family. A full length Neutrokine-alpha clone was identified, sequenced and submitted to GenBank (Accession number AF132600). The Neutrokine-alpha open reading frame was PCR amplified utilizing a 5′ primer (5′-CAG ACT GGA TCC GCC ACC ATG GAT GAC TCC ACA GAA AG-3′) annealing at the predicted start codon and a 3′ primer (5′-CAG ACT GGT ACC GTC CTG COT GCA CTA CAT GGC-3′) designed to anneal at the predicted downstream stop codon. The resulting amplicon was tailed with Bam HI and Asp 718 restriction sites and subcloned into a mammalian expression vector. Neutrokine-alpha was also expressed in p-CMV-1 (Sigma Chemicals).

Purification of recombinant human Neutrokine-alpha. Neutrokine-alpha protein was expressed in insect Sf9 cells using a recombinant baculovirus system as described in Moore et al., Science 285:260-263 (1999). Sf9 cell supernatant was treated with 10 mM calcium chloride in slightly alkaline conditions. Neutrokine-alpha was purified through a Poros PI-50 (Applied BioSystem, Framingham, Mass.) column and a Toyopearl Hexyl 650C (TosoHaas, Montgomeryville, Pa.) column. The final purified Neutrokine-alpha protein was diafiltered into a buffer containing 10 mM sodium citrate, 140 mM sodium chloride pH 6.

Crystallization. The sparse matrix approach was used to screen for crystals. See Jancarik, J. & Kim, S. H., “Sparse matrix sampling: a screening method for crystallization of proteins.” J. Appl. Cryst. 24:409-411 (1991) for full details.

Crystal Screen II (Hampton Research, Riverside Calif.), condition #4, 35% (w/v) dioxane in water provided crystals. Using a fresh Hampton kit required the addition of divalent cations (Mg²⁺ or Zn²⁺) to obtain high-resolution crystals. Crystals were grown in hanging drops containing 1 mL of 20 mg/mL hNeutrokine-alpha in 25 mM sodium citrate, 125 mM NaCl, pH 6 and 1 mL of 25% dioxane, 25 mM MgCl₂ suspended over a reservoir of 25% dioxane, 25 mM MgCl₂. Crystals formed overnight.

### Example 3

### Preparing Human Neutrokine-Alpha in Crystalline Form

Isolation of full length Neutrokine-alpha cDNA. The BLAST algorithm was used to search the Human Genome Sciences Inc. expressed sequence tag (EST) database for sequences with homology to the receptor-binding domain of the TNF family. A full length Neutrokine-alpha clone was identified, sequenced and submitted to GenBank (Accession number AF132600). The Neutrokine-alpha open reading frame was PCR amplified utilizing a 5′ primer (5′-CAG ACT GGA TCC GCC ACC ATG GAT GAC TCC ACA GAA AG-3′) annealing at the predicted start codon and a 3′ primer (5′-CAG ACT GGT ACC GTC CTG CGT GCA CTA CAT GGC-3′) designed to anneal at the predicted downstream stop codon. The resulting amplicon was tailed with Bam HI and Asp 718 restriction sites and subcloned into a mammalian expression vector. Neutrokine-alpha was also expressed in p-CMV-1 (Sigma Chemicals).

Purification of recombinant human Neutrokine-alpha. Neutrokine-alpha protein was expressed in insect Sf9 cells using a recombinant baculovirus system as described in Moore et al., Science 285:260-263 (1999). Sf9 cell supernatant was treated with 10 mM calcium chloride in slightly alkaline conditions. Neutrokine-alpha was purified through a Poros PI-50 (Applied BioSystem, Framingham, Mass.) column, Sephacryl S200 size exclusion (Amersham Pharmacia Biotech), a Toyopearl Hexyl 650C (TosoHaas, Montgomeryville, Pa.) column, and a DEAE sepharose column (Amersham Pharmacia Biotech). The final purified Neutrokine-alpha protein was diafiltered into a buffer containing 25 mM sodium citrate, 125 mM sodium chloride pH 6.

Crystallization. The sparse matrix approach was used to screen for crystals. See Jancarik, J. & Kim, S. H., “Sparse matrix sampling: a screening method for crystallization of proteins.” J. Appl. Cryst. 24:409-411 (1991) for full details.

Crystal Screen II (Hampton Research, Riverside Calif.), condition #4, 35% (v/v) dioxane in water provided crystals. Using a fresh Hampton kit required the addition of divalent cations (Mg²⁺ or Zn²⁺) to obtain high-resolution crystals. Crystals were grown in hanging drops containing 1 μL of 20 mg/mL hNeutrokine-alpha in 25 mM sodium citrate, 125 mM NaCl, pH 6 and 1 μL of 25% dioxane, 25 mM MgCl₂ suspended over a reservoir of 25% dioxane, 25 mM MgCl₂. Crystals formed overnight.

Crystals were flash-cooled for data collection by rapid transfer into 25% (v/v) glycerol, 25% (v/v) dioxane and 25 mM MgCl₂, followed by direct replacement into the liquid nitrogen stream.

### Example 4

### Preparing NonHuman Neutrokine-Alpha in Crystalline Form

Mouse Neutrokine-alpha in crystalline form is prepared according to the method as described for human Neutrokine-alpha. Mouse Neutrokine-alpha has the following sequence:
1 mdesaktlpp pclcfcsekg edmkvgydpi tpqkeegawf gicrdgrlla atlllallss
61 sftamslyql aalqadlmnl rmelqsyrgs atpaaagape ltagvklltp aaprphnssr
121 ghrnrrafqg peeteqdvdl sappapclpg crhsqhddng mnlrniiqdc lqliadsdtp
181 tirkgtytfv pwllsfkrgn aleekenkiv vrqtgyffiy sqvlytdpif amghviqrkk
241 vhvfgdelsl vtlfrciqnm pktlpnnscy sagiarleeg deiqlaipre naqisrngdd
301 tffgalkll
(GenBank Accession No.: AAD22475; Schneider et al., J. Exp. Med. 189:1747-1756 (1999)). The protein used comprises residues 131-301.

### Example 5

### Determining the Three-Dimensional Structure of Human Neutrokine-Alpha in Crystalline Form

Data collection and processing. The flash cooling of crystals was performed by a quick-transfer of the crystal into an aqueous solution comprising 25% glycerol, 25% dioxane, and 25 mM MgCl₂. The solution containing the crystal was directly placed it in the liquid N₂ stream. All data were collected from one frozen crystal on the Cornell High Energy Synchrotron Source (CHESS) in Ithaca, N.Y., on the F1 beam line using the Quantum4 detector. The wavelength was 0.942 Å. At a crystal-to-film distance of 190 mm, 40-second exposures of 1° oscillation were collected through 180 degrees of rotation. The crystal was moved, and an additional 70 degrees of data were collected. Intensities were integrated, reduced, and scaled with the Denzo/Scalepack package. The data collection statistics are shown in below.

Molecular Replacement. Starting models were chosen from the homologs: TNF (Protein Data Bank Entry Number: 1TNR), Apo2L/TRAIL (Protein Data Bank Entry Number: 1 DOG) and CD40 Ligand (Protein Data Bank Entry Number: 1ALY) using monomers and/or trimers that have been stripped of side-chains. A model for Amore (Navaza, 2001) was created from Apo2L/TRAIL in which the side-chains of residues identical (based on sequence alignments) between it and Neutrokine-alpha were not removed. This “pruned” model gave the best statistics (see Tables 1A and 1B) and was used to find first one trimer and then the other trimer to complete the asymmetric unit. The TNF-β model had offered the same solution. CNS (Brunger, A. T. et al., ActaCrystallogr. D. 54:905-921 (1998)) was used to calculate the phases and to create a solvent flattened map calculated with 60% solvent content using SIGMAA weighting (Read, R. J. Acta Crystallogr. A 42:140-149 (1986)). This solvent flattened map, with the phases calculated from the model and Amore solution at both 3.5 Å and 2.0 Å, was fully interpretable. All the segments of protein structure that differed between the model and Neutrokine-alpha were apparent in this map, including new loops, disulfide bonds, and density of ligand.

Model building and Refinement. One monomer of 143 amino acids was modeled within 48 hours and then duplicated using the “lsq” module in O (http://www.imsb.au.dk/˜mok/o/) onto the other 5 location in the asymmetric unit. One round of simulated annealing at 2000° C. with maximum likelihood refinement resulted in R of 25.97% and R_{free} of 28.37% at 2 Å resolution. Subsequent cycles of refinement involved addition of metals, ligands (citrate and dioxane), and water molecules to a values of R and R_{free} of 19.2% and 21.2% and final values of R and R_{free} of 18.9% and 20.9%, respectively. The calculations were performed on data with F>1σ(F) using 7486 scattering atoms. Residues 134-141 of each of the monomers were unobserved, and residues 104-106 in each monomer had weak density.

TABLE 1A

| Resolution limits (Å) of data collected | | 30.0-2.0 |
|---|---|---|
| Number of | total | 1,940,104 |
| reflections | unique | 93,234 |
| Completeness | overall | 99.3 |
| (%) | last shell (2.03 Å-2.0 Å) | 86.6 |
| Rsym (%) | overall | 8.3 |
| | last shell (2.03 Å-2.0 Å) | 39.4 |
| Molecular | model | PDB entry 1D0G |
| replacement | resolution range (Å) | 30.0-4.5 |
| | Rfactor for a trimer | 52.4 |
| | correlation for trimer | 12.2 |
| | Rfactor for two trimers | 49.8 |
| | correlation for two trimers | 25.8 |
| Refinement | resolution range (Å) | 25.0-2.0 |
| | number of reflections in working set | 91,331 |
| | number of reflections used for Rfree | 1849 (5%) |
| | Rcryst | 19.2 |
| | Rfree | 21.2 |
| | number of atoms in protein | 6865 |
| | number of ligand atoms | 108 |
| | number of water molecules | 513 |
| Geometry | deviations in bond length (Å) | 0.007 |
| | deviations in bond angles (°) | 1.31 |

TABLE 1B

| Resolution limits (Å) of data collected | | 30.0-2.0 |
|---|---|---|
| Number of | total | 1,940,104 |
| reflections | unique | 93,234 |
| Completeness | overall | 99.3 |
| (%) | last shell (2.03 Å-2.0 Å) | 86.6 |
| Rsym (%) | overall | 8.3 |
| | last shell (2.03 Å-2.0 Å) | 39.4 |
| Molecular | model | PDB entry 1D0G |
| replacement | resolution range (Å) | 30.0-4.5 |
| | Rfactor for a trimer | 52.4 |
| | correlation for trimer | 12.2 |
| | Rfactor for two trimers | 49.8 |
| | correlation for two trimers | 25.8 |
| Refinement | resolution range (Å) | 25.0-2.0 |
| | number of reflections in working set | 91,331 |
| | number of reflections used for Rfree | 1849 (5%) |
| | Rcryst | 18.9 |
| | Rfree | 20.9 |
| | number of atoms in protein | 6858 |
| | number of ligand atoms | 153 |
| | number of water molecules | 462 |
| Geometry | deviations in bond length (Å) | 0.007 |
| | deviations in bond angles (°) | 1.30 |

### Example 6

### Energy Minimization of Neutrokine-Alpha Structure

The coordinates of IKXG were subjected to an energy minimization process. Specifically, missing atoms, such as hydrogen atoms were removed, as well as water molecules. The resulting dimer of trimers was minimized using the Powell minimization algorithm, first without electrostatics for 100 cycles. The coordinates resulting from the Powell minimization were then subjected to a second minimization using electrostatic and van der Waals forces using the Tripos60 algorithm. The resulting total energy of the minimized dimer of trimers was approximately −2639 Kcal/mol. The root mean square deviation between the minimized protein structure and the unminimized protein structure (i.e., 1KXG) was about 0.27 Å, calculated based on alpha-carbon backbone. Table 6 below provides the coordinates a single monomer of the energy-minimized neutrokine-alpha protein.

### Example 7

### Determining the Solvent Accessible Surface of Human Neutrokine-Alpha in Crystalline Form

The coordinates of Table 2 are used to display the structure of hNeutrokine-alpha using a suitable computer program, such as Sybyl 6.5. Oxygen atoms of associated water molecules are deleted. According to accepted and standard protocol, hydrogen atoms and atomic charges are added to the structure. The structure is then minimized using a standard molecular mechanics force field, such as the Tripos force field. The solvent accessible surface is then calculated and displayed using the MOLCAD™ module. The resulting structure and visualization provide a graphical display of the solvent accessible surface of a dimer of trimerized human Neutrokine-alpha. This graphical display can then be further used to identify potential binding sites for molecules and receptors.

### Example 8

### Determining the Molecular Lipophilic Potential Surface of Human Neutrokine-Alpha in Crystalline Form

The coordinates of Table 2 corresponding to one trimer are used to display the structure of hNeutrokine-alpha using a suitable computer program, such as Sybyl 6.5. Oxygen atoms of associated water molecules are deleted. According to accepted and standard protocol, hydrogen atoms and atomic charges are added to the structure. The structure is then minimized using a standard molecular mechanics force field, such as the Tripos force field. The solvent accessible surface is then calculated and displayed using the MOLCAD™ module. The resulting structure and visualization provides the a graphical display of the lipophilic potential surface of a dimer of trimerized human Neutrokine-alpha. This graphical display can then be further used to identify potential binding sites for molecules and receptors. Specifically, an area of low lipophilic potential is identified as potential binding site for a hydrophilic moiety of a compound.

### Example 9

### Determining the Structure of Modified Human Neutrokine-Alpha by Molecular Modeling

The coordinates of hNeutrokine-alpha listed in Table 2 are entered into a computer system using a standard molecular modeling software program such as SYBYL 6.5, according to the known procedures. Hydrogen atoms are added to the coordinates. Charges are assigned to each of the atoms according to known routines. The structure of human Neutrokine-alpha is then further minimized using a molecular mechanics force field such as AMBER. Phenylalanine-220, located in the D-E loop is changed to an alanine residue (i.e., F220-->A220 mutation). The local region of the structure comprising the atoms of the D-E loop are then subject to molecular mechanics minimization again. The resulting structure provides the three dimensional structure of a modified hNeutrokine-alpha protein, specifically of F220A hNeutrokine-alpha. The solvent accessible surface is optionally calculated and displayed to provide an additional representation of the three dimensional structure of a modified human Neutrokine-alpha protein.

### Example 10

### Determining the Structure of Mouse Neutrokine-Alpha by Homology Modeling

A model of the mouse Neutrokine-alpha is constructed using Quanta version 4.1 [Molecular Simulations Inc, Burlington, Mass.]. Specifically, the MODELER module within Quanta is used. Alternatively, the MODELER module of INSIGHT II may be used. The coordinates of hNeutrokine-alpha, as listed in Table 2, are used as the template structure. The sequence of mouse Neutrokine-alpha is provided in Example 3.

Residues 131-301 of mouse Neutrokine-alpha are used to construct the three-dimensional model of mouse Neutrokine-alpha. According to standard and well-known methods, hydrogen atoms and atomic charges are added to the resulting three-dimensional model of mouse Neutrokine-alpha. A representation of the solvent accessible surface of the mouse Neutrokine-alpha is optionally added to and displayed on the three-dimensional model of mouse Neutrokine-alpha. A representation of the lipophilic potential is optionally added to and displayed on the three-dimensional model of mouse Neutrokine-alpha.

### Example 11

### Designing a Compound that Binds to hNeutrokine-Alpha

The three-dimensional structure of hNeutrokine-alpha as a trimer is displayed on a suitable computer system. Specifically, hNeutrokine-alpha as a trimer corresponds to atoms 1-3436 of Table 2. In particular, all amino acid residues within 10 Å of the groove defined by one side of loop DE with some residues of loops aa′ and GH, and on the other side are found loops EF, Aa, and a′A″. Hydrogen atoms are added, and the structure further minimized using AMBER forcefield. A peptide of the sequence EYFDSLLHACIPCQLRCSSNTPPLTC is constructed and minimized. The minimized peptide is then docked manually to the groove on the hNeutrokine-alpha structure. Alternatively, the program AUTODOCK is used to dock the peptide to the hNeutrokine-alpha trimer. Based on the binding mode analysis, portions of the peptide are changed to enhance binding to hNeutrokine-alpha. A compound designed according to this method is useful as an antagonist of hNeutrokine-alpha binding to and activating one or all of the receptors to which hNeutrokine-alpha binds.

### Example 12

### Designing a Compound that is Similar to a Portion of Neutrokine-Alpha

A cyclic peptide corresponding to the loop between D and E is prepared as a compound that binds to Neutrokine-alpha. The sequence is: CRKKVHVFGDELSC. The two terminal cysteines are used to form an intramolecular disulfide bond. The structure of the cyclic peptide is first modeled using standard molecular modeling techniques. The model of the cyclic peptide is compared to the DE loop of the three-dimensional structure of hNeutrokine-alpha. Sufficient structural and chemical similarity is observed to prepare the cyclic peptide. The cyclic peptide is synthesized on an Advanced ChemTech 440 Automated Solid Phase Organic Synthesizer (Advanced ChemTech, Inc., Louisville, Ky.) using standard Fmoc chemistry (e.g., see Jameson et al., Nature 368:744-746 (1994)). The linear peptide is then cyclized under standard oxidizing conditions. The peptide is monitored for purity using reverse-phase high-performance liquid chromatography. The peptide is then preparatively fractioned to greater than 99% purity on an HPLC and its mass verified by mass spectrometry. The cyclic peptide is then assayed for activity.

Other cyclic peptides may be prepared in which certain residues are modified. For example, the phenylalanine of the above cyclic peptide may be mutated to a tyrosine.

### Example 13

### Computer System Comprising

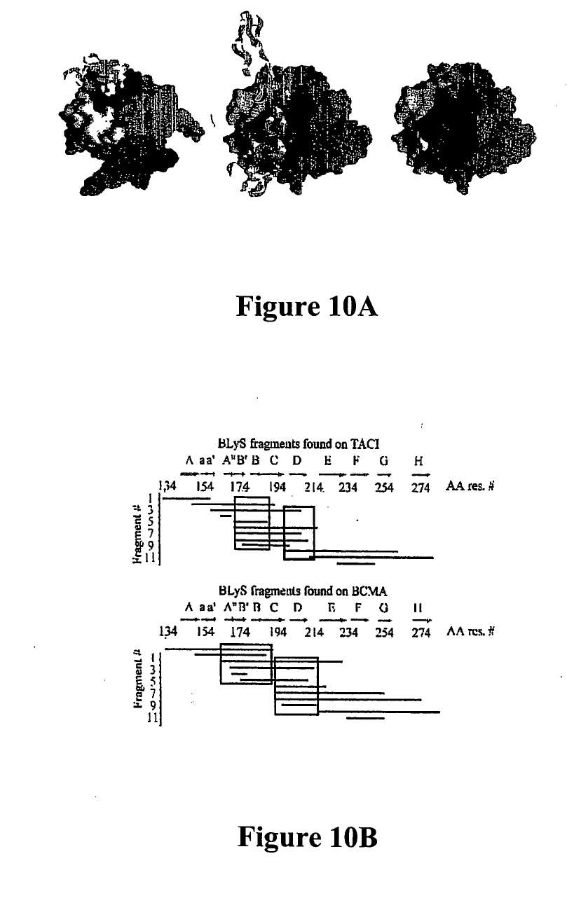

One application of the present invention is provided in FIG. 10, which provides a block diagram of a computer system 102 that can be used to implement the present invention. The computer system 102 includes a processor 106 connected to a bus 104. Also connected to the bus 104 are a main memory 108 (preferably implemented as random access memory, RAM) and a variety of secondary storage memory 110, such as a hard drive 112, a removable storage medium 114, and a monitor 120. The removable medium storage device 114 may represent, for example, a floppy disk drive, a CD-ROM drive, a magnetic tape drive, or a ZIP™ disk. A removable storage medium 116 (such as a floppy disk, a compact disk, a magnetic tape, or a ZIP™ disk) containing control logic and/or data recorded therein may be inserted into the removable medium storage medium 114. The computer system 102 includes appropriate software for reading the control logic and/or the data from the removable medium storage device 114 once inserted in the removable medium storage device 114.

Amino acid sequence data, X-ray diffraction data, and/or atomic coordinate data of the present invention or data corresponding to a model of a nuclear receptor may be stored in a well known manner in the main memory 108, any of the secondary storage devices 110, and/or a removable storage device 116. Software for accessing and processing the data resides in main memory 108 during execution. The monitor 120 is optionally used for visualization.

### Example 14

### SELDI Experiment

SELDI mass spectrometry and data analysis. A surface-enhanced laser desorption-ionization (SELDI) approach was used to identify regions involved in neutrokine-alpha protein binding to receptors TACI and BCMA. Recombinant receptor proteins, tagged with an immunoglobulin Fc domain, were expressed in CHO cells (TACI) or baculovirus-infected insect cells (BCMA) and tested for binding activity by BIACORE and cell-based assays. The receptors were then covalently bound to PS2 ProteinChip™ Arrays (Ciphergen Biosystems) and subsequently incubated with recombinant neutrokine-alpha. After removal of unbound material, the complexes were digested with a high concentration of trypsin. Unretained digest fragments were removed by a stringency wash. The energy-absorbing molecule, α-hydroxy-cinnaminic acid (CHCA) in 10% (v/v) formic acid and 10% (v/v) ethanol, was added, and chips were analyzed on a Ciphergen PBS 2, as well as a PE Sciex Qstar with protein chip interface. PS2 data with four-point external calibration achieve a mass accuracy of ˜50-100 p.p.m., and QStar data have 5 p.p.m. accuracy. Fragment matches and distributions were analyzed using PAWS (Protein Analysis Worksheet, Proteometrics).

LENGTHY TABLE REFERENCED HERE
US20070026500A1-20070201-T00001
Please refer to the end of the specification for access instructions.

Table 2 provides the atomic coordinates of the three-dimensional structure of hNeurokine-alpha. Specifically, the above coordinates comprise the residues 141-285 of hNeutrokine-alpha in crystal form. Moreover, the entire set of coordinates listed in Table 2 comprise the hNeutrokine-alpha protein in crystalline form as a dimer of trimers. The coordinates listed in Table 2 further comprise The following provides a description of the columns listed in above Table 2. The coordinates listed in Table 2 are used in a standard PDB file format, as described in http://www.rcsb.org/pdb/docs/format/pdbguide2.2/guide2.2_frame.html. The set of atomic coordinates of Table 2, or equivalent thereof, has been deposited into and is available from the Protein Data Bank: I.D. No.: 1KXG. (H. M. Berman, et al., The Protein Data Bank. Nucleic Acids Research, 28 pp. 235-242 (2000)).

Column 1 indicates the atom number of each atom listed in coordinates of human Neutrokine-alpha. The atoms are numbered numerically from 1-7521.

Column 2 indicates the atom name, according to standard nomenclature for Protein Data Bank files. The following Table 3 provides the standard information regarding the atom names in column 2 of Table 2.

TABLE 3
Atom types.

| Atom Name | Type of Atom |
|---|---|
| C | Carbonyl carbon of peptide bond |
| N | Nitrogen of peptide bond |
| O | Oxygen of peptide bond |
| CA | Alpha carbon |
| CB | Beta carbon |
| CG | Gamma carbon |
| CD | Delta carbon |
| CE | Epsilon carbon |
| NA | Alpha nitrogen |
| NB | Beta nitrogen |
| NG | Gamma nitrogen |
| ND | Delta nitrogen |
| DE | Epsilon nitrogen |
| OA | Alpha oxygen |
| OB | Beta oxygen |
| OD | Delta oxygen |
| OE | Epsilon oxygen |
| OH | Tyrosine hydroxyl oxygen |
| OH2 | Oxygen of water molecule |
| OXT | Terminal oxygen of peptide chain |
| MG | Magnesium ion |

In addition, when the residue type is CIT (see column 3, infra), the atom names C1, C2, C3, C4, C5, C6, O1, O2, O3, O4, O5, O6, and O7 refer to non-hydrogen atoms that comprise a citrate molecule. When the residue type is DIO (see column 3, infra), the atom names C1, C2, C1′, C2′, O1, and O1′ refer to the non-hydrogen atoms comprise that a dioxane molecule.

Column 3 indicates the residue type according to standard nomenclature for Protein Data Bank files. Additionally, MG indicates a magnesium ion; CIT indicates a citrate molecule; TIP indicates a water molecule; DIO indicates a dioxane molecule.

Column 4 indicates the fragment identification. The letter A indicates protein subunit A. The letter B indicates protein subunit B. The letter C indicates protein subunit C. The letter D indicates protein subunit D. The letter E indicates protein subunit E. The letter F indicates protein subunit F. One trimer of hNeutrokine-alpha comprises subunits A, B, and C; a second trimer comprises subunits D, E, and F. As described above, each trimer comprises three monomers, or subunits. As is apparent, each monomer of hNeutrokine-alpha is represented by one of subunits A-F. The letters G and H indicate Magnesium or Water atoms. The letter I indicates citrate atoms. The letter K indicates dioxane atoms. The letter U, V, W, X, Y and Z indicate water atoms.

Column 5 indicates the residue number of a particular atom. For atoms part of the protein, the residue number is number of the amino acid residue to which the atom belongs, wherein the number of the amino acid residue is numbered according to standard numbering of Neutrokine-alpha peptide numbering.

Column 6 indicates the x-coordinate in three dimensional space. Column 7 indicates the y-coordinate in three dimensional space. Column 8 indicates the z-coordinate in three dimensional space. The units of each coordinate is angstroms.

Column 9 indicates the occupancy. For all atoms in Table 1, the occupancy value is 1. Column 10 specifies the temperature factor.

TABLE 4

Full Length Amino Acid Sequence of Human
Neutrokine-alpha

| | |
|---|---|
| 1 | mddstereqs rltsclkkre emklkecvsi lprkespsvr |
| | sskdgkllaa tlllallscc |
| 61 | ltvvsfyqva alqgdlaslr aelqghhaek lpagagapka |
| | gleeapavta glkifeppap |
| 121 | gegnssqnsr nkravqgpee tvtqdclqli adsetptiqk |
| | gsytfvpwll sfkrgsalee |
| 181 | kenkilvket gyffiygqvl ytdktyamgh liqrkkvhvf |
| | gdelslvtlf rciqnmpetl |
| 241 | pnnscysagi akleegdelq laiprenaqi sldgdvtffg |
| | alkll |

TABLE 5

Amino Acid Sequence of Soluble Human
Neutrokine-alpha

| | |
|---|---|
| 141 | tvtqdclqli adsetptiqk gsytfvpwll sfkrgsalee |
| 181 | kenkilvket gyffiygqvl ytdktyamgh liqrkkvhvf |
| | gdelslvtlf rciqnmpetl |
| 241 | pnnscysagi akleegdelq laiprenaqi sldgdvtffg |
| | alkll |

TABLE 6
Energy-minimized Structure of Neutrokine-alpha monomer.

| | | | | | | | | | |
|---|---|---|---|---|---|---|---|---|---|
| 1 | N | VAL | E | 142 | 2.070 | −28.495 | −18.257 | 1.00 | 0.00 |
| 2 | CA | VAL | E | 142 | 0.713 | −28.363 | −18.753 | 1.00 | 0.00 |
| 3 | C | VAL | E | 142 | 0.452 | −26.957 | −19.217 | 1.00 | 0.00 |
| 4 | O | VAL | E | 142 | 1.178 | −26.042 | −18.861 | 1.00 | 0.00 |
| 5 | CB | VAL | E | 142 | −0.298 | −28.771 | −17.660 | 1.00 | 0.00 |
| 6 | CG1 | VAL | E | 142 | −1.739 | −28.727 | −18.205 | 1.00 | 0.00 |
| 7 | CG2 | VAL | E | 142 | 0.008 | −30.197 | −17.163 | 1.00 | 0.00 |
| 8 | N | THR | E | 143 | −0.608 | −26.791 | −20.031 | 1.00 | 0.00 |
| 9 | CA | THR | E | 143 | −0.942 | −25.454 | −20.486 | 1.00 | 0.00 |
| 10 | C | THR | E | 143 | −2.427 | −25.239 | −20.400 | 1.00 | 0.00 |
| 11 | O | THR | E | 143 | −3.163 | −26.143 | −20.041 | 1.00 | 0.00 |
| 12 | CB | THR | E | 143 | −0.451 | −25.216 | −21.928 | 1.00 | 0.00 |
| 13 | OG1 | THR | E | 143 | −1.007 | −26.198 | −22.810 | 1.00 | 0.00 |
| 14 | CG2 | THR | E | 143 | 1.085 | −25.286 | −21.977 | 1.00 | 0.00 |
| 15 | N | GLN | E | 144 | −2.863 | −24.011 | −20.734 | 1.00 | 0.00 |
| 16 | CA | GLN | E | 144 | −4.279 | −23.714 | −20.640 | 1.00 | 0.00 |
| 17 | C | GLN | E | 144 | −4.871 | −23.691 | −22.017 | 1.00 | 0.00 |
| 18 | O | GLN | E | 144 | −4.582 | −22.791 | −22.790 | 1.00 | 0.00 |
| 19 | CB | GLN | E | 144 | −4.460 | −22.346 | −19.956 | 1.00 | 0.00 |
| 20 | CG | GLN | E | 144 | −3.696 | −22.334 | −18.617 | 1.00 | 0.00 |
| 21 | CD | GLN | E | 144 | −3.859 | −20.993 | −17.959 | 1.00 | 0.00 |
| 22 | OE1 | GLN | E | 144 | −2.904 | −20.238 | −17.881 | 1.00 | 0.00 |
| 23 | NE2 | GLN | E | 144 | −5.079 | −20.693 | −17.475 | 1.00 | 0.00 |
| 24 | N | ASP | E | 145 | −5.714 | −24.696 | −22.320 | 1.00 | 0.00 |
| 25 | CA | ASP | E | 145 | −6.343 | −24.711 | −23.628 | 1.00 | 0.00 |
| 26 | C | ASP | E | 145 | −7.271 | −23.540 | −23.772 | 1.00 | 0.00 |
| 27 | O | ASP | E | 145 | −7.804 | −23.060 | −22.785 | 1.00 | 0.00 |
| 28 | CB | ASP | E | 145 | −7.126 | −26.019 | −23.854 | 1.00 | 0.00 |
| 29 | CG | ASP | E | 145 | −6.221 | −27.217 | −23.937 | 1.00 | 0.00 |
| 30 | OD1 | ASP | E | 145 | −4.975 | −27.039 | −24.011 | 1.00 | 0.00 |
| 31 | OD2 | ASP | E | 145 | −6.761 | −28.355 | −23.922 | 1.00 | 0.00 |
| 32 | N | CYS | E | 146 | −7.452 | −23.073 | −25.021 | 1.00 | 0.00 |
| 33 | CA | CYS | E | 146 | −8.322 | −21.929 | −25.217 | 1.00 | 0.00 |
| 34 | C | CYS | E | 146 | −8.802 | −21.859 | −26.639 | 1.00 | 0.00 |
| 35 | O | CYS | E | 146 | −8.309 | −22.575 | −27.496 | 1.00 | 0.00 |
| 36 | CB | CYS | E | 146 | −7.623 | −20.619 | −24.800 | 1.00 | 0.00 |
| 37 | SG | CYS | E | 146 | −6.083 | −20.415 | −25.749 | 1.00 | 0.00 |
| 38 | N | LEU | E | 147 | −9.784 | −20.971 | −26.876 | 1.00 | 0.00 |
| 39 | CA | LEU | E | 147 | −10.280 | −20.820 | −28.228 | 1.00 | 0.00 |
| 40 | C | LEU | E | 147 | −10.839 | −19.436 | −28.394 | 1.00 | 0.00 |
| 41 | O | LEU | E | 147 | −11.443 | −18.901 | −27.477 | 1.00 | 0.00 |
| 42 | CB | LEU | E | 147 | −11.341 | −21.903 | −28.516 | 1.00 | 0.00 |
| 43 | CG | LEU | E | 147 | −11.620 | −22.025 | −30.029 | 1.00 | 0.00 |
| 44 | CD1 | LEU | E | 147 | −12.272 | −23.389 | −30.325 | 1.00 | 0.00 |
| 45 | CD2 | LEU | E | 147 | −12.552 | −20.887 | −30.494 | 1.00 | 0.00 |
| 46 | N | GLN | E | 148 | −10.620 | −18.852 | −29.586 | 1.00 | 0.00 |
| 47 | CA | GLN | E | 148 | −11.133 | −17.517 | −29.809 | 1.00 | 0.00 |
| 48 | C | GLN | E | 148 | −11.894 | −17.461 | −31.102 | 1.00 | 0.00 |
| 49 | O | GLN | E | 148 | −11.538 | −18.134 | −32.057 | 1.00 | 0.00 |
| 50 | CB | GLN | E | 148 | −9.962 | −16.519 | −29.804 | 1.00 | 0.00 |
| 51 | CG | GLN | E | 148 | −10.501 | −15.078 | −29.736 | 1.00 | 0.00 |
| 52 | CD | GLN | E | 148 | −9.421 | −14.159 | −29.229 | 1.00 | 0.00 |
| 53 | OE1 | GLN | E | 148 | −8.925 | −13.338 | −29.983 | 1.00 | 0.00 |
| 54 | NE2 | GLN | E | 148 | −9.056 | −14.287 | −27.940 | 1.00 | 0.00 |
| 55 | N | LEU | E | 149 | −12.963 | −16.644 | −31.104 | 1.00 | 0.00 |
| 56 | CA | LEU | E | 149 | −13.738 | −16.500 | −32.320 | 1.00 | 0.00 |
| 57 | C | LEU | E | 149 | −13.939 | −15.044 | −32.620 | 1.00 | 0.00 |
| 58 | O | LEU | E | 149 | −13.784 | −14.203 | −31.749 | 1.00 | 0.00 |
| 59 | CB | LEU | E | 149 | −15.101 | −17.208 | −32.210 | 1.00 | 0.00 |
| 60 | CG | LEU | E | 149 | −14.908 | −18.731 | −32.070 | 1.00 | 0.00 |
| 61 | CD1 | LEU | E | 149 | −16.288 | −19.393 | −31.896 | 1.00 | 0.00 |
| 62 | CD2 | LEU | E | 149 | −14.201 | −19.314 | −33.310 | 1.00 | 0.00 |
| 63 | N | ILE | E | 150 | −14.282 | −14.756 | −33.888 | 1.00 | 0.00 |
| 64 | CA | ILE | E | 150 | −14.435 | −13.365 | −34.277 | 1.00 | 0.00 |
| 65 | C | ILE | E | 150 | −15.553 | −13.210 | −35.267 | 1.00 | 0.00 |
| 66 | O | ILE | E | 150 | −15.952 | −14.173 | −35.903 | 1.00 | 0.00 |
| 67 | CB | ILE | E | 150 | −13.117 | −12.803 | −34.847 | 1.00 | 0.00 |
| 68 | CG1 | ILE | E | 150 | −12.577 | −13.747 | −35.941 | 1.00 | 0.00 |
| 69 | CG2 | ILE | E | 150 | −12.081 | −12.657 | −33.715 | 1.00 | 0.00 |
| 70 | CD1 | ILE | E | 150 | −11.330 | −13.131 | −36.602 | 1.00 | 0.00 |
| 71 | N | ALA | E | 151 | −16.071 | −11.971 | −35.376 | 1.00 | 0.00 |
| 72 | CA | ALA | E | 151 | −17.203 | −11.764 | −36.261 | 1.00 | 0.00 |
| 73 | C | ALA | E | 151 | −16.833 | −11.979 | −37.701 | 1.00 | 0.00 |
| 74 | O | ALA | E | 151 | −15.767 | −11.567 | −38.130 | 1.00 | 0.00 |
| 75 | CB | ALA | E | 151 | −17.805 | −10.361 | −36.066 | 1.00 | 0.00 |
| 76 | N | ASP | E | 152 | −17.739 | −12.647 | −38.439 | 1.00 | 0.00 |
| 77 | CA | ASP | E | 152 | −17.485 | −12.868 | −39.850 | 1.00 | 0.00 |
| 78 | C | ASP | E | 152 | −18.037 | −11.685 | −40.591 | 1.00 | 0.00 |
| 79 | O | ASP | E | 152 | −19.234 | −11.613 | −40.822 | 1.00 | 0.00 |
| 80 | CB | ASP | E | 152 | −18.189 | −14.160 | −40.314 | 1.00 | 0.00 |
| 81 | CG | ASP | E | 152 | −17.671 | −14.592 | −41.657 | 1.00 | 0.00 |
| 82 | OD1 | ASP | E | 152 | −17.517 | −13.720 | −42.555 | 1.00 | 0.00 |
| 83 | OD2 | ASP | E | 152 | −17.408 | −15.813 | −41.821 | 1.00 | 0.00 |
| 84 | N | SER | E | 153 | −17.136 | −10.757 | −40.966 | 1.00 | 0.00 |
| 85 | CA | SER | E | 153 | −17.578 | −9.568 | −41.675 | 1.00 | 0.00 |
| 86 | C | SER | E | 153 | −18.321 | −9.941 | −42.929 | 1.00 | 0.00 |
| 87 | O | SER | E | 153 | −19.424 | −9.460 | −43.139 | 1.00 | 0.00 |
| 88 | CB | SER | E | 153 | −16.342 | −8.714 | −42.024 | 1.00 | 0.00 |
| 89 | OG | SER | E | 153 | −15.393 | −9.479 | −42.776 | 1.00 | 0.00 |
| 90 | N | GLU | E | 154 | −17.710 | −10.812 | −43.757 | 1.00 | 0.00 |
| 91 | CA | GLU | E | 154 | −18.376 | −11.173 | −44.996 | 1.00 | 0.00 |
| 92 | C | GLU | E | 154 | −19.366 | −12.294 | −44.810 | 1.00 | 0.00 |
| 93 | O | GLU | E | 154 | −19.411 | −13.215 | −45.612 | 1.00 | 0.00 |
| 94 | CB | GLU | E | 154 | −17.361 | −11.460 | −46.120 | 1.00 | 0.00 |
| 95 | CG | GLU | E | 154 | −16.407 | −12.594 | −45.698 | 1.00 | 0.00 |
| 96 | CD | GLU | E | 154 | −15.170 | −12.536 | −46.546 | 1.00 | 0.00 |
| 97 | OE1 | GLU | E | 154 | −15.231 | −13.004 | −47.714 | 1.00 | 0.00 |
| 98 | OE2 | GLU | E | 154 | −14.132 | −12.033 | −46.040 | 1.00 | 0.00 |
| 99 | N | THR | E | 155 | −20.179 | −12.201 | −43.739 | 1.00 | 0.00 |
| 100 | CA | THR | E | 155 | −21.214 | −13.201 | −43.539 | 1.00 | 0.00 |
| 101 | C | THR | E | 155 | −22.367 | −12.582 | −42.799 | 1.00 | 0.00 |
| 102 | O | THR | E | 155 | −22.134 | −11.908 | −41.809 | 1.00 | 0.00 |
| 103 | CB | THR | E | 155 | −20.684 | −14.448 | −42.799 | 1.00 | 0.00 |
| 104 | OG1 | THR | E | 155 | −19.753 | −15.138 | −43.638 | 1.00 | 0.00 |
| 105 | CG2 | THR | E | 155 | −21.843 | −15.415 | −42.497 | 1.00 | 0.00 |
| 106 | N | PRO | E | 156 | −23.615 | −12.798 | −43.270 | 1.00 | 0.00 |
| 107 | CA | PRO | E | 156 | −24.759 | −12.233 | −42.586 | 1.00 | 0.00 |
| 108 | C | PRO | E | 156 | −25.000 | −12.924 | −41.274 | 1.00 | 0.00 |
| 109 | O | PRO | E | 156 | −24.432 | −13.975 | −41.023 | 1.00 | 0.00 |
| 110 | CB | PRO | E | 156 | −25.913 | −12.557 | −43.556 | 1.00 | 0.00 |
| 111 | CG | PRO | E | 156 | −25.356 | −13.499 | −44.648 | 1.00 | 0.00 |
| 112 | CD | PRO | E | 156 | −23.829 | −13.593 | −44.462 | 1.00 | 0.00 |
| 113 | N | THR | E | 157 | −25.849 | −12.310 | −40.426 | 1.00 | 0.00 |
| 114 | CA | THR | E | 157 | −26.111 | −12.921 | −39.133 | 1.00 | 0.00 |
| 115 | C | THR | E | 157 | −27.070 | −14.070 | −39.258 | 1.00 | 0.00 |
| 116 | O | THR | E | 157 | −27.729 | −14.215 | −40.275 | 1.00 | 0.00 |
| 117 | CB | TER | E | 157 | −26.648 | −11.882 | −38.128 | 1.00 | 0.00 |
| 118 | OG1 | THR | E | 157 | −27.823 | −11.249 | −38.647 | 1.00 | 0.00 |
| 119 | CG2 | THR | E | 157 | −25.566 | −10.826 | −37.847 | 1.00 | 0.00 |
| 120 | N | ILE | E | 158 | −27.131 | −14.895 | −38.197 | 1.00 | 0.00 |
| 121 | CA | ILE | E | 158 | −28.007 | −16.051 | −38.252 | 1.00 | 0.00 |
| 122 | C | ILE | E | 158 | −29.269 | −15.770 | −37.487 | 1.00 | 0.00 |
| 123 | O | ILE | E | 158 | −29.247 | −15.686 | −36.269 | 1.00 | 0.00 |
| 124 | CB | ILE | E | 158 | −27.269 | −17.268 | −37.658 | 1.00 | 0.00 |
| 125 | CG1 | ILE | E | 158 | −25.948 | −17.498 | −38.421 | 1.00 | 0.00 |
| 126 | CG2 | ILE | E | 158 | −28.164 | −18.516 | −37.772 | 1.00 | 0.00 |
| 127 | CD1 | ILE | E | 158 | −25.112 | −18.581 | −37.714 | 1.00 | 0.00 |
| 128 | N | GLN | E | 159 | −30.386 | −15.635 | −38.223 | 1.00 | 0.00 |
| 129 | CA | GLN | E | 159 | −31.645 | −15.429 | −37.536 | 1.00 | 0.00 |
| 130 | C | GLN | E | 159 | −32.208 | −16.756 | −37.113 | 1.00 | 0.00 |
| 131 | O | GLN | E | 159 | −32.444 | −17.611 | −37.951 | 1.00 | 0.00 |
| 132 | CB | GLN | E | 159 | −32.638 | −14.679 | −38.446 | 1.00 | 0.00 |
| 133 | CG | GLN | E | 159 | −32.096 | −13.281 | −38.806 | 1.00 | 0.00 |
| 134 | CD | GLN | E | 159 | −31.751 | −12.520 | −37.556 | 1.00 | 0.00 |
| 135 | OE1 | GLN | E | 159 | −32.601 | −12.328 | −36.702 | 1.00 | 0.00 |
| 136 | NE2 | GLN | E | 159 | −30.483 | −12.088 | −37.443 | 1.00 | 0.00 |
| 137 | N | LYS | E | 160 | −32.422 | −16.924 | −35.795 | 1.00 | 0.00 |
| 138 | CA | LYS | E | 160 | −32.993 | −18.174 | −35.332 | 1.00 | 0.00 |
| 139 | C | LYS | E | 160 | −33.609 | −18.004 | −33.971 | 1.00 | 0.00 |
| 140 | O | LYS | E | 160 | −32.975 | −17.449 | −33.088 | 1.00 | 0.00 |
| 141 | CB | LYS | E | 160 | −31.914 | −19.272 | −35.302 | 1.00 | 0.00 |
| 142 | CG | LYS | E | 160 | −32.575 | −20.628 | −34.987 | 1.00 | 0.00 |
| 143 | CD | LYS | E | 160 | −31.526 | −21.755 | −34.994 | 1.00 | 0.00 |
| 144 | CE | LYS | E | 160 | −31.121 | −22.097 | −36.440 | 1.00 | 0.00 |
| 145 | NZ | LYS | E | 160 | −30.340 | −23.344 | −36.460 | 1.00 | 0.00 |
| 146 | N | GLY | E | 161 | −34.860 | −18.492 | −33.836 | 1.00 | 0.00 |
| 147 | CA | GLY | E | 161 | −35.564 | −18.372 | −32.571 | 1.00 | 0.00 |
| 148 | C | GLY | E | 161 | −35.782 | −16.933 | −32.199 | 1.00 | 0.00 |
| 149 | O | GLY | E | 161 | −35.798 | −16.614 | −31.021 | 1.00 | 0.00 |
| 150 | N | SER | E | 162 | −35.938 | −16.064 | −33.219 | 1.00 | 0.00 |
| 151 | CA | SER | E | 162 | −36.067 | −14.642 | −32.948 | 1.00 | 0.00 |
| 152 | C | SER | E | 162 | −34.797 | −14.081 | −32.363 | 1.00 | 0.00 |
| 153 | O | SER | E | 162 | −34.833 | −13.113 | −31.620 | 1.00 | 0.00 |
| 154 | CB | SER | E | 162 | −37.306 | −14.323 | −32.088 | 1.00 | 0.00 |
| 155 | OG | SER | E | 162 | −38.486 | −14.837 | −32.713 | 1.00 | 0.00 |
| 156 | N | TYR | E | 163 | −33.659 | −14.715 | −32.710 | 1.00 | 0.00 |
| 157 | CA | TYR | E | 163 | −32.388 | −14.210 | −32.227 | 1.00 | 0.00 |
| 158 | C | TYR | E | 163 | −31.498 | −13.898 | −33.393 | 1.00 | 0.00 |
| 159 | O | TYR | E | 163 | −31.669 | −14.443 | −34.473 | 1.00 | 0.00 |
| 160 | CB | TYR | E | 163 | −31.660 | −15.260 | −31.368 | 1.00 | 0.00 |
| 161 | CG | TYR | E | 163 | −32.306 | −15.392 | −29.995 | 1.00 | 0.00 |
| 162 | CD1 | TYR | E | 163 | −33.255 | −16.390 | −29.761 | 1.00 | 0.00 |
| 163 | CD2 | TYR | E | 163 | −31.939 | −14.518 | −28.969 | 1.00 | 0.00 |
| 164 | CE1 | TYR | E | 163 | −33.830 | −16.520 | −28.494 | 1.00 | 0.00 |
| 165 | CE2 | TYR | E | 163 | −32.512 | −14.653 | −27.701 | 1.00 | 0.00 |
| 166 | CZ | TYR | E | 163 | −33.457 | −15.653 | −27.463 | 1.00 | 0.00 |
| 167 | OH | TYR | E | 163 | −34.026 | −15.787 | −26.201 | 1.00 | 0.00 |
| 168 | N | THR | E | 164 | −30.527 | −13.002 | −33.145 | 1.00 | 0.00 |
| 169 | CA | THR | E | 164 | −29.539 | −12.747 | −34.173 | 1.00 | 0.00 |
| 170 | C | THR | E | 164 | −28.247 | −13.363 | −33.719 | 1.00 | 0.00 |
| 171 | O | THR | E | 164 | −27.796 | −13.098 | −32.615 | 1.00 | 0.00 |
| 172 | CB | THR | E | 164 | −29.396 | −11.233 | −34.417 | 1.00 | 0.00 |
| 173 | OG1 | THR | E | 164 | −30.651 | −10.713 | −34.871 | 1.00 | 0.00 |
| 174 | CG2 | THR | E | 164 | −28.328 | −10.977 | −35.495 | 1.00 | 0.00 |
| 175 | N | PHE | E | 165 | −27.658 | −14.207 | −34.585 | 1.00 | 0.00 |
| 176 | CA | PHE | E | 165 | −26.421 | −14.850 | −34.184 | 1.00 | 0.00 |
| 177 | C | PHE | E | 165 | −25.269 | −14.344 | −35.001 | 1.00 | 0.00 |
| 178 | O | PHE | E | 165 | −25.424 | −14.054 | −36.177 | 1.00 | 0.00 |
| 179 | CB | PHE | E | 165 | −26.546 | −16.381 | −34.280 | 1.00 | 0.00 |
| 180 | CG | PHE | E | 165 | −27.538 | −16.870 | −33.229 | 1.00 | 0.00 |
| 181 | CD1 | PHE | E | 165 | −28.904 | −16.936 | −33.518 | 1.00 | 0.00 |
| 182 | CD2 | PHE | E | 165 | −27.073 | −17.254 | −31.968 | 1.00 | 0.00 |
| 183 | CE1 | PHE | E | 165 | −29.804 | −17.382 | −32.547 | 1.00 | 0.00 |
| 184 | CE2 | PHE | E | 165 | −27.971 | −17.703 | −30.996 | 1.00 | 0.00 |
| 185 | CZ | PHE | E | 165 | −29.336 | −17.766 | −31.287 | 1.00 | 0.00 |
| 186 | N | VAL | E | 166 | −24.097 | −14.226 | −34.349 | 1.00 | 0.00 |
| 187 | CA | VAL | E | 166 | −22.950 | −13.700 | −35.066 | 1.00 | 0.00 |
| 188 | C | VAL | E | 166 | −22.260 | −14.798 | −35.824 | 1.00 | 0.00 |
| 189 | O | VAL | E | 166 | −21.907 | −15.800 | −35.223 | 1.00 | 0.00 |
| 190 | CB | VAL | E | 166 | −21.966 | −13.017 | −34.093 | 1.00 | 0.00 |
| 191 | CG1 | VAL | E | 166 | −20.731 | −12.496 | −34.853 | 1.00 | 0.00 |
| 192 | CG2 | VAL | E | 166 | −22.668 | −11.846 | −33.377 | 1.00 | 0.00 |
| 193 | N | PRO | E | 167 | −22.047 | −14.617 | −37.145 | 1.00 | 0.00 |
| 194 | CA | PRO | E | 167 | −21.292 | −15.600 | −37.893 | 1.00 | 0.00 |
| 195 | C | PRO | E | 167 | −19.890 | −15.580 | −37.356 | 1.00 | 0.00 |
| 196 | O | PRO | E | 167 | −19.190 | −14.592 | −37.508 | 1.00 | 0.00 |
| 197 | CB | PRO | E | 167 | −21.329 | −15.017 | −39.320 | 1.00 | 0.00 |
| 198 | CG | PRO | E | 167 | −21.962 | −13.610 | −39.240 | 1.00 | 0.00 |
| 199 | CD | PRO | E | 167 | −22.518 | −13.423 | −37.816 | 1.00 | 0.00 |
| 200 | N | TRP | E | 168 | −19.482 | −16.680 | −36.702 | 1.00 | 0.00 |
| 201 | CA | TRP | E | 168 | −18.171 | −16.656 | −36.081 | 1.00 | 0.00 |
| 202 | C | TRP | E | 168 | −17.073 | −17.106 | −36.999 | 1.00 | 0.00 |
| 203 | O | TRP | E | 168 | −17.321 | −17.635 | −38.072 | 1.00 | 0.00 |
| 204 | CB | TRP | E | 168 | −18.179 | −17.516 | −34.806 | 1.00 | 0.00 |
| 205 | CG | TRP | E | 168 | −18.954 | −16.783 | −33.754 | 1.00 | 0.00 |
| 206 | CD1 | TRP | E | 168 | −20.089 | −17.186 | −33.157 | 1.00 | 0.00 |
| 207 | CD2 | TRP | E | 168 | −18.596 | −15.442 | −33.177 | 1.00 | 0.00 |
| 208 | NE1 | TRP | E | 168 | −20.487 | −16.282 | −32.298 | 1.00 | 0.00 |
| 209 | CE2 | TRP | E | 168 | −19.637 | −15.236 | −32.283 | 1.00 | 0.00 |
| 210 | CE3 | TRP | E | 168 | −17.562 | −14.531 | −33.368 | 1.00 | 0.00 |
| 211 | CZ2 | TRP | E | 168 | −19.710 | −14.074 | −31.520 | 1.00 | 0.00 |
| 212 | CZ3 | TRP | E | 168 | −17.635 | −13.364 | −32.604 | 1.00 | 0.00 |
| 213 | CH2 | TRP | E | 168 | −18.681 | −13.138 | −31.693 | 1.00 | 0.00 |
| 214 | N | LEU | E | 169 | −15.833 | −16.878 | −36.532 | 1.00 | 0.00 |
| 215 | CA | LEU | E | 169 | −14.686 | −17.317 | −37.302 | 1.00 | 0.00 |
| 216 | C | LEU | E | 169 | −13.606 | −17.686 | −36.328 | 1.00 | 0.00 |
| 217 | O | LEU | E | 169 | −13.400 | −16.971 | −35.361 | 1.00 | 0.00 |
| 218 | CB | LEU | E | 169 | −14.189 | −16.187 | −38.224 | 1.00 | 0.00 |
| 219 | CG | LEU | E | 169 | −15.183 | −15.960 | −39.380 | 1.00 | 0.00 |
| 220 | CD1 | LEU | E | 169 | −14.764 | −14.723 | −40.198 | 1.00 | 0.00 |
| 221 | CD2 | LEU | E | 169 | −15.229 | −17.198 | −40.300 | 1.00 | 0.00 |
| 222 | N | LEU | E | 170 | −12.923 | −18.821 | −36.571 | 1.00 | 0.00 |
| 223 | CA | LEU | E | 170 | −11.932 | −19.252 | −35.605 | 1.00 | 0.00 |
| 224 | C | LEU | E | 170 | −10.737 | −18.341 | −35.619 | 1.00 | 0.00 |
| 225 | O | LEU | E | 170 | −9.909 | −18.428 | −36.511 | 1.00 | 0.00 |
| 226 | CB | LEU | E | 170 | −11.518 | −20.718 | −35.843 | 1.00 | 0.00 |
| 227 | CG | LEU | E | 170 | −10.521 | −21.166 | −34.752 | 1.00 | 0.00 |
| 228 | CD1 | LEU | E | 170 | −11.186 | −21.080 | −33.366 | 1.00 | 0.00 |
| 229 | CD2 | LEU | E | 170 | −10.082 | −22.619 | −35.013 | 1.00 | 0.00 |
| 230 | N | SER | E | 171 | −10.661 | −17.474 | −34.590 | 1.00 | 0.00 |
| 231 | CA | SER | E | 171 | −9.478 | −16.649 | −34.436 | 1.00 | 0.00 |
| 232 | C | SER | E | 171 | −8.303 | −17.556 | −34.198 | 1.00 | 0.00 |
| 233 | O | SER | E | 171 | −7.320 | −17.475 | −34.917 | 1.00 | 0.00 |
| 234 | CB | SER | E | 171 | −9.682 | −15.730 | −33.218 | 1.00 | 0.00 |
| 235 | OG | SER | E | 171 | −8.526 | −14.907 | −33.032 | 1.00 | 0.00 |
| 236 | N | PHE | E | 172 | −8.432 | −18.435 | −33.184 | 1.00 | 0.00 |
| 237 | CA | PHE | E | 172 | −7.375 | −19.400 | −32.947 | 1.00 | 0.00 |
| 238 | C | PHE | E | 172 | −7.833 | −20.470 | −31.996 | 1.00 | 0.00 |
| 239 | O | PHE | E | 172 | −8.882 | −20.353 | −31.381 | 1.00 | 0.00 |
| 240 | CB | PHE | E | 172 | −6.081 | −18.732 | −32.439 | 1.00 | 0.00 |
| 241 | CG | PHE | E | 172 | −6.331 | −17.994 | −31.126 | 1.00 | 0.00 |
| 242 | CD1 | PHE | E | 172 | −6.211 | −18.673 | −29.910 | 1.00 | 0.00 |
| 243 | CD2 | PHE | E | 172 | −6.676 | −16.639 | −31.139 | 1.00 | 0.00 |
| 244 | CE1 | PHE | E | 172 | −6.420 | −17.994 | −28.706 | 1.00 | 0.00 |
| 245 | CE2 | PHE | E | 172 | −6.870 | −15.955 | −29.936 | 1.00 | 0.00 |
| 246 | CZ | PHE | E | 172 | −6.749 | −16.636 | −28.721 | 1.00 | 0.00 |
| 247 | N | LYS | E | 173 | −7.011 | −21.529 | −31.885 | 1.00 | 0.00 |
| 248 | CA | LYS | E | 173 | −7.358 | −22.599 | −30.972 | 1.00 | 0.00 |
| 249 | C | LYS | E | 173 | −6.124 | −23.068 | −30.252 | 1.00 | 0.00 |
| 250 | O | LYS | E | 173 | −5.021 | −22.915 | −30.754 | 1.00 | 0.00 |
| 251 | CB | LYS | E | 173 | −8.036 | −23.750 | −31.741 | 1.00 | 0.00 |
| 252 | CG | LYS | E | 173 | −8.545 | −24.830 | −30.765 | 1.00 | 0.00 |
| 253 | CD | LYS | E | 173 | −9.117 | −26.002 | −31.581 | 1.00 | 0.00 |
| 254 | CE | LYS | E | 173 | −9.395 | −27.195 | −30.648 | 1.00 | 0.00 |
| 255 | NZ | LYS | E | 173 | −9.820 | −28.354 | −31.447 | 1.00 | 0.00 |
| 256 | N | ARG | E | 174 | −6.328 | −23.632 | −29.047 | 1.00 | 0.00 |
| 257 | CA | ARG | E | 174 | −5.180 | −24.057 | −28.270 | 1.00 | 0.00 |
| 258 | C | ARG | E | 174 | −5.564 | −25.256 | −27.453 | 1.00 | 0.00 |
| 259 | O | ARG | E | 174 | −6.513 | −25.195 | −26.688 | 1.00 | 0.00 |
| 260 | CB | ARG | E | 174 | −4.694 | −22.905 | −27.372 | 1.00 | 0.00 |
| 261 | CG | ARG | E | 174 | −3.308 | −23.238 | −26.786 | 1.00 | 0.00 |
| 262 | CD | ARG | E | 174 | −2.778 | −22.028 | −25.993 | 1.00 | 0.00 |
| 263 | NE | ARG | E | 174 | −1.477 | −22.347 | −25.434 | 1.00 | 0.00 |
| 264 | CZ | ARG | E | 174 | −1.194 | −22.107 | −24.185 | 1.00 | 0.00 |
| 265 | NH1 | ARG | E | 174 | −2.060 | −21.559 | −23.383 | 1.00 | 0.00 |
| 266 | NH2 | ARG | E | 174 | −0.019 | −22.424 | −23.728 | 1.00 | 0.00 |
| 267 | N | GLY | E | 175 | −4.816 | −26.360 | −27.634 | 1.00 | 0.00 |
| 268 | CA | GLY | E | 175 | −5.163 | −27.572 | −26.914 | 1.00 | 0.00 |
| 269 | C | GLY | E | 175 | −6.343 | −28.257 | −27.544 | 1.00 | 0.00 |
| 270 | O | GLY | E | 175 | −6.609 | −28.070 | −28.721 | 1.00 | 0.00 |
| 271 | N | SER | E | 176 | −7.056 | −29.065 | −26.736 | 1.00 | 0.00 |
| 272 | CA | SER | E | 176 | −8.187 | −29.791 | −27.288 | 1.00 | 0.00 |
| 273 | C | SER | E | 176 | −9.361 | −29.758 | −26.350 | 1.00 | 0.00 |
| 274 | O | SER | E | 176 | −10.367 | −30.393 | −26.626 | 1.00 | 0.00 |
| 275 | CB | SER | E | 176 | −7.770 | −31.244 | −27.581 | 1.00 | 0.00 |
| 276 | OG | SER | E | 176 | −7.335 | −31.875 | −26.372 | 1.00 | 0.00 |
| 277 | N | ALA | E | 177 | −9.227 | −29.012 | −25.235 | 1.00 | 0.00 |
| 278 | CA | ALA | E | 177 | −10.327 | −28.921 | −24.289 | 1.00 | 0.00 |
| 279 | C | ALA | E | 177 | −11.499 | −28.239 | −24.934 | 1.00 | 0.00 |
| 280 | O | ALA | E | 177 | −12.628 | −28.645 | −24.711 | 1.00 | 0.00 |
| 281 | CB | ALA | E | 177 | −9.862 | −28.102 | −23.072 | 1.00 | 0.00 |
| 282 | N | LEU | E | 178 | −11.225 | −27.201 | −25.746 | 1.00 | 0.00 |
| 283 | CA | LEU | E | 178 | −12.331 | −26.522 | −26.397 | 1.00 | 0.00 |
| 284 | C | LEU | E | 178 | −12.357 | −26.822 | −27.870 | 1.00 | 0.00 |
| 285 | O | LEU | E | 178 | −11.412 | −27.385 | −28.400 | 1.00 | 0.00 |
| 286 | CB | LEU | E | 178 | −12.218 | −25.003 | −26.163 | 1.00 | 0.00 |
| 287 | CG | LEU | E | 178 | −12.280 | −24.703 | −24.651 | 1.00 | 0.00 |
| 288 | CD1 | LEU | E | 178 | −12.047 | −23.199 | −24.407 | 1.00 | 0.00 |
| 289 | CD2 | LEU | E | 178 | −13.654 | −25.115 | −24.082 | 1.00 | 0.00 |
| 290 | N | GLU | E | 179 | −13.474 | −26.436 | −28.518 | 1.00 | 0.00 |
| 291 | CA | GLU | E | 179 | −13.594 | −26.683 | −29.943 | 1.00 | 0.00 |
| 292 | C | GLU | E | 179 | −14.691 | −25.837 | −30.526 | 1.00 | 0.00 |
| 293 | O | GLU | E | 179 | −15.538 | −25.341 | −29.800 | 1.00 | 0.00 |
| 294 | CB | GLU | E | 179 | −13.933 | −28.165 | −30.192 | 1.00 | 0.00 |
| 295 | CG | GLU | E | 179 | −12.650 | −28.946 | −30.538 | 1.00 | 0.00 |
| 296 | CD | GLU | E | 179 | −12.941 | −30.416 | −30.632 | 1.00 | 0.00 |
| 297 | OE1 | GLU | E | 179 | −13.850 | −30.797 | −31.418 | 1.00 | 0.00 |
| 298 | OE2 | GLU | E | 179 | −12.257 | −31.197 | −29.918 | 1.00 | 0.00 |
| 299 | N | GLU | E | 180 | −14.675 | −25.671 | −31.862 | 1.00 | 0.00 |
| 300 | CA | GLU | E | 180 | −15.775 | −24.960 | −32.486 | 1.00 | 0.00 |
| 301 | C | GLU | E | 180 | −16.721 | −25.961 | −33.090 | 1.00 | 0.00 |
| 302 | O | GLU | E | 180 | −16.290 | −27.019 | −33.522 | 1.00 | 0.00 |
| 303 | CB | GLU | E | 180 | −15.263 | −23.966 | −33.545 | 1.00 | 0.00 |
| 304 | CG | GLU | E | 180 | −16.419 | −23.075 | −34.043 | 1.00 | 0.00 |
| 305 | CD | GLU | E | 180 | −15.910 | −21.983 | −34.943 | 1.00 | 0.00 |
| 306 | OE1 | GLU | E | 180 | −15.018 | −22.264 | −35.788 | 1.00 | 0.00 |
| 307 | OE2 | GLU | E | 180 | −16.400 | −20.831 | −34.800 | 1.00 | 0.00 |
| 308 | N | LYS | E | 181 | −18.027 | −25.627 | −33.107 | 1.00 | 0.00 |
| 309 | CA | LYS | E | 181 | −18.981 | −26.577 | −33.645 | 1.00 | 0.00 |
| 310 | C | LYS | E | 181 | −20.299 | −25.906 | −33.892 | 1.00 | 0.00 |
| 311 | O | LYS | E | 181 | −20.996 | −25.563 | −32.950 | 1.00 | 0.00 |
| 312 | CB | LYS | E | 181 | −19.167 | −27.757 | −32.673 | 1.00 | 0.00 |
| 313 | CG | LYS | E | 181 | −20.057 | −28.824 | −33.343 | 1.00 | 0.00 |
| 314 | CD | LYS | E | 181 | −20.403 | −29.929 | −32.331 | 1.00 | 0.00 |
| 315 | CE | LYS | E | 181 | −21.354 | −30.944 | −32.994 | 1.00 | 0.00 |
| 316 | NZ | LYS | E | 181 | −21.619 | −32.050 | −32.061 | 1.00 | 0.00 |
| 317 | N | GLU | E | 182 | −20.632 | −25.741 | −35.189 | 1.00 | 0.00 |
| 318 | CA | GLU | E | 182 | −21.923 | −25.172 | −35.541 | 1.00 | 0.00 |
| 319 | C | GLU | E | 182 | −22.076 | −23.798 | −34.950 | 1.00 | 0.00 |
| 320 | O | GLU | E | 182 | −23.076 | −23.503 | −34.315 | 1.00 | 0.00 |
| 321 | CB | GLU | E | 182 | −23.065 | −26.128 | −35.143 | 1.00 | 0.00 |
| 322 | CG | GLU | E | 182 | −22.802 | −27.518 | −35.756 | 1.00 | 0.00 |
| 323 | CD | GLU | E | 182 | −23.869 | −28.480 | −35.321 | 1.00 | 0.00 |
| 324 | OE1 | GLU | E | 182 | −23.828 | −28.914 | −34.139 | 1.00 | 0.00 |
| 325 | OE2 | GLU | E | 182 | −24.742 | −28.811 | −36.166 | 1.00 | 0.00 |
| 326 | N | ASN | E | 183 | −21.043 | −22.957 | −35.164 | 1.00 | 0.00 |
| 327 | CA | ASN | E | 183 | −21.067 | −21.618 | −34.598 | 1.00 | 0.00 |
| 328 | C | ASN | E | 183 | −21.194 | −21.651 | −33.098 | 1.00 | 0.00 |
| 329 | O | ASN | E | 183 | −21.924 | −20.857 | −32.526 | 1.00 | 0.00 |
| 330 | CB | ASN | E | 183 | −22.176 | −20.779 | −35.258 | 1.00 | 0.00 |
| 331 | CG | ASN | E | 183 | −21.805 | −19.325 | −35.240 | 1.00 | 0.00 |
| 332 | OD1 | ASN | E | 183 | −22.502 | −18.528 | −34.633 | 1.00 | 0.00 |
| 333 | ND2 | ASN | E | 183 | −20.698 | −18.970 | −35.914 | 1.00 | 0.00 |
| 334 | N | LYS | E | 184 | −20.466 | −22.595 | −32.467 | 1.00 | 0.00 |
| 335 | CA | LYS | E | 184 | −20.547 | −22.708 | −31.022 | 1.00 | 0.00 |
| 336 | C | LYS | E | 184 | −19.254 | −23.210 | −30.443 | 1.00 | 0.00 |
| 337 | O | LYS | E | 184 | −18.359 | −23.620 | −31.166 | 1.00 | 0.00 |
| 338 | CB | LYS | E | 184 | −21.676 | −23.677 | −30.625 | 1.00 | 0.00 |
| 339 | CG | LYS | E | 184 | −23.054 | −23.032 | −30.869 | 1.00 | 0.00 |
| 340 | CD | LYS | E | 184 | −24.155 | −23.967 | −30.333 | 1.00 | 0.00 |
| 341 | CE | LYS | E | 184 | −24.154 | −25.300 | −31.108 | 1.00 | 0.00 |
| 342 | NZ | LYS | E | 184 | −24.616 | −25.085 | −32.488 | 1.00 | 0.00 |
| 343 | N | ILE | E | 185 | −19.165 | −23.169 | −29.100 | 1.00 | 0.00 |
| 344 | CA | ILE | E | 185 | −17.954 | −23.652 | −28.461 | 1.00 | 0.00 |
| 345 | C | ILE | E | 185 | −18.260 | −24.948 | −27.768 | 1.00 | 0.00 |
| 346 | O | ILE | E | 185 | −18.942 | −24.951 | −26.755 | 1.00 | 0.00 |
| 347 | CB | ILE | E | 185 | −17.424 | −22.600 | −27.466 | 1.00 | 0.00 |
| 348 | CG1 | ILE | E | 185 | −17.186 | −21.267 | −28.204 | 1.00 | 0.00 |
| 349 | CG2 | ILE | E | 185 | −16.092 | −23.096 | −26.869 | 1.00 | 0.00 |
| 350 | CD1 | ILE | E | 185 | −16.797 | −20.166 | −27.199 | 1.00 | 0.00 |
| 351 | N | LEU | E | 186 | −17.738 | −26.055 | −28.330 | 1.00 | 0.00 |
| 352 | CA | LEU | E | 186 | −17.976 | −27.341 | −27.700 | 1.00 | 0.00 |
| 353 | C | LEU | E | 186 | −17.029 | −27.579 | −26.556 | 1.00 | 0.00 |
| 354 | O | LEU | E | 186 | −15.851 | −27.270 | −26.651 | 1.00 | 0.00 |
| 355 | CB | LEU | E | 186 | −17.860 | −28.472 | −28.740 | 1.00 | 0.00 |
| 356 | CG | LEU | E | 186 | −18.142 | −29.840 | −28.089 | 1.00 | 0.00 |
| 357 | CD1 | LEU | E | 186 | −19.575 | −29.880 | −27.520 | 1.00 | 0.00 |
| 358 | CD2 | LEU | E | 186 | −17.978 | −30.947 | −29.149 | 1.00 | 0.00 |
| 359 | N | VAL | E | 187 | −17.573 | −28.141 | −25.460 | 1.00 | 0.00 |
| 360 | CA | VAL | E | 187 | −16.726 | −28.437 | −24.319 | 1.00 | 0.00 |
| 361 | C | VAL | E | 187 | −16.244 | −29.854 | −24.446 | 1.00 | 0.00 |
| 362 | O | VAL | E | 187 | −16.993 | −30.779 | −24.173 | 1.00 | 0.00 |
| 363 | CB | VAL | E | 187 | −17.514 | −28.258 | −23.004 | 1.00 | 0.00 |
| 364 | CG1 | VAL | E | 187 | −16.569 | −28.466 | −21.805 | 1.00 | 0.00 |
| 365 | CG2 | VAL | E | 187 | −18.100 | −26.836 | −22.933 | 1.00 | 0.00 |
| 366 | N | LYS | E | 188 | −14.973 | −29.998 | −24.864 | 1.00 | 0.00 |
| 367 | CA | LYS | E | 188 | −14.406 | −31.328 | −24.991 | 1.00 | 0.00 |
| 368 | C | LYS | E | 188 | −13.964 | −31.849 | −23.651 | 1.00 | 0.00 |
| 369 | O | LYS | E | 188 | −13.939 | −33.054 | −23.458 | 1.00 | 0.00 |
| 370 | CB | LYS | E | 188 | −13.207 | −31.282 | −25.961 | 1.00 | 0.00 |
| 371 | CG | LYS | E | 188 | −13.630 | −30.637 | −27.297 | 1.00 | 0.00 |
| 372 | CD | LYS | E | 188 | −14.825 | −31.395 | −27.910 | 1.00 | 0.00 |
| 373 | CE | LYS | E | 188 | −14.371 | −32.769 | −28.443 | 1.00 | 0.00 |
| 374 | NZ | LYS | E | 188 | −14.166 | −33.708 | −27.330 | 1.00 | 0.00 |
| 375 | N | GLU | E | 189 | −13.625 | −30.940 | −22.716 | 1.00 | 0.00 |
| 376 | CA | GLU | E | 189 | −13.189 | −31.402 | −21.411 | 1.00 | 0.00 |
| 377 | C | GLU | E | 189 | −13.898 | −30.659 | −20.315 | 1.00 | 0.00 |
| 378 | O | GLU | E | 189 | −14.115 | −29.463 | −20.426 | 1.00 | 0.00 |
| 379 | CB | GLU | E | 189 | −11.665 | −31.232 | −21.273 | 1.00 | 0.00 |
| 380 | CG | GLU | E | 189 | −10.941 | −32.153 | −22.275 | 1.00 | 0.00 |
| 381 | CD | GLU | E | 189 | −9.532 | −31.682 | −22.496 | 1.00 | 0.00 |
| 382 | OE1 | GLU | E | 189 | −8.819 | −31.438 | −21.485 | 1.00 | 0.00 |
| 383 | OE2 | GLU | E | 189 | −9.133 | −31.549 | −23.683 | 1.00 | 0.00 |
| 384 | N | THR | E | 190 | −14.261 | −31.395 | −19.247 | 1.00 | 0.00 |
| 385 | CA | THR | E | 190 | −14.913 | −30.737 | −18.129 | 1.00 | 0.00 |
| 386 | C | THR | E | 190 | −13.910 | −29.937 | −17.340 | 1.00 | 0.00 |
| 387 | O | THR | E | 190 | −12.717 | −30.171 | −17.452 | 1.00 | 0.00 |
| 388 | CB | THR | E | 190 | −15.602 | −31.786 | −17.232 | 1.00 | 0.00 |
| 389 | OG1 | THR | E | 190 | −16.441 | −32.636 | −18.020 | 1.00 | 0.00 |
| 390 | CG2 | THR | E | 190 | −16.455 | −31.091 | −16.154 | 1.00 | 0.00 |
| 391 | N | GLY | E | 191 | −14.406 | −28.974 | −16.542 | 1.00 | 0.00 |
| 392 | CA | GLY | E | 191 | −13.488 | −28.170 | −15.755 | 1.00 | 0.00 |
| 393 | C | GLY | E | 191 | −13.925 | −26.732 | −15.728 | 1.00 | 0.00 |
| 394 | O | GLY | E | 191 | −14.977 | −26.396 | −16.247 | 1.00 | 0.00 |
| 395 | N | TYR | E | 192 | −13.094 | −25.872 | −15.110 | 1.00 | 0.00 |
| 396 | CA | TYR | E | 192 | −13.448 | −24.465 | −15.080 | 1.00 | 0.00 |
| 397 | C | TYR | E | 192 | −13.010 | −23.773 | −16.340 | 1.00 | 0.00 |
| 398 | O | TYR | E | 192 | −12.107 | −24.235 | −17.021 | 1.00 | 0.00 |
| 399 | CB | TYR | E | 192 | −12.830 | −23.767 | −13.855 | 1.00 | 0.00 |
| 400 | CG | TYR | E | 192 | −13.561 | −24.219 | −12.597 | 1.00 | 0.00 |
| 401 | CD1 | TYR | E | 192 | −14.518 | −23.385 | −12.013 | 1.00 | 0.00 |
| 402 | CD2 | TYR | E | 192 | −13.274 | −25.464 | −12.032 | 1.00 | 0.00 |
| 403 | CE1 | TYR | E | 192 | −15.186 | −23.797 | −10.857 | 1.00 | 0.00 |
| 404 | CE2 | TYR | E | 192 | −13.946 | −25.874 | −10.878 | 1.00 | 0.00 |
| 405 | CZ | TYR | E | 192 | −14.900 | −25.041 | −10.288 | 1.00 | 0.00 |
| 406 | OH | TYR | E | 192 | −15.561 | −25.448 | −9.135 | 1.00 | 0.00 |
| 407 | N | PHE | E | 193 | −13.679 | −22.646 | −16.646 | 1.00 | 0.00 |
| 408 | CA | PHE | E | 193 | −13.327 | −21.932 | −17.858 | 1.00 | 0.00 |
| 409 | C | PHE | E | 193 | −13.613 | −20.466 | −17.710 | 1.00 | 0.00 |
| 410 | O | PHE | E | 193 | −14.603 | −20.086 | −17.105 | 1.00 | 0.00 |
| 411 | CB | PHE | E | 193 | −14.157 | −22.443 | −19.051 | 1.00 | 0.00 |
| 412 | CG | PHE | E | 193 | −13.794 | −23.879 | −19.415 | 1.00 | 0.00 |
| 413 | CD1 | PHE | E | 193 | −14.497 | −24.956 | −18.867 | 1.00 | 0.00 |
| 414 | CD2 | PHE | E | 193 | −12.747 | −24.108 | −20.311 | 1.00 | 0.00 |
| 415 | CE1 | PHE | E | 193 | −14.125 | −26.265 | −19.190 | 1.00 | 0.00 |
| 416 | CE2 | PHE | E | 193 | −12.396 | −25.415 | −20.654 | 1.00 | 0.00 |
| 417 | CZ | PHE | E | 193 | −13.087 | −26.495 | −20.097 | 1.00 | 0.00 |
| 418 | N | PHE | E | 194 | −12.718 | −19.645 | −18.289 | 1.00 | 0.00 |
| 419 | CA | PHE | E | 194 | −12.993 | −18.224 | −18.317 | 1.00 | 0.00 |
| 420 | C | PHE | E | 194 | −13.657 | −17.928 | −19.630 | 1.00 | 0.00 |
| 421 | O | PHE | E | 194 | −13.110 | −18.254 | −20.672 | 1.00 | 0.00 |
| 422 | CB | PHE | E | 194 | −11.699 | −17.399 | −18.169 | 1.00 | 0.00 |
| 423 | CG | PHE | E | 194 | −12.020 | −15.921 | −18.384 | 1.00 | 0.00 |
| 424 | CD1 | PHE | E | 194 | −12.674 | −15.194 | −17.386 | 1.00 | 0.00 |
| 425 | CD2 | PHE | E | 194 | −11.672 | −15.298 | −19.586 | 1.00 | 0.00 |
| 426 | CE1 | PHE | E | 194 | −12.994 | −13.850 | −17.594 | 1.00 | 0.00 |
| 427 | CE2 | PHE | E | 194 | −11.989 | −13.953 | −19.796 | 1.00 | 0.00 |
| 428 | CZ | PHE | E | 194 | −12.647 | −13.230 | −18.797 | 1.00 | 0.00 |
| 429 | N | ILE | E | 195 | −14.850 | −17.309 | −19.571 | 1.00 | 0.00 |
| 430 | CA | ILE | E | 195 | −15.531 | −17.001 | −20.815 | 1.00 | 0.00 |
| 431 | C | ILE | E | 195 | −15.650 | −15.512 | −20.998 | 1.00 | 0.00 |
| 432 | O | ILE | E | 195 | −15.512 | −14.763 | −20.043 | 1.00 | 0.00 |
| 433 | CB | ILE | E | 195 | −16.903 | −17.701 | −20.865 | 1.00 | 0.00 |
| 434 | CG1 | ILE | E | 195 | −16.714 | −19.204 | −20.573 | 1.00 | 0.00 |
| 435 | CG2 | ILE | E | 195 | −17.542 | −17.519 | −22.258 | 1.00 | 0.00 |
| 436 | CD1 | ILE | E | 195 | −18.084 | −19.891 | −20.418 | 1.00 | 0.00 |
| 437 | N | TYR | E | 196 | −15.893 | −15.089 | −22.254 | 1.00 | 0.00 |
| 438 | CA | TYR | E | 196 | −15.976 | −13.664 | −22.518 | 1.00 | 0.00 |
| 439 | C | TYR | E | 196 | −16.542 | −13.421 | −23.891 | 1.00 | 0.00 |
| 440 | O | TYR | E | 196 | −16.619 | −14.338 | −24.694 | 1.00 | 0.00 |
| 441 | CB | TYR | E | 196 | −14.590 | −13.001 | −22.392 | 1.00 | 0.00 |
| 442 | CG | TYR | E | 196 | −13.591 | −13.696 | −23.313 | 1.00 | 0.00 |
| 443 | CD1 | TYR | E | 196 | −13.438 | −13.260 | −24.632 | 1.00 | 0.00 |
| 444 | CD2 | TYR | E | 196 | −12.835 | −14.771 | −22.838 | 1.00 | 0.00 |
| 445 | CE1 | TYR | E | 196 | −12.529 | −13.902 | −25.477 | 1.00 | 0.00 |
| 446 | CE2 | TYR | E | 196 | −11.920 | −15.406 | −23.682 | 1.00 | 0.00 |
| 447 | CZ | TYR | E | 196 | −11.771 | −14.975 | −25.003 | 1.00 | 0.00 |
| 448 | OH | TYR | E | 196 | −10.869 | −15.615 | −25.845 | 1.00 | 0.00 |
| 449 | N | GLY | E | 197 | −16.943 | −12.162 | −24.151 | 1.00 | 0.00 |
| 450 | CA | GLY | E | 197 | −17.513 | −11.855 | −25.449 | 1.00 | 0.00 |
| 451 | C | GLY | E | 197 | −17.768 | −10.379 | −25.569 | 1.00 | 0.00 |
| 452 | O | GLY | E | 197 | −18.014 | −9.716 | −24.573 | 1.00 | 0.00 |
| 453 | N | GLN | E | 198 | −17.712 | −9.867 | −26.813 | 1.00 | 0.00 |
| 454 | CA | GLN | E | 198 | −17.999 | −8.458 | −27.000 | 1.00 | 0.00 |
| 455 | C | GLN | E | 198 | −18.765 | −8.227 | −28.272 | 1.00 | 0.00 |
| 456 | O | GLN | E | 198 | −18.592 | −8.956 | −29.236 | 1.00 | 0.00 |
| 457 | CB | GLN | E | 198 | −16.677 | −7.673 | −27.023 | 1.00 | 0.00 |
| 458 | CG | GLN | E | 198 | −16.938 | −6.199 | −26.656 | 1.00 | 0.00 |
| 459 | CD | GLN | E | 198 | −15.637 | −5.437 | −26.626 | 1.00 | 0.00 |
| 460 | OE1 | GLN | E | 198 | −15.240 | −4.967 | −25.571 | 1.00 | 0.00 |
| 461 | NE2 | GLN | E | 198 | −14.968 | −5.298 | −27.786 | 1.00 | 0.00 |
| 462 | N | VAL | E | 199 | −19.631 | −7.195 | −28.259 | 1.00 | 0.00 |
| 463 | CA | VAL | E | 199 | −20.442 | −6.942 | −29.436 | 1.00 | 0.00 |
| 464 | C | VAL | E | 199 | −20.609 | −5.462 | −29.631 | 1.00 | 0.00 |
| 465 | O | VAL | E | 199 | −20.844 | −4.739 | −28.676 | 1.00 | 0.00 |
| 466 | CB | VAL | E | 199 | −21.828 | −7.607 | −29.307 | 1.00 | 0.00 |
| 467 | CG1 | VAL | E | 199 | −22.618 | −7.413 | −30.617 | 1.00 | 0.00 |
| 468 | CG2 | VAL | E | 199 | −21.698 | −9.113 | −29.010 | 1.00 | 0.00 |
| 469 | N | LEU | E | 200 | −20.479 | −5.017 | −30.895 | 1.00 | 0.00 |
| 470 | CA | LEU | E | 200 | −20.657 | −3.601 | −31.156 | 1.00 | 0.00 |
| 471 | C | LEU | E | 200 | −22.035 | −3.371 | −31.709 | 1.00 | 0.00 |
| 472 | O | LEU | E | 200 | −22.284 | −3.657 | −32.870 | 1.00 | 0.00 |
| 473 | CB | LEU | E | 200 | −19.583 | −3.113 | −32.148 | 1.00 | 0.00 |
| 474 | CG | LEU | E | 200 | −19.806 | −1.623 | −32.479 | 1.00 | 0.00 |
| 475 | CD1 | LEU | E | 200 | −19.740 | −0.766 | −31.198 | 1.00 | 0.00 |
| 476 | CD2 | LEU | E | 200 | −18.723 | −1.157 | −33.469 | 1.00 | 0.00 |
| 477 | N | TYR | E | 201 | −22.930 | −2.835 | −30.858 | 1.00 | 0.00 |
| 478 | CA | TYR | E | 201 | −24.264 | −2.537 | −31.346 | 1.00 | 0.00 |
| 479 | C | TYR | E | 201 | −24.273 | −1.251 | −32.124 | 1.00 | 0.00 |
| 480 | O | TYR | E | 201 | −23.403 | −0.416 | −31.931 | 1.00 | 0.00 |
| 481 | CB | TYR | E | 201 | −25.289 | −2.541 | −30.197 | 1.00 | 0.00 |
| 482 | CG | TYR | E | 201 | −25.287 | −3.942 | −29.598 | 1.00 | 0.00 |
| 483 | CD1 | TYR | E | 201 | −24.378 | −4.253 | −28.584 | 1.00 | 0.00 |
| 484 | CD2 | TYR | E | 201 | −26.176 | −4.911 | −30.070 | 1.00 | 0.00 |
| 485 | CE1 | TYR | E | 201 | −24.273 | −5.569 | −28.128 | 1.00 | 0.00 |
| 486 | CE2 | TYR | E | 201 | −26.071 | −6.226 | −29.609 | 1.00 | 0.00 |
| 487 | CZ | TYR | E | 201 | −25.084 | −6.565 | −28.678 | 1.00 | 0.00 |
| 488 | OH | TYR | E | 201 | −24.904 | −7.892 | −28.307 | 1.00 | 0.00 |
| 489 | N | THR | E | 202 | −25.259 | −1.110 | −33.031 | 1.00 | 0.00 |
| 490 | CA | THR | E | 202 | −25.255 | 0.057 | −33.896 | 1.00 | 0.00 |
| 491 | C | THR | E | 202 | −26.649 | 0.575 | −34.122 | 1.00 | 0.00 |
| 492 | O | THR | E | 202 | −26.806 | 1.614 | −34.744 | 1.00 | 0.00 |
| 493 | CB | THR | E | 202 | −24.576 | −0.292 | −35.237 | 1.00 | 0.00 |
| 494 | OG1 | THR | E | 202 | −25.198 | −1.446 | −35.815 | 1.00 | 0.00 |
| 495 | CG2 | THR | E | 202 | −23.079 | −0.577 | −35.019 | 1.00 | 0.00 |
| 496 | N | ASP | E | 203 | −27.673 | −0.136 | −33.612 | 1.00 | 0.00 |
| 497 | CA | ASP | E | 203 | −29.015 | 0.405 | −33.734 | 1.00 | 0.00 |
| 498 | C | ASP | E | 203 | −29.280 | 1.402 | −32.638 | 1.00 | 0.00 |
| 499 | O | ASP | E | 203 | −28.627 | 1.362 | −31.608 | 1.00 | 0.00 |
| 500 | CB | ASP | E | 203 | −30.080 | −0.705 | −33.796 | 1.00 | 0.00 |
| 501 | CG | ASP | E | 203 | −29.945 | −1.648 | −32.636 | 1.00 | 0.00 |
| 502 | OD1 | ASP | E | 203 | −29.119 | −2.596 | −32.732 | 1.00 | 0.00 |
| 503 | OD2 | ASP | E | 203 | −30.679 | −1.447 | −31.632 | 1.00 | 0.00 |
| 504 | N | LYS | E | 204 | −30.235 | 2.320 | −32.884 | 1.00 | 0.00 |
| 505 | CA | LYS | E | 204 | −30.396 | 3.439 | −31.974 | 1.00 | 0.00 |
| 506 | C | LYS | E | 204 | −31.577 | 3.313 | −31.045 | 1.00 | 0.00 |
| 507 | O | LYS | E | 204 | −32.213 | 2.274 | −30.974 | 1.00 | 0.00 |
| 508 | CB | LYS | E | 204 | −30.550 | 4.684 | −32.871 | 1.00 | 0.00 |
| 509 | CG | LYS | E | 204 | −31.885 | 4.639 | −33.641 | 1.00 | 0.00 |
| 510 | CD | LYS | E | 204 | −31.956 | 5.828 | −34.615 | 1.00 | 0.00 |
| 511 | CE | LYS | E | 204 | −33.426 | 6.098 | −34.985 | 1.00 | 0.00 |
| 512 | NZ | LYS | E | 204 | −33.533 | 7.434 | −35.588 | 1.00 | 0.00 |
| 513 | N | THR | E | 205 | −31.849 | 4.434 | −30.343 | 1.00 | 0.00 |
| 514 | CA | THR | E | 205 | −33.048 | 4.538 | −29.530 | 1.00 | 0.00 |
| 515 | C | THR | E | 205 | −33.008 | 3.765 | −28.246 | 1.00 | 0.00 |
| 516 | O | THR | E | 205 | −32.982 | 4.388 | −27.196 | 1.00 | 0.00 |
| 517 | CB | THR | E | 205 | −34.381 | 4.444 | −30.297 | 1.00 | 0.00 |
| 518 | OG1 | THR | E | 205 | −34.706 | 3.087 | −30.619 | 1.00 | 0.00 |
| 519 | CG2 | THR | E | 205 | −34.324 | 5.324 | −31.561 | 1.00 | 0.00 |
| 520 | N | TYR | E | 206 | −33.009 | 2.419 | −28.303 | 1.00 | 0.00 |
| 521 | CA | TYR | E | 206 | −33.028 | 1.693 | −27.045 | 1.00 | 0.00 |
| 522 | C | TYR | E | 206 | −31.862 | 0.755 | −26.886 | 1.00 | 0.00 |
| 523 | O | TYR | E | 206 | −31.080 | 0.574 | −27.805 | 1.00 | 0.00 |
| 524 | CB | TYR | E | 206 | −34.385 | 0.992 | −26.835 | 1.00 | 0.00 |
| 525 | CG | TYR | E | 206 | −34.620 | 0.700 | −25.355 | 1.00 | 0.00 |
| 526 | CD1 | TYR | E | 206 | −35.375 | −0.414 | −24.976 | 1.00 | 0.00 |
| 527 | CD2 | TYR | E | 206 | −34.076 | 1.541 | −24.380 | 1.00 | 0.00 |
| 528 | CE1 | TYR | E | 206 | −35.561 | −0.697 | −23.620 | 1.00 | 0.00 |
| 529 | CE2 | TYR | E | 206 | −34.240 | 1.238 | −23.027 | 1.00 | 0.00 |
| 530 | CZ | TYR | E | 206 | −34.984 | 0.119 | −22.643 | 1.00 | 0.00 |
| 531 | OH | TYR | E | 206 | −35.150 | −0.179 | −21.294 | 1.00 | 0.00 |
| 532 | N | ALA | E | 207 | −31.754 | 0.170 | −25.677 | 1.00 | 0.00 |
| 533 | CA | ALA | E | 207 | −30.587 | −0.634 | −25.363 | 1.00 | 0.00 |
| 534 | C | ALA | E | 207 | −30.445 | −1.867 | −26.211 | 1.00 | 0.00 |
| 535 | O | ALA | E | 207 | −31.375 | −2.273 | −26.889 | 1.00 | 0.00 |
| 536 | CB | ALA | E | 207 | −30.612 | −1.024 | −23.875 | 1.00 | 0.00 |
| 537 | N | MET | E | 208 | −29.234 | −2.453 | −26.146 | 1.00 | 0.00 |
| 538 | CA | MET | E | 208 | −28.962 | −3.663 | −26.900 | 1.00 | 0.00 |
| 539 | C | MET | E | 208 | −27.993 | −4.509 | −26.118 | 1.00 | 0.00 |
| 540 | O | MET | E | 208 | −27.399 | −4.029 | −25.165 | 1.00 | 0.00 |
| 541 | CB | MET | E | 208 | −28.329 | −3.313 | −28.262 | 1.00 | 0.00 |
| 542 | CG | MET | E | 208 | −29.362 | −2.643 | −29.189 | 1.00 | 0.00 |
| 543 | SD | MET | E | 208 | −30.524 | −3.927 | −29.742 | 1.00 | 0.00 |
| 544 | CE | MET | E | 208 | −29.390 | −4.895 | −30.784 | 1.00 | 0.00 |
| 545 | N | GLY | E | 209 | −27.829 | −5.785 | −26.515 | 1.00 | 0.00 |
| 546 | CA | GLY | E | 209 | −26.909 | −6.626 | −25.769 | 1.00 | 0.00 |
| 547 | C | GLY | E | 209 | −26.924 | −8.046 | −26.257 | 1.00 | 0.00 |
| 548 | O | GLY | E | 209 | −27.546 | −8.354 | −27.263 | 1.00 | 0.00 |
| 549 | N | HIS | E | 210 | −26.210 | −8.914 | −25.516 | 1.00 | 0.00 |
| 550 | CA | HIS | E | 210 | −26.110 | −10.294 | −25.955 | 1.00 | 0.00 |
| 551 | C | HIS | E | 210 | −26.063 | −11.235 | −24.784 | 1.00 | 0.00 |
| 552 | O | HIS | E | 210 | −25.992 | −10.802 | −23.645 | 1.00 | 0.00 |
| 553 | CB | HIS | E | 210 | −24.878 | −10.487 | −26.861 | 1.00 | 0.00 |
| 554 | CG | HIS | E | 210 | −23.629 | −9.981 | −26.196 | 1.00 | 0.00 |
| 555 | ND1 | HIS | E | 210 | −23.369 | −8.698 | −26.092 | 1.00 | 0.00 |
| 556 | CD2 | HIS | E | 210 | −22.672 | −10.755 | −25.650 | 1.00 | 0.00 |
| 557 | CE1 | HIS | E | 210 | −22.236 | −8.581 | −25.476 | 1.00 | 0.00 |
| 558 | NE2 | HIS | E | 210 | −21.786 | −9.722 | −25.195 | 1.00 | 0.00 |
| 559 | N | LEU | E | 211 | −26.111 | −12.546 | −25.089 | 1.00 | 0.00 |
| 560 | CA | LEU | E | 211 | −26.131 | −13.519 | −24.011 | 1.00 | 0.00 |
| 561 | C | LEU | E | 211 | −25.126 | −14.607 | −24.265 | 1.00 | 0.00 |
| 562 | O | LEU | E | 211 | −24.978 | −15.061 | −25.389 | 1.00 | 0.00 |
| 563 | CB | LEU | E | 211 | −27.529 | −14.159 | −23.894 | 1.00 | 0.00 |
| 564 | CG | LEU | E | 211 | −28.629 | −13.080 | −23.896 | 1.00 | 0.00 |
| 565 | CD1 | LEU | E | 211 | −30.004 | −13.757 | −24.050 | 1.00 | 0.00 |
| 566 | CD2 | LEU | E | 211 | −28.590 | −12.280 | −22.579 | 1.00 | 0.00 |
| 567 | N | ILE | E | 212 | −24.426 | −15.032 | −23.198 | 1.00 | 0.00 |
| 568 | CA | ILE | E | 212 | −23.501 | −16.138 | −23.362 | 1.00 | 0.00 |
| 569 | C | ILE | E | 212 | −24.137 | −17.349 | −22.743 | 1.00 | 0.00 |
| 570 | O | ILE | E | 212 | −23.910 | −17.647 | −21.580 | 1.00 | 0.00 |
| 571 | CB | ILE | E | 212 | −22.125 | −15.772 | −22.767 | 1.00 | 0.00 |
| 572 | CG1 | ILE | E | 212 | −21.527 | −14.611 | −23.589 | 1.00 | 0.00 |
| 573 | CG2 | ILE | E | 212 | −21.188 | −16.997 | −22.816 | 1.00 | 0.00 |
| 574 | CD1 | ILE | E | 212 | −20.135 | −14.217 | −23.059 | 1.00 | 0.00 |
| 575 | N | GLN | E | 213 | −24.952 | −18.034 | −23.566 | 1.00 | 0.00 |
| 576 | CA | GLN | E | 213 | −25.683 | −19.176 | −23.054 | 1.00 | 0.00 |
| 577 | C | GLN | E | 213 | −24.838 | −20.412 | −22.897 | 1.00 | 0.00 |
| 578 | O | GLN | E | 213 | −23.672 | −20.441 | −23.261 | 1.00 | 0.00 |
| 579 | CB | GLN | E | 213 | −26.856 | −19.474 | −24.005 | 1.00 | 0.00 |
| 580 | CG | GLN | E | 213 | −27.919 | −18.365 | −23.876 | 1.00 | 0.00 |
| 581 | CD | GLN | E | 213 | −28.714 | −18.251 | −25.148 | 1.00 | 0.00 |
| 582 | OE1 | GLN | E | 213 | −28.170 | −18.388 | −26.231 | 1.00 | 0.00 |
| 583 | NE2 | GLN | E | 213 | −30.029 | −18.000 | −25.020 | 1.00 | 0.00 |
| 584 | N | ARG | E | 214 | −25.476 | −21.457 | −22.337 | 1.00 | 0.00 |
| 585 | CA | ARG | E | 214 | −24.779 | −22.715 | −22.159 | 1.00 | 0.00 |
| 586 | C | ARG | E | 214 | −25.730 | −23.834 | −22.472 | 1.00 | 0.00 |
| 587 | O | ARG | E | 214 | −26.697 | −24.032 | −21.753 | 1.00 | 0.00 |
| 588 | CB | ARG | E | 214 | −24.309 | −22.812 | −20.696 | 1.00 | 0.00 |
| 589 | CG | ARG | E | 214 | −23.699 | −24.203 | −20.434 | 1.00 | 0.00 |
| 590 | CD | ARG | E | 214 | −23.278 | −24.326 | −18.956 | 1.00 | 0.00 |
| 591 | NE | ARG | E | 214 | −22.947 | −25.705 | −18.635 | 1.00 | 0.00 |
| 592 | CZ | ARG | E | 214 | −23.386 | −26.277 | −17.549 | 1.00 | 0.00 |
| 593 | NH1 | ARG | E | 214 | −24.134 | −25.639 | −16.697 | 1.00 | 0.00 |
| 594 | NH2 | ARG | E | 214 | −23.073 | −27.515 | −17.305 | 1.00 | 0.00 |
| 595 | N | LYS | E | 215 | −25.434 | −24.575 | −23.557 | 1.00 | 0.00 |
| 596 | CA | LYS | E | 215 | −26.271 | −25.713 | −23.871 | 1.00 | 0.00 |
| 597 | C | LYS | E | 215 | −25.888 | −26.842 | −22.960 | 1.00 | 0.00 |
| 598 | O | LYS | E | 215 | −24.897 | −27.517 | −23.194 | 1.00 | 0.00 |
| 599 | CB | LYS | E | 215 | −26.082 | −26.098 | −25.350 | 1.00 | 0.00 |
| 600 | CG | LYS | E | 215 | −27.207 | −27.065 | −25.774 | 1.00 | 0.00 |
| 601 | CD | LYS | E | 215 | −28.573 | −26.384 | −25.579 | 1.00 | 0.00 |
| 602 | CE | LYS | E | 215 | −29.697 | −27.375 | −25.932 | 1.00 | 0.00 |
| 603 | NZ | LYS | E | 215 | −31.004 | −26.760 | −25.650 | 1.00 | 0.00 |
| 604 | N | LYS | E | 216 | −26.703 | −27.029 | −21.905 | 1.00 | 0.00 |
| 605 | CA | LYS | E | 216 | −26.393 | −28.065 | −20.936 | 1.00 | 0.00 |
| 606 | C | LYS | E | 216 | −26.470 | −29.434 | −21.552 | 1.00 | 0.00 |
| 607 | O | LYS | E | 216 | −27.401 | −29.731 | −22.283 | 1.00 | 0.00 |
| 608 | CB | LYS | E | 216 | −27.383 | −27.994 | −19.760 | 1.00 | 0.00 |
| 609 | CG | LYS | E | 216 | −27.203 | −26.667 | −18.998 | 1.00 | 0.00 |
| 610 | CD | LYS | E | 216 | −28.478 | −26.355 | −18.189 | 1.00 | 0.00 |
| 611 | CE | LYS | E | 216 | −28.727 | −27.458 | −17.142 | 1.00 | 0.00 |
| 612 | NZ | LYS | E | 216 | −29.942 | −27.145 | −16.378 | 1.00 | 0.00 |
| 613 | N | VAL | E | 217 | −25.463 | −30.271 | −21.237 | 1.00 | 0.00 |
| 614 | CA | VAL | E | 217 | −25.514 | −31.646 | −21.699 | 1.00 | 0.00 |
| 615 | C | VAL | E | 217 | −26.502 | −32.394 | −20.846 | 1.00 | 0.00 |
| 616 | O | VAL | E | 217 | −27.341 | −33.105 | −21.376 | 1.00 | 0.00 |
| 617 | CB | VAL | E | 217 | −24.101 | −32.268 | −21.641 | 1.00 | 0.00 |
| 618 | CG1 | VAL | E | 217 | −23.570 | −32.309 | −20.196 | 1.00 | 0.00 |
| 619 | CG2 | VAL | E | 217 | −24.123 | −33.704 | −22.201 | 1.00 | 0.00 |
| 620 | N | HIS | E | 218 | −26.406 | −32.207 | −19.515 | 1.00 | 0.00 |
| 621 | CA | HIS | E | 218 | −27.368 | −32.853 | −18.643 | 1.00 | 0.00 |
| 622 | C | HIS | E | 218 | −28.385 | −31.851 | −18.174 | 1.00 | 0.00 |
| 623 | O | HIS | E | 218 | −28.073 | −30.678 | −18.036 | 1.00 | 0.00 |
| 624 | CB | HIS | E | 218 | −26.658 | −33.480 | −17.427 | 1.00 | 0.00 |
| 625 | CG | HIS | E | 218 | −25.457 | −34.278 | −17.851 | 1.00 | 0.00 |
| 626 | ND1 | HIS | E | 218 | −24.336 | −34.250 | −17.165 | 1.00 | 0.00 |
| 627 | CD2 | HIS | E | 218 | −25.389 | −35.076 | −18.933 | 1.00 | 0.00 |
| 628 | CE1 | HIS | E | 218 | −23.495 | −35.025 | −17.770 | 1.00 | 0.00 |
| 629 | NE2 | HIS | E | 218 | −24.034 | −35.528 | −18.789 | 1.00 | 0.00 |
| 630 | N | VAL | E | 219 | −29.616 | −32.341 | −17.936 | 1.00 | 0.00 |
| 631 | CA | VAL | E | 219 | −30.646 | −31.457 | −17.423 | 1.00 | 0.00 |
| 632 | C | VAL | E | 219 | −31.539 | −32.247 | −16.506 | 1.00 | 0.00 |
| 633 | O | VAL | E | 219 | −31.746 | −33.430 | −16.726 | 1.00 | 0.00 |
| 634 | CB | VAL | E | 219 | −31.460 | −30.789 | −18.551 | 1.00 | 0.00 |
| 635 | CG1 | VAL | E | 219 | −32.457 | −29.784 | −17.940 | 1.00 | 0.00 |
| 636 | CG2 | VAL | E | 219 | −30.533 | −30.047 | −19.534 | 1.00 | 0.00 |
| 637 | N | PHE | E | 220 | −32.060 | −31.590 | −15.453 | 1.00 | 0.00 |
| 638 | CA | PHE | E | 220 | −32.865 | −32.333 | −14.499 | 1.00 | 0.00 |
| 639 | C | PHE | E | 220 | −33.940 | −31.467 | −13.905 | 1.00 | 0.00 |
| 640 | O | PHE | E | 220 | −33.863 | −30.251 | −13.994 | 1.00 | 0.00 |
| 641 | CB | PHE | E | 220 | −31.985 | −32.888 | −13.362 | 1.00 | 0.00 |
| 642 | CG | PHE | E | 220 | −30.833 | −33.709 | −13.934 | 1.00 | 0.00 |
| 643 | CD1 | PHE | E | 220 | −29.610 | −33.098 | −14.230 | 1.00 | 0.00 |
| 644 | CD2 | PHE | E | 220 | −31.006 | −35.075 | −14.168 | 1.00 | 0.00 |
| 645 | CE1 | PHE | E | 220 | −28.579 | −33.844 | −14.809 | 1.00 | 0.00 |
| 646 | CE2 | PHE | E | 220 | −29.972 | −35.824 | −14.735 | 1.00 | 0.00 |
| 647 | CZ | PHE | E | 220 | −28.761 | −35.206 | −15.062 | 1.00 | 0.00 |
| 648 | N | GLY | E | 221 | −34.945 | −32.133 | −13.300 | 1.00 | 0.00 |
| 649 | CA | GLY | E | 221 | −36.032 | −31.403 | −12.670 | 1.00 | 0.00 |
| 650 | C | GLY | E | 221 | −36.731 | −30.513 | −13.657 | 1.00 | 0.00 |
| 651 | O | GLY | E | 221 | −37.012 | −30.942 | −14.765 | 1.00 | 0.00 |
| 652 | N | ASP | E | 222 | −37.001 | −29.257 | −13.253 | 1.00 | 0.00 |
| 653 | CA | ASP | E | 222 | −37.626 | −28.341 | −14.194 | 1.00 | 0.00 |
| 654 | C | ASP | E | 222 | −36.604 | −27.386 | −14.751 | 1.00 | 0.00 |
| 655 | O | ASP | E | 222 | −36.929 | −26.261 | −15.099 | 1.00 | 0.00 |
| 656 | CB | ASP | E | 222 | −38.779 | −27.591 | −13.498 | 1.00 | 0.00 |
| 657 | CG | ASP | E | 222 | −39.689 | −28.561 | −12.800 | 1.00 | 0.00 |
| 658 | OD1 | ASP | E | 222 | −39.504 | −28.770 | −11.571 | 1.00 | 0.00 |
| 659 | OD2 | ASP | E | 222 | −40.602 | −29.106 | −13.476 | 1.00 | 0.00 |
| 660 | N | GLU | E | 223 | −35.340 | −27.846 | −14.825 | 1.00 | 0.00 |
| 661 | CA | GLU | E | 223 | −34.302 | −26.964 | −15.327 | 1.00 | 0.00 |
| 662 | C | GLU | E | 223 | −34.325 | −26.917 | −16.827 | 1.00 | 0.00 |
| 663 | O | GLU | E | 223 | −34.894 | −27.792 | −17.460 | 1.00 | 0.00 |
| 664 | CB | GLU | E | 223 | −32.918 | −27.418 | −14.830 | 1.00 | 0.00 |
| 665 | CG | GLU | E | 223 | −32.865 | −27.317 | −13.294 | 1.00 | 0.00 |
| 666 | CD | GLU | E | 223 | −31.467 | −26.987 | −12.859 | 1.00 | 0.00 |
| 667 | OE1 | GLU | E | 223 | −30.595 | −27.894 | −12.931 | 1.00 | 0.00 |
| 668 | OE2 | GLU | E | 223 | −31.239 | −25.823 | −12.434 | 1.00 | 0.00 |
| 669 | N | LEU | E | 224 | −33.700 | −25.868 | −17.391 | 1.00 | 0.00 |
| 670 | CA | LEU | E | 224 | −33.696 | −25.756 | −18.838 | 1.00 | 0.00 |
| 671 | C | LEU | E | 224 | −32.442 | −26.339 | −19.425 | 1.00 | 0.00 |
| 672 | O | LEU | E | 224 | −31.459 | −26.516 | −18.725 | 1.00 | 0.00 |
| 673 | CB | LEU | E | 224 | −33.829 | −24.280 | −19.260 | 1.00 | 0.00 |
| 674 | CG | LEU | E | 224 | −35.049 | −23.631 | −18.577 | 1.00 | 0.00 |
| 675 | CD1 | LEU | E | 224 | −35.125 | −22.142 | −18.963 | 1.00 | 0.00 |
| 676 | CD2 | LEU | E | 224 | −36.351 | −24.343 | −19.000 | 1.00 | 0.00 |
| 677 | N | SER | E | 225 | −32.484 | −26.638 | −20.737 | 1.00 | 0.00 |
| 678 | CA | SER | E | 225 | −31.278 | −27.130 | −21.379 | 1.00 | 0.00 |
| 679 | C | SER | E | 225 | −30.385 | −25.958 | −21.679 | 1.00 | 0.00 |
| 680 | O | SER | E | 225 | −29.295 | −25.875 | −21.134 | 1.00 | 0.00 |
| 681 | CB | SER | E | 225 | −31.635 | −27.885 | −22.674 | 1.00 | 0.00 |
| 682 | OG | SER | E | 225 | −30.462 | −28.513 | −23.199 | 1.00 | 0.00 |
| 683 | N | LEU | E | 226 | −30.865 | −25.044 | −22.545 | 1.00 | 0.00 |
| 684 | CA | LEU | E | 226 | −30.085 | −23.846 | −22.782 | 1.00 | 0.00 |
| 685 | C | LEU | E | 226 | −30.339 | −22.872 | −21.665 | 1.00 | 0.00 |
| 686 | O | LEU | E | 226 | −31.452 | −22.783 | −21.169 | 1.00 | 0.00 |
| 687 | CB | LEU | E | 226 | −30.446 | −23.209 | −24.138 | 1.00 | 0.00 |
| 688 | CG | LEU | E | 226 | −29.515 | −22.012 | −24.429 | 1.00 | 0.00 |
| 689 | CD1 | LEU | E | 226 | −28.072 | −22.508 | −24.635 | 1.00 | 0.00 |
| 690 | CD2 | LEU | E | 226 | −29.999 | −21.276 | −25.692 | 1.00 | 0.00 |
| 691 | N | VAL | E | 227 | −29.270 | −22.150 | −21.273 | 1.00 | 0.00 |
| 692 | CA | VAL | E | 227 | −29.412 | −21.202 | −20.181 | 1.00 | 0.00 |
| 693 | C | VAL | E | 227 | −28.423 | −20.080 | −20.345 | 1.00 | 0.00 |
| 694 | O | VAL | E | 227 | −27.425 | −20.251 | −21.025 | 1.00 | 0.00 |
| 695 | CB | VAL | E | 227 | −29.190 | −21.910 | −18.827 | 1.00 | 0.00 |
| 696 | CG1 | VAL | E | 227 | −30.347 | −22.879 | −18.505 | 1.00 | 0.00 |
| 697 | CG2 | VAL | E | 227 | −27.847 | −22.671 | −18.837 | 1.00 | 0.00 |
| 698 | N | THR | E | 228 | −28.693 | −18.912 | −19.730 | 1.00 | 0.00 |
| 699 | CA | THR | E | 228 | −27.735 | −17.828 | −19.867 | 1.00 | 0.00 |
| 700 | C | THR | E | 228 | −26.761 | −17.841 | −18.723 | 1.00 | 0.00 |
| 701 | O | THR | E | 228 | −27.171 | −17.836 | −17.574 | 1.00 | 0.00 |
| 702 | CB | THR | E | 228 | −28.446 | −16.466 | −19.973 | 1.00 | 0.00 |
| 703 | OG1 | THR | E | 228 | −29.341 | −16.477 | −21.090 | 1.00 | 0.00 |
| 704 | CG2 | THR | E | 228 | −27.395 | −15.360 | −20.184 | 1.00 | 0.00 |
| 705 | N | LEU | E | 229 | −25.456 | −17.856 | −19.057 | 1.00 | 0.00 |
| 706 | CA | LEU | E | 229 | −24.453 | −17.806 | −18.007 | 1.00 | 0.00 |
| 707 | C | LEU | E | 229 | −24.330 | −16.371 | −17.589 | 1.00 | 0.00 |
| 708 | O | LEU | E | 229 | −24.545 | −16.056 | −16.430 | 1.00 | 0.00 |
| 709 | CB | LEU | E | 229 | −23.112 | −18.313 | −18.571 | 1.00 | 0.00 |
| 710 | CG | LEU | E | 229 | −23.262 | −19.769 | −19.052 | 1.00 | 0.00 |
| 711 | CD1 | LEU | E | 229 | −21.961 | −20.209 | −19.752 | 1.00 | 0.00 |
| 712 | CD2 | LEU | E | 229 | −23.563 | −20.692 | −17.853 | 1.00 | 0.00 |
| 713 | N | PHE | E | 230 | −24.006 | −15.492 | −18.556 | 1.00 | 0.00 |
| 714 | CA | PHE | E | 230 | −24.001 | −14.076 | −18.233 | 1.00 | 0.00 |
| 715 | C | PHE | E | 230 | −24.348 | −13.257 | −19.443 | 1.00 | 0.00 |
| 716 | O | PHE | E | 230 | −24.434 | −13.799 | −20.533 | 1.00 | 0.00 |
| 717 | CB | PHE | E | 230 | −22.671 | −13.624 | −17.596 | 1.00 | 0.00 |
| 718 | CG | PHE | E | 230 | −21.462 | −13.955 | −18.473 | 1.00 | 0.00 |
| 719 | CD1 | PHE | E | 230 | −20.930 | −12.997 | −19.341 | 1.00 | 0.00 |
| 720 | CD2 | PHE | E | 230 | −20.874 | −15.222 | −18.401 | 1.00 | 0.00 |
| 721 | CE1 | PHE | E | 230 | −19.800 | −13.293 | −20.110 | 1.00 | 0.00 |
| 722 | CE2 | PHE | E | 230 | −19.746 | −15.523 | −19.169 | 1.00 | 0.00 |
| 723 | CZ | PHE | E | 230 | −19.195 | −14.548 | −20.005 | 1.00 | 0.00 |
| 724 | N | ARG | E | 231 | −24.561 | −11.941 | −19.258 | 1.00 | 0.00 |
| 725 | CA | ARG | E | 231 | −25.031 | −11.151 | −20.384 | 1.00 | 0.00 |
| 726 | C | ARG | E | 231 | −24.566 | −9.727 | −20.277 | 1.00 | 0.00 |
| 727 | O | ARG | E | 231 | −23.881 | −9.381 | −19.328 | 1.00 | 0.00 |
| 728 | CB | ARG | E | 231 | −26.572 | −11.187 | −20.399 | 1.00 | 0.00 |
| 729 | CG | ARG | E | 231 | −27.136 | −10.812 | −19.012 | 1.00 | 0.00 |
| 730 | CD | ARG | E | 231 | −28.675 | −10.753 | −19.074 | 1.00 | 0.00 |
| 731 | NE | ARG | E | 231 | −29.202 | −10.266 | −17.810 | 1.00 | 0.00 |
| 732 | CZ | ARG | E | 231 | −29.755 | −9.091 | −17.712 | 1.00 | 0.00 |
| 733 | NH1 | ARG | E | 231 | −29.890 | −8.320 | −18.751 | 1.00 | 0.00 |
| 734 | NH2 | ARG | E | 231 | −30.175 | −8.673 | −16.555 | 1.00 | 0.00 |
| 735 | N | CYS | E | 232 | −24.953 | −8.908 | −21.276 | 1.00 | 0.00 |
| 736 | CA | CYS | E | 232 | −24.508 | −7.526 | −21.275 | 1.00 | 0.00 |
| 737 | C | CYS | E | 232 | −25.519 | −6.656 | −21.963 | 1.00 | 0.00 |
| 738 | O | CYS | E | 232 | −26.326 | −7.148 | −22.738 | 1.00 | 0.00 |
| 739 | CB | CYS | E | 232 | −23.123 | −7.382 | −21.934 | 1.00 | 0.00 |
| 740 | SG | CYS | E | 232 | −22.046 | −6.567 | −20.720 | 1.00 | 0.00 |
| 741 | N | ILE | E | 233 | −25.478 | −5.343 | −21.663 | 1.00 | 0.00 |
| 742 | CA | ILE | E | 233 | −26.447 | −4.457 | −22.285 | 1.00 | 0.00 |
| 743 | C | ILE | E | 233 | −26.019 | −3.017 | −22.248 | 1.00 | 0.00 |
| 744 | O | ILE | E | 233 | −25.223 | −2.623 | −21.411 | 1.00 | 0.00 |
| 745 | CB | ILE | E | 233 | −27.856 | −4.651 | −21.685 | 1.00 | 0.00 |
| 746 | CG1 | ILE | E | 233 | −28.910 | −3.921 | −22.540 | 1.00 | 0.00 |
| 747 | CG2 | ILE | E | 233 | −27.894 | −4.133 | −20.233 | 1.00 | 0.00 |
| 748 | CD1 | ILE | E | 233 | −30.321 | −4.406 | −22.159 | 1.00 | 0.00 |
| 749 | N | GLN | E | 234 | −26.577 | −2.237 | −23.194 | 1.00 | 0.00 |
| 750 | CA | GLN | E | 234 | −26.233 | −0.827 | −23.266 | 1.00 | 0.00 |
| 751 | C | GLN | E | 234 | −27.329 | −0.101 | −23.991 | 1.00 | 0.00 |
| 752 | O | GLN | E | 234 | −27.687 | −0.514 | −25.082 | 1.00 | 0.00 |
| 753 | CB | GLN | E | 234 | −24.967 | −0.658 | −24.130 | 1.00 | 0.00 |
| 754 | CG | GLN | E | 234 | −23.720 | −1.224 | −23.427 | 1.00 | 0.00 |
| 755 | CD | GLN | E | 234 | −23.192 | −0.203 | −22.454 | 1.00 | 0.00 |
| 756 | OE1 | GLN | E | 234 | −22.152 | 0.386 | −22.703 | 1.00 | 0.00 |
| 757 | NE2 | GLN | E | 234 | −23.909 | 0.004 | −21.332 | 1.00 | 0.00 |
| 758 | N | ASN | E | 235 | −27.861 | 0.985 | −23.393 | 1.00 | 0.00 |
| 759 | CA | ASN | E | 235 | −28.902 | 1.728 | −24.089 | 1.00 | 0.00 |
| 760 | C | ASN | E | 235 | −28.322 | 2.339 | −25.332 | 1.00 | 0.00 |
| 761 | O | ASN | E | 235 | −27.120 | 2.554 | −25.376 | 1.00 | 0.00 |
| 762 | CB | ASN | E | 235 | −29.535 | 2.825 | −23.205 | 1.00 | 0.00 |
| 763 | CG | ASN | E | 235 | −30.013 | 2.240 | −21.903 | 1.00 | 0.00 |
| 764 | OD1 | ASN | E | 235 | −31.133 | 1.761 | −21.822 | 1.00 | 0.00 |
| 765 | ND2 | ASN | E | 235 | −29.158 | 2.285 | −20.864 | 1.00 | 0.00 |
| 766 | N | MET | E | 236 | −29.166 | 2.601 | −26.350 | 1.00 | 0.00 |
| 767 | CA | MET | E | 236 | −28.623 | 3.173 | −27.571 | 1.00 | 0.00 |
| 768 | C | MET | E | 236 | −29.089 | 4.585 | −27.804 | 1.00 | 0.00 |
| 769 | O | MET | E | 236 | −30.208 | 4.912 | −27.443 | 1.00 | 0.00 |
| 770 | CB | MET | E | 236 | −28.859 | 2.261 | −28.790 | 1.00 | 0.00 |
| 771 | CG | MET | E | 236 | −28.235 | 0.874 | −28.530 | 1.00 | 0.00 |
| 772 | SD | MET | E | 236 | −26.436 | 1.014 | −28.282 | 1.00 | 0.00 |
| 773 | CE | MET | E | 236 | −25.967 | 1.404 | −29.993 | 1.00 | 0.00 |
| 774 | N | PRO | E | 237 | −28.225 | 5.440 | −28.393 | 1.00 | 0.00 |
| 775 | CA | PRO | E | 237 | −28.590 | 6.825 | −28.603 | 1.00 | 0.00 |
| 776 | C | PRO | E | 237 | −29.550 | 6.954 | −29.749 | 1.00 | 0.00 |
| 777 | O | PRO | E | 237 | −29.668 | 6.046 | −30.556 | 1.00 | 0.00 |
| 778 | CB | PRO | E | 237 | −27.242 | 7.454 | −29.012 | 1.00 | 0.00 |
| 779 | CG | PRO | E | 237 | −26.282 | 6.293 | −29.355 | 1.00 | 0.00 |
| 780 | CD | PRO | E | 237 | −26.910 | 4.996 | −28.806 | 1.00 | 0.00 |
| 781 | N | GLU | E | 238 | −30.242 | 8.107 | −29.810 | 1.00 | 0.00 |
| 782 | CA | GLU | E | 238 | −31.158 | 8.325 | −30.914 | 1.00 | 0.00 |
| 783 | C | GLU | E | 238 | −30.391 | 8.447 | −32.199 | 1.00 | 0.00 |
| 784 | O | GLU | E | 238 | −30.904 | 8.048 | −33.232 | 1.00 | 0.00 |
| 785 | CB | GLU | E | 238 | −31.928 | 9.637 | −30.677 | 1.00 | 0.00 |
| 786 | CG | GLU | E | 238 | −32.943 | 9.451 | −29.534 | 1.00 | 0.00 |
| 787 | CD | GLU | E | 238 | −33.915 | 8.353 | −29.859 | 1.00 | 0.00 |
| 788 | OE1 | GLU | E | 238 | −34.654 | 8.490 | −30.871 | 1.00 | 0.00 |
| 789 | OE2 | GLU | E | 238 | −33.942 | 7.349 | −29.099 | 1.00 | 0.00 |
| 790 | N | THR | E | 239 | −29.159 | 8.989 | −32.135 | 1.00 | 0.00 |
| 791 | CA | THR | E | 239 | −28.390 | 9.109 | −33.360 | 1.00 | 0.00 |
| 792 | C | THR | E | 239 | −27.008 | 8.547 | −33.189 | 1.00 | 0.00 |
| 793 | O | THR | E | 239 | −26.466 | 8.567 | −32.095 | 1.00 | 0.00 |
| 794 | CB | THR | E | 239 | −28.315 | 10.571 | −33.840 | 1.00 | 0.00 |
| 795 | OG1 | THR | E | 239 | −27.721 | 11.383 | −32.820 | 1.00 | 0.00 |
| 796 | CG2 | THR | E | 239 | −29.730 | 11.094 | −34.132 | 1.00 | 0.00 |
| 797 | N | LEU | E | 240 | −26.451 | 8.038 | −34.305 | 1.00 | 0.00 |
| 798 | CA | LEU | E | 240 | −25.097 | 7.510 | −34.275 | 1.00 | 0.00 |
| 799 | C | LEU | E | 240 | −24.898 | 6.496 | −33.174 | 1.00 | 0.00 |
| 800 | O | LEU | E | 240 | −23.986 | 6.664 | −32.379 | 1.00 | 0.00 |
| 801 | CB | LEU | E | 240 | −24.093 | 8.679 | −34.199 | 1.00 | 0.00 |
| 802 | CG | LEU | E | 240 | −24.358 | 9.673 | −35.347 | 1.00 | 0.00 |
| 803 | CD1 | LEU | E | 240 | −23.549 | 10.962 | −35.115 | 1.00 | 0.00 |
| 804 | CD2 | LEU | E | 240 | −23.961 | 9.040 | −36.696 | 1.00 | 0.00 |
| 805 | N | PRO | E | 241 | −25.736 | 5.436 | −33.103 | 1.00 | 0.00 |
| 806 | CA | PRO | E | 241 | −25.576 | 4.443 | −32.062 | 1.00 | 0.00 |
| 807 | C | PRO | E | 241 | −24.302 | 3.682 | −32.284 | 1.00 | 0.00 |
| 808 | O | PRO | E | 241 | −24.062 | 3.169 | −33.366 | 1.00 | 0.00 |
| 809 | CB | PRO | E | 241 | −26.792 | 3.521 | −32.276 | 1.00 | 0.00 |
| 810 | CG | PRO | E | 241 | −27.582 | 4.066 | −33.487 | 1.00 | 0.00 |
| 811 | CD | PRO | E | 241 | −26.805 | 5.266 | −34.063 | 1.00 | 0.00 |
| 812 | N | ASN | E | 242 | −23.469 | 3.632 | −31.228 | 1.00 | 0.00 |
| 813 | CA | ASN | E | 242 | −22.172 | 3.006 | −31.395 | 1.00 | 0.00 |
| 814 | C | ASN | E | 242 | −21.644 | 2.618 | −30.046 | 1.00 | 0.00 |
| 815 | O | ASN | E | 242 | −20.808 | 3.308 | −29.485 | 1.00 | 0.00 |
| 816 | CB | ASN | E | 242 | −21.236 | 3.991 | −32.125 | 1.00 | 0.00 |
| 817 | CG | ASN | E | 242 | −20.431 | 3.272 | −33.174 | 1.00 | 0.00 |
| 818 | OD1 | ASN | E | 242 | −19.245 | 3.523 | −33.311 | 1.00 | 0.00 |
| 819 | ND2 | ASN | E | 242 | −21.075 | 2.364 | −33.932 | 1.00 | 0.00 |
| 820 | N | ASN | E | 243 | −22.154 | 1.482 | −29.531 | 1.00 | 0.00 |
| 821 | CA | ASN | E | 243 | −21.739 | 1.059 | −28.206 | 1.00 | 0.00 |
| 822 | C | ASN | E | 243 | −21.292 | −0.375 | −28.206 | 1.00 | 0.00 |
| 823 | O | ASN | E | 243 | −22.032 | −1.251 | −28.628 | 1.00 | 0.00 |
| 824 | CB | ASN | E | 243 | −22.945 | 1.206 | −27.261 | 1.00 | 0.00 |
| 825 | CG | ASN | E | 243 | −23.064 | 2.611 | −26.743 | 1.00 | 0.00 |
| 826 | OD1 | ASN | E | 243 | −22.084 | 3.334 | −26.656 | 1.00 | 0.00 |
| 827 | ND2 | ASN | E | 243 | −24.301 | 3.007 | −26.393 | 1.00 | 0.00 |
| 828 | N | SER | E | 244 | −20.057 | −0.605 | −27.717 | 1.00 | 0.00 |
| 829 | CA | SER | E | 244 | −19.590 | −1.975 | −27.605 | 1.00 | 0.00 |
| 830 | C | SER | E | 244 | −20.173 | −2.566 | −26.352 | 1.00 | 0.00 |
| 831 | O | SER | E | 244 | −20.776 | −1.838 | −25.579 | 1.00 | 0.00 |
| 832 | CB | SER | E | 244 | −18.049 | −2.028 | −27.574 | 1.00 | 0.00 |
| 833 | OG | SER | E | 244 | −17.532 | −1.140 | −26.577 | 1.00 | 0.00 |
| 834 | N | CYS | E | 245 | −19.996 | −3.890 | −26.166 | 1.00 | 0.00 |
| 835 | CA | CYS | E | 245 | −20.591 | −4.531 | −25.007 | 1.00 | 0.00 |
| 836 | C | CYS | E | 245 | −19.752 | −5.719 | −24.620 | 1.00 | 0.00 |
| 837 | O | CYS | E | 245 | −19.854 | −6.779 | −25.219 | 1.00 | 0.00 |
| 838 | CB | CYS | E | 245 | −22.041 | −4.939 | −25.332 | 1.00 | 0.00 |
| 839 | SG | CYS | E | 245 | −23.033 | −4.653 | −23.835 | 1.00 | 0.00 |
| 840 | N | TYR | E | 246 | −18.904 | −5.526 | −23.592 | 1.00 | 0.00 |
| 841 | CA | TYR | E | 246 | −18.047 | −6.619 | −23.172 | 1.00 | 0.00 |
| 842 | C | TYR | E | 246 | −18.519 | −7.223 | −21.879 | 1.00 | 0.00 |
| 843 | O | TYR | E | 246 | −18.997 | −6.519 | −21.004 | 1.00 | 0.00 |
| 844 | CB | TYR | E | 246 | −16.598 | −6.110 | −23.044 | 1.00 | 0.00 |
| 845 | CG | TYR | E | 246 | −15.684 | −7.213 | −22.522 | 1.00 | 0.00 |
| 846 | CD1 | TYR | E | 246 | −15.493 | −7.374 | −21.146 | 1.00 | 0.00 |
| 847 | CD2 | TYR | E | 246 | −15.043 | −8.063 | −23.427 | 1.00 | 0.00 |
| 848 | CE1 | TYR | E | 246 | −14.679 | −8.407 | −20.674 | 1.00 | 0.00 |
| 849 | CE2 | TYR | E | 246 | −14.220 | −9.087 | −22.952 | 1.00 | 0.00 |
| 850 | CZ | TYR | E | 246 | −14.042 | −9.263 | −21.578 | 1.00 | 0.00 |
| 851 | OH | TYR | E | 246 | −13.230 | −10.289 | −21.109 | 1.00 | 0.00 |
| 852 | N | SER | E | 247 | −18.365 | −8.557 | −21.774 | 1.00 | 0.00 |
| 853 | CA | SER | E | 247 | −18.715 | −9.215 | −20.529 | 1.00 | 0.00 |
| 854 | C | SER | E | 247 | −17.935 | −10.496 | −20.407 | 1.00 | 0.00 |
| 855 | O | SER | E | 247 | −17.451 | −11.014 | −21.402 | 1.00 | 0.00 |
| 856 | CB | SER | E | 247 | −20.229 | −9.491 | −20.452 | 1.00 | 0.00 |
| 857 | OG | SER | E | 247 | −20.581 | −9.902 | −19.127 | 1.00 | 0.00 |
| 858 | N | ALA | E | 248 | −17.805 | −11.002 | −19.166 | 1.00 | 0.00 |
| 859 | CA | ALA | E | 248 | −17.025 | −12.213 | −18.977 | 1.00 | 0.00 |
| 860 | C | ALA | E | 248 | −17.304 | −12.827 | −17.632 | 1.00 | 0.00 |
| 861 | O | ALA | E | 248 | −17.952 | −12.211 | −16.800 | 1.00 | 0.00 |
| 862 | CB | ALA | E | 248 | −15.524 | −11.900 | −19.117 | 1.00 | 0.00 |
| 863 | N | GLY | E | 249 | −16.806 | −14.062 | −17.429 | 1.00 | 0.00 |
| 864 | CA | GLY | E | 249 | −17.040 | −14.710 | −16.154 | 1.00 | 0.00 |
| 865 | C | GLY | E | 249 | −16.499 | −16.113 | −16.157 | 1.00 | 0.00 |
| 866 | O | GLY | E | 249 | −16.216 | −16.663 | −17.209 | 1.00 | 0.00 |
| 867 | N | ILE | E | 250 | −16.360 | −16.694 | −14.950 | 1.00 | 0.00 |
| 868 | CA | ILE | E | 250 | −15.874 | −18.061 | −14.881 | 1.00 | 0.00 |
| 869 | C | ILE | E | 250 | −17.019 | −19.024 | −14.719 | 1.00 | 0.00 |
| 870 | O | ILE | E | 250 | −18.068 | −18.655 | −14.215 | 1.00 | 0.00 |
| 871 | CB | ILE | E | 250 | −14.874 | −18.221 | −13.719 | 1.00 | 0.00 |
| 872 | CG1 | ILE | E | 250 | −13.755 | −17.167 | −13.840 | 1.00 | 0.00 |
| 873 | CG2 | ILE | E | 250 | −14.260 | −19.637 | −13.738 | 1.00 | 0.00 |
| 874 | CD1 | ILE | E | 250 | −12.881 | −17.177 | −12.571 | 1.00 | 0.00 |
| 875 | N | ALA | E | 251 | −16.799 | −20.277 | −15.162 | 1.00 | 0.00 |
| 876 | CA | ALA | E | 251 | −17.842 | −21.273 | −15.000 | 1.00 | 0.00 |
| 877 | C | ALA | E | 251 | −17.295 | −22.655 | −15.223 | 1.00 | 0.00 |
| 878 | O | ALA | E | 251 | −16.449 | −22.853 | −16.080 | 1.00 | 0.00 |
| 879 | CB | ALA | E | 251 | −19.013 | −21.014 | −15.964 | 1.00 | 0.00 |
| 880 | N | LYS | E | 252 | −17.793 | −23.618 | −14.425 | 1.00 | 0.00 |
| 881 | CA | LYS | E | 252 | −17.327 | −24.980 | −14.589 | 1.00 | 0.00 |
| 882 | C | LYS | E | 252 | −18.193 | −25.662 | −15.610 | 1.00 | 0.00 |
| 883 | O | LYS | E | 252 | −19.368 | −25.883 | −15.360 | 1.00 | 0.00 |
| 884 | CB | LYS | E | 252 | −17.424 | −25.692 | −13.227 | 1.00 | 0.00 |
| 885 | CG | LYS | E | 252 | −16.719 | −27.060 | −13.301 | 1.00 | 0.00 |
| 886 | CD | LYS | E | 252 | −16.938 | −27.811 | −11.978 | 1.00 | 0.00 |
| 887 | CE | LYS | E | 252 | −16.399 | −29.248 | −12.106 | 1.00 | 0.00 |
| 888 | NZ | LYS | E | 252 | −16.782 | −30.013 | −10.909 | 1.00 | 0.00 |
| 889 | N | LEU | E | 253 | −17.597 | −25.990 | −16.771 | 1.00 | 0.00 |
| 890 | CA | LEU | E | 253 | −18.383 | −26.645 | −17.801 | 1.00 | 0.00 |
| 891 | C | LEU | E | 253 | −18.089 | −28.117 | −17.863 | 1.00 | 0.00 |
| 892 | O | LEU | E | 253 | −17.008 | −28.544 | −17.490 | 1.00 | 0.00 |
| 893 | CB | LEU | E | 253 | −18.106 | −26.002 | −19.171 | 1.00 | 0.00 |
| 894 | CG | LEU | E | 253 | −18.472 | −24.507 | −19.126 | 1.00 | 0.00 |
| 895 | CD1 | LEU | E | 253 | −18.050 | −23.844 | −20.451 | 1.00 | 0.00 |
| 896 | CD2 | LEU | E | 253 | −19.989 | −24.328 | −18.902 | 1.00 | 0.00 |
| 897 | N | GLU | E | 254 | −19.081 | −28.891 | −18.340 | 1.00 | 0.00 |
| 898 | CA | GLU | E | 254 | −18.873 | −30.323 | −18.471 | 1.00 | 0.00 |
| 899 | C | GLU | E | 254 | −18.609 | −30.673 | −19.906 | 1.00 | 0.00 |
| 900 | O | GLU | E | 254 | −18.878 | −29.877 | −20.791 | 1.00 | 0.00 |
| 901 | CB | GLU | E | 254 | −20.131 | −31.096 | −18.033 | 1.00 | 0.00 |
| 902 | CG | GLU | E | 254 | −20.483 | −30.763 | −16.570 | 1.00 | 0.00 |
| 903 | CD | GLU | E | 254 | −21.436 | −31.799 | −16.048 | 1.00 | 0.00 |
| 904 | OE1 | GLU | E | 254 | −22.548 | −31.931 | −16.627 | 1.00 | 0.00 |
| 905 | OE2 | GLU | E | 254 | −21.072 | −32.485 | −15.056 | 1.00 | 0.00 |
| 906 | N | GLU | E | 255 | −18.083 | −31.893 | −20.126 | 1.00 | 0.00 |
| 907 | CA | GLU | E | 255 | −17.876 | −32.330 | −21.494 | 1.00 | 0.00 |
| 908 | C | GLU | E | 255 | −19.207 | −32.500 | −22.173 | 1.00 | 0.00 |
| 909 | O | GLU | E | 255 | −20.177 | −32.863 | −21.526 | 1.00 | 0.00 |
| 910 | CB | GLU | E | 255 | −17.115 | −33.667 | −21.479 | 1.00 | 0.00 |
| 911 | CG | GLU | E | 255 | −16.627 | −34.020 | −22.897 | 1.00 | 0.00 |
| 912 | CD | GLU | E | 255 | −15.958 | −35.365 | −22.889 | 1.00 | 0.00 |
| 913 | OE1 | GLU | E | 255 | −15.039 | −35.573 | −22.050 | 1.00 | 0.00 |
| 914 | OE2 | GLU | E | 255 | −16.353 | −36.222 | −23.723 | 1.00 | 0.00 |
| 915 | N | GLY | E | 256 | −19.243 | −32.213 | −23.488 | 1.00 | 0.00 |
| 916 | CA | GLY | E | 256 | −20.513 | −32.288 | −24.186 | 1.00 | 0.00 |
| 917 | C | GLY | E | 256 | −21.244 | −30.976 | −24.116 | 1.00 | 0.00 |
| 918 | O | GLY | E | 256 | −22.113 | −30.741 | −24.940 | 1.00 | 0.00 |
| 919 | N | ASP | E | 257 | −20.897 | −30.116 | −23.137 | 1.00 | 0.00 |
| 920 | CA | ASP | E | 257 | −21.572 | −28.830 | −23.055 | 1.00 | 0.00 |
| 921 | C | ASP | E | 257 | −21.231 | −27.959 | −24.231 | 1.00 | 0.00 |
| 922 | O | ASP | E | 257 | −20.271 | −28.226 | −24.937 | 1.00 | 0.00 |
| 923 | CB | ASP | E | 257 | −21.196 | −28.094 | −21.755 | 1.00 | 0.00 |
| 924 | CG | ASP | E | 257 | −21.949 | −28.631 | −20.570 | 1.00 | 0.00 |
| 925 | OD1 | ASP | E | 257 | −23.004 | −29.291 | −20.768 | 1.00 | 0.00 |
| 926 | OD2 | ASP | E | 257 | −21.478 | −28.402 | −19.426 | 1.00 | 0.00 |
| 927 | N | GLU | E | 258 | −22.039 | −26.902 | −24.437 | 1.00 | 0.00 |
| 928 | CA | GLU | E | 258 | −21.756 | −26.005 | −25.543 | 1.00 | 0.00 |
| 929 | C | GLU | E | 258 | −21.968 | −24.578 | −25.129 | 1.00 | 0.00 |
| 930 | O | GLU | E | 258 | −22.597 | −24.315 | −24.116 | 1.00 | 0.00 |
| 931 | CB | GLU | E | 258 | −22.639 | −26.336 | −26.761 | 1.00 | 0.00 |
| 932 | CG | GLU | E | 258 | −22.225 | −27.694 | −27.360 | 1.00 | 0.00 |
| 933 | CD | GLU | E | 258 | −22.835 | −27.849 | −28.723 | 1.00 | 0.00 |
| 934 | OE1 | GLU | E | 258 | −24.092 | −27.834 | −28.816 | 1.00 | 0.00 |
| 935 | OE2 | GLU | E | 258 | −22.059 | −27.988 | −29.706 | 1.00 | 0.00 |
| 936 | N | LEU | E | 259 | −21.419 | −23.652 | −25.937 | 1.00 | 0.00 |
| 937 | CA | LEU | E | 259 | −21.587 | −22.249 | −25.611 | 1.00 | 0.00 |
| 938 | C | LEU | E | 259 | −21.977 | −21.472 | −26.834 | 1.00 | 0.00 |
| 939 | O | LEU | E | 259 | −21.717 | −21.902 | −27.947 | 1.00 | 0.00 |
| 940 | CB | LEU | E | 259 | −20.292 | −21.674 | −25.010 | 1.00 | 0.00 |
| 941 | CG | LEU | E | 259 | −19.949 | −22.393 | −23.691 | 1.00 | 0.00 |
| 942 | CD1 | LEU | E | 259 | −18.583 | −21.888 | −23.190 | 1.00 | 0.00 |
| 943 | CD2 | LEU | E | 259 | −21.024 | −22.099 | −22.625 | 1.00 | 0.00 |
| 944 | N | GLN | E | 260 | −22.622 | −20.312 | −26.611 | 1.00 | 0.00 |
| 945 | CA | GLN | E | 260 | −23.029 | −19.510 | −27.749 | 1.00 | 0.00 |
| 946 | C | GLN | E | 260 | −23.353 | −18.101 | −27.342 | 1.00 | 0.00 |
| 947 | O | GLN | E | 260 | −23.750 | −17.854 | −26.214 | 1.00 | 0.00 |
| 948 | CB | GLN | E | 260 | −24.200 | −20.157 | −28.516 | 1.00 | 0.00 |
| 949 | CG | GLN | E | 260 | −25.466 | −20.182 | −27.636 | 1.00 | 0.00 |
| 950 | CD | GLN | E | 260 | −26.568 | −20.985 | −28.279 | 1.00 | 0.00 |
| 951 | OE1 | GLN | E | 260 | −26.354 | −21.663 | −29.272 | 1.00 | 0.00 |
| 952 | NE2 | GLN | E | 260 | −27.777 | −20.904 | −27.698 | 1.00 | 0.00 |
| 953 | N | LEU | E | 261 | −23.168 | −17.172 | −28.296 | 1.00 | 0.00 |
| 954 | CA | LEU | F | 261 | −23.503 | −15.792 | −28.008 | 1.00 | 0.00 |
| 955 | C | LEU | E | 261 | −24.704 | −15.425 | −28.833 | 1.00 | 0.00 |
| 956 | O | LEU | E | 261 | −24.611 | −15.353 | −30.049 | 1.00 | 0.00 |
| 957 | CB | LEU | E | 261 | −22.285 | −14.916 | −28.362 | 1.00 | 0.00 |
| 958 | CG | LEU | E | 261 | −22.409 | −13.525 | −27.706 | 1.00 | 0.00 |
| 959 | CD1 | LEU | E | 261 | −21.054 | −12.796 | −27.793 | 1.00 | 0.00 |
| 960 | CD2 | LEU | E | 261 | −23.499 | −12.694 | −28.415 | 1.00 | 0.00 |
| 961 | N | ALA | E | 262 | −25.846 | −15.193 | −28.159 | 1.00 | 0.00 |
| 962 | CA | ALA | E | 262 | −27.038 | −14.856 | −28.915 | 1.00 | 0.00 |
| 963 | C | ALA | E | 262 | −27.487 | −13.448 | −28.644 | 1.00 | 0.00 |
| 964 | O | ALA | E | 262 | −27.237 | −12.908 | −27.578 | 1.00 | 0.00 |
| 965 | CB | ALA | E | 262 | −28.170 | −15.834 | −28.554 | 1.00 | 0.00 |
| 966 | N | ILE | E | 263 | −28.170 | −12.855 | −29.641 | 1.00 | 0.00 |
| 967 | CA | ILE | E | 263 | −28.754 | −11.550 | −29.404 | 1.00 | 0.00 |
| 968 | C | ILE | E | 263 | −30.248 | −11.718 | −29.383 | 1.00 | 0.00 |
| 969 | O | ILE | E | 263 | −30.802 | −12.153 | −30.380 | 1.00 | 0.00 |
| 970 | CB | ILE | E | 263 | −28.300 | −10.533 | −30.470 | 1.00 | 0.00 |
| 971 | CG1 | ILE | E | 263 | −26.774 | −10.336 | −30.368 | 1.00 | 0.00 |
| 972 | CG2 | ILE | E | 263 | −29.013 | −9.188 | −30.229 | 1.00 | 0.00 |
| 973 | CD1 | ILE | E | 263 | −26.283 | −9.396 | −31.486 | 1.00 | 0.00 |
| 974 | N | PRO | E | 264 | −30.900 | −11.387 | −28.247 | 1.00 | 0.00 |
| 975 | CA | PRO | E | 264 | −32.336 | −11.544 | −28.149 | 1.00 | 0.00 |
| 976 | C | PRO | E | 264 | −33.031 | −10.481 | −28.955 | 1.00 | 0.00 |
| 977 | O | PRO | E | 264 | −33.638 | −9.573 | −28.409 | 1.00 | 0.00 |
| 978 | CB | PRO | E | 264 | −32.572 | −11.363 | −26.636 | 1.00 | 0.00 |
| 979 | CG | PRO | E | 264 | −31.266 | −10.818 | −26.018 | 1.00 | 0.00 |
| 980 | CD | PRO | E | 264 | −30.168 | −10.894 | −27.099 | 1.00 | 0.00 |
| 981 | N | ARG | E | 265 | −32.934 | −10.619 | −30.291 | 1.00 | 0.00 |
| 982 | CA | ARG | E | 265 | −33.590 | −9.666 | −31.163 | 1.00 | 0.00 |
| 983 | C | ARG | E | 265 | −33.524 | −10.164 | −32.578 | 1.00 | 0.00 |
| 984 | O | ARG | E | 265 | −32.623 | −10.913 | −32.922 | 1.00 | 0.00 |
| 985 | CB | ARG | E | 265 | −32.917 | −8.284 | −31.059 | 1.00 | 0.00 |
| 986 | CG | ARG | E | 265 | −33.743 | −7.234 | −31.828 | 1.00 | 0.00 |
| 987 | CD | ARG | E | 265 | −33.346 | −5.815 | −31.378 | 1.00 | 0.00 |
| 988 | NE | ARG | E | 265 | −34.150 | −4.841 | −32.094 | 1.00 | 0.00 |
| 989 | CZ | ARG | E | 265 | −35.245 | −4.355 | −31.581 | 1.00 | 0.00 |
| 990 | NH1 | ARG | E | 265 | −35.665 | −4.723 | −30.407 | 1.00 | 0.00 |
| 991 | NH2 | ARG | E | 265 | −35.934 | −3.484 | −32.257 | 1.00 | 0.00 |
| 992 | N | GLU | E | 266 | −34.500 | −9.745 | −33.405 | 1.00 | 0.00 |
| 993 | CA | GLU | E | 266 | −34.472 | −10.187 | −34.785 | 1.00 | 0.00 |
| 994 | C | GLU | E | 266 | −33.782 | −9.156 | −35.626 | 1.00 | 0.00 |
| 995 | O | GLU | E | 266 | −34.137 | −7.989 | −35.573 | 1.00 | 0.00 |
| 996 | CB | GLU | E | 266 | −35.903 | −10.422 | −35.296 | 1.00 | 0.00 |
| 997 | CG | GLU | E | 266 | −36.494 | −11.672 | −34.616 | 1.00 | 0.00 |
| 998 | CD | GLU | E | 266 | −37.958 | −11.775 | −34.933 | 1.00 | 0.00 |
| 999 | OE1 | GLU | E | 266 | −38.293 | −12.024 | −36.122 | 1.00 | 0.00 |
| 1000 | OE2 | GLU | E | 266 | −38.778 | −11.613 | −33.991 | 1.00 | 0.00 |
| 1001 | N | ASN | E | 267 | −32.780 | −9.613 | −36.404 | 1.00 | 0.00 |
| 1002 | CA | ASN | E | 267 | −32.064 | −8.704 | −37.284 | 1.00 | 0.00 |
| 1003 | C | ASN | E | 267 | −31.417 | −7.599 | −36.495 | 1.00 | 0.00 |
| 1004 | O | ASN | E | 267 | −31.638 | −6.431 | −36.775 | 1.00 | 0.00 |
| 1005 | CB | ASN | E | 267 | −32.980 | −8.175 | −38.407 | 1.00 | 0.00 |
| 1006 | CG | ASN | E | 267 | −33.761 | −9.306 | −39.015 | 1.00 | 0.00 |
| 1007 | OD1 | ASN | E | 267 | −33.186 | −10.162 | −39.668 | 1.00 | 0.00 |
| 1008 | ND2 | ASN | E | 267 | −35.088 | −9.314 | −38.800 | 1.00 | 0.00 |
| 1009 | N | ALA | E | 268 | −30.608 | −7.988 | −35.488 | 1.00 | 0.00 |
| 1010 | CA | ALA | E | 268 | −29.961 | −6.980 | −34.664 | 1.00 | 0.00 |
| 1011 | C | ALA | E | 268 | −28.924 | −6.222 | −35.445 | 1.00 | 0.00 |
| 1012 | O | ALA | E | 268 | −28.296 | −6.784 | −36.329 | 1.00 | 0.00 |
| 1013 | CB | ALA | E | 268 | −29.324 | −7.624 | −33.417 | 1.00 | 0.00 |
| 1014 | N | GLN | E | 269 | −28.760 | −4.925 | −35.120 | 1.00 | 0.00 |
| 1015 | CA | GLN | E | 269 | −27.779 | −4.140 | −35.848 | 1.00 | 0.00 |
| 1016 | C | GLN | E | 269 | −26.488 | −4.106 | −35.087 | 1.00 | 0.00 |
| 1017 | O | GLN | E | 269 | −26.427 | −3.524 | −34.016 | 1.00 | 0.00 |
| 1018 | CB | GLN | E | 269 | −28.309 | −2.715 | −36.101 | 1.00 | 0.00 |
| 1019 | CG | GLN | E | 269 | −29.582 | −2.754 | −36.970 | 1.00 | 0.00 |
| 1020 | CD | GLN | E | 269 | −29.332 | −3.451 | −38.283 | 1.00 | 0.00 |
| 1021 | OE1 | GLN | E | 269 | −29.187 | −2.793 | −39.300 | 1.00 | 0.00 |
| 1022 | NE2 | GLN | E | 269 | −29.294 | −4.797 | −38.271 | 1.00 | 0.00 |
| 1023 | N | ILE | E | 270 | −25.451 | −4.746 | −35.661 | 1.00 | 0.00 |
| 1024 | CA | ILE | E | 270 | −24.169 | −4.785 | −34.977 | 1.00 | 0.00 |
| 1025 | C | ILE | E | 270 | −23.032 | −4.762 | −35.963 | 1.00 | 0.00 |
| 1026 | O | ILE | E | 270 | −23.238 | −4.906 | −37.158 | 1.00 | 0.00 |
| 1027 | CB | ILE | E | 270 | −24.049 | −6.049 | −34.096 | 1.00 | 0.00 |
| 1028 | CG1 | ILE | E | 270 | −24.281 | −7.308 | −34.954 | 1.00 | 0.00 |
| 1029 | CG2 | ILE | E | 270 | −25.076 | −6.013 | −32.948 | 1.00 | 0.00 |
| 1030 | CD1 | ILE | E | 270 | −23.907 | −8.573 | −34.158 | 1.00 | 0.00 |
| 1031 | N | SER | E | 271 | −21.807 | −4.579 | −35.434 | 1.00 | 0.00 |
| 1032 | CA | SER | E | 271 | −20.649 | −4.617 | −36.305 | 1.00 | 0.00 |
| 1033 | C | SER | E | 271 | −20.134 | −6.026 | −36.408 | 1.00 | 0.00 |
| 1034 | O | SER | E | 271 | −20.186 | −6.764 | −35.437 | 1.00 | 0.00 |
| 1035 | CB | SER | E | 271 | −19.550 | −3.699 | −35.738 | 1.00 | 0.00 |
| 1036 | OG | SER | E | 271 | −18.401 | −3.735 | −36.587 | 1.00 | 0.00 |
| 1037 | N | LEU | E | 272 | −19.631 | −6.399 | −37.600 | 1.00 | 0.00 |
| 1038 | CA | LEU | E | 272 | −19.020 | −7.711 | −37.713 | 1.00 | 0.00 |
| 1039 | C | LEU | E | 272 | −17.524 | −7.576 | −37.676 | 1.00 | 0.00 |
| 1040 | O | LEU | E | 272 | −16.819 | −8.331 | −38.328 | 1.00 | 0.00 |
| 1041 | CB | LEU | E | 272 | −19.505 | −8.466 | −38.965 | 1.00 | 0.00 |
| 1042 | CG | LEU | E | 272 | −20.954 | −8.949 | −38.758 | 1.00 | 0.00 |
| 1043 | CD1 | LEU | E | 272 | −21.445 | −9.624 | −40.051 | 1.00 | 0.00 |
| 1044 | CD2 | LEU | E | 272 | −21.015 | −9.960 | −37.594 | 1.00 | 0.00 |
| 1045 | N | ASP | E | 273 | −17.041 | −6.587 | −36.898 | 1.00 | 0.00 |
| 1046 | CA | ASP | E | 273 | −15.605 | −6.411 | −36.797 | 1.00 | 0.00 |
| 1047 | C | ASP | E | 273 | −15.024 | −7.453 | −35.879 | 1.00 | 0.00 |
| 1048 | O | ASP | E | 273 | −15.704 | −7.913 | −34.974 | 1.00 | 0.00 |
| 1049 | CB | ASP | E | 273 | −15.253 | −5.002 | −36.293 | 1.00 | 0.00 |
| 1050 | CG | ASP | E | 273 | −14.040 | −4.490 | −37.013 | 1.00 | 0.00 |
| 1051 | OD1 | ASP | E | 273 | −12.929 | −5.034 | −36.771 | 1.00 | 0.00 |
| 1052 | OD2 | ASP | E | 273 | −14.193 | −3.534 | −37.820 | 1.00 | 0.00 |
| 1053 | N | GLY | E | 274 | −13.755 | −7.832 | −36.131 | 1.00 | 0.00 |
| 1054 | CA | GLY | E | 274 | −13.148 | −8.849 | −35.294 | 1.00 | 0.00 |
| 1055 | C | GLY | E | 274 | −12.684 | −8.245 | −33.998 | 1.00 | 0.00 |
| 1056 | O | GLY | E | 274 | −12.970 | −8.780 | −32.938 | 1.00 | 0.00 |
| 1057 | N | ASP | E | 275 | −11.959 | −7.112 | −34.098 | 1.00 | 0.00 |
| 1058 | CA | ASP | E | 275 | −11.448 | −6.489 | −32.887 | 1.00 | 0.00 |
| 1059 | C | ASP | E | 275 | −12.549 | −6.034 | −31.970 | 1.00 | 0.00 |
| 1060 | O | ASP | E | 275 | −12.324 | −5.960 | −30.772 | 1.00 | 0.00 |
| 1061 | CB | ASP | E | 275 | −10.554 | −5.292 | −33.260 | 1.00 | 0.00 |
| 1062 | CG | ASP | E | 275 | −11.171 | −4.394 | −34.296 | 1.00 | 0.00 |
| 1063 | OD1 | ASP | E | 275 | −12.373 | −4.037 | −34.161 | 1.00 | 0.00 |
| 1064 | OD2 | ASP | E | 275 | −10.444 | −4.046 | −35.263 | 1.00 | 0.00 |
| 1065 | N | VAL | E | 276 | −13.740 | −5.733 | −32.528 | 1.00 | 0.00 |
| 1066 | CA | VAL | E | 276 | −14.821 | −5.284 | −31.668 | 1.00 | 0.00 |
| 1067 | C | VAL | E | 276 | −15.721 | −6.427 | −31.283 | 1.00 | 0.00 |
| 1068 | O | VAL | E | 276 | −16.030 | −6.586 | −30.112 | 1.00 | 0.00 |
| 1069 | CB | VAL | E | 276 | −15.580 | −4.134 | −32.364 | 1.00 | 0.00 |
| 1070 | CG1 | VAL | E | 276 | −16.442 | −4.637 | −33.541 | 1.00 | 0.00 |
| 1071 | CG2 | VAL | E | 276 | −16.463 | −3.405 | −31.336 | 1.00 | 0.00 |
| 1072 | N | THR | E | 277 | −16.143 | −7.230 | −32.279 | 1.00 | 0.00 |
| 1073 | CA | THR | E | 277 | −17.047 | −8.322 | −31.963 | 1.00 | 0.00 |
| 1074 | C | THR | E | 277 | −16.302 | −9.626 | −31.942 | 1.00 | 0.00 |
| 1075 | O | THR | E | 277 | −15.700 | −10.005 | −32.934 | 1.00 | 0.00 |
| 1076 | CB | THR | E | 277 | −18.212 | −8.348 | −32.971 | 1.00 | 0.00 |
| 1077 | OG1 | THR | E | 277 | −18.863 | −7.075 | −32.956 | 1.00 | 0.00 |
| 1078 | CG2 | THR | E | 277 | −19.233 | −9.425 | −32.559 | 1.00 | 0.00 |
| 1079 | N | PHE | E | 278 | −16.354 | −10.310 | −30.783 | 1.00 | 0.00 |
| 1080 | CA | PHE | E | 278 | −15.628 | −11.563 | −30.666 | 1.00 | 0.00 |
| 1081 | C | PHE | E | 278 | −16.191 | −12.406 | −29.555 | 1.00 | 0.00 |
| 1082 | O | PHE | E | 278 | −17.050 | −11.951 | −28.815 | 1.00 | 0.00 |
| 1083 | CB | PHE | E | 278 | −14.120 | −11.310 | −30.463 | 1.00 | 0.00 |
| 1084 | CG | PHE | E | 278 | −13.890 | −10.246 | −29.392 | 1.00 | 0.00 |
| 1085 | CD1 | PHE | E | 278 | −13.785 | −10.616 | −28.048 | 1.00 | 0.00 |
| 1086 | CD2 | PHE | E | 278 | −13.784 | −8.900 | −29.756 | 1.00 | 0.00 |
| 1087 | CE1 | PHE | E | 278 | −13.563 | −9.643 | −27.070 | 1.00 | 0.00 |
| 1088 | CE2 | PHE | E | 278 | −13.562 | −7.927 | −28.778 | 1.00 | 0.00 |
| 1089 | CZ | PHE | E | 278 | −13.449 | −8.299 | −27.436 | 1.00 | 0.00 |
| 1090 | N | PHE | E | 279 | −15.701 | −13.656 | −29.449 | 1.00 | 0.00 |
| 1091 | CA | PHE | E | 279 | −16.257 | −14.556 | −28.455 | 1.00 | 0.00 |
| 1092 | C | PHE | E | 279 | −15.295 | −15.693 | −28.241 | 1.00 | 0.00 |
| 1093 | O | PHE | E | 279 | −15.007 | −16.424 | −29.175 | 1.00 | 0.00 |
| 1094 | CB | PHE | E | 279 | −17.615 | −15.065 | −28.984 | 1.00 | 0.00 |
| 1095 | CG | PHE | E | 279 | −18.238 | −16.106 | −28.056 | 1.00 | 0.00 |
| 1096 | CD1 | PHE | E | 279 | −18.292 | −15.884 | −26.678 | 1.00 | 0.00 |
| 1097 | CD2 | PHE | E | 279 | −18.763 | −17.285 | −28.593 | 1.00 | 0.00 |
| 1098 | CE1 | PHE | E | 279 | −18.894 | −16.826 | −25.839 | 1.00 | 0.00 |
| 1099 | CE2 | PHE | E | 279 | −19.378 | −18.222 | −27.757 | 1.00 | 0.00 |
| 1100 | CZ | PHE | E | 279 | −19.426 | −18.000 | −26.378 | 1.00 | 0.00 |
| 1101 | N | GLY | E | 280 | −14.795 | −15.837 | −26.997 | 1.00 | 0.00 |
| 1102 | CA | GLY | E | 280 | −13.839 | −16.902 | −26.755 | 1.00 | 0.00 |
| 1103 | C | GLY | E | 280 | −13.851 | −17.355 | −25.322 | 1.00 | 0.00 |
| 1104 | O | GLY | E | 280 | −14.609 | −16.843 | −24.513 | 1.00 | 0.00 |
| 1105 | N | ALA | E | 281 | −12.980 | −18.338 | −25.024 | 1.00 | 0.00 |
| 1106 | CA | ALA | E | 281 | −12.925 | −18.858 | −23.669 | 1.00 | 0.00 |
| 1107 | C | ALA | E | 281 | −11.573 | −19.458 | −23.383 | 1.00 | 0.00 |
| 1108 | O | ALA | E | 281 | −10.723 | −19.512 | −24.258 | 1.00 | 0.00 |
| 1109 | CB | ALA | E | 281 | −14.028 | −19.913 | −23.470 | 1.00 | 0.00 |
| 1110 | N | LEU | E | 282 | −11.382 | −19.911 | −22.129 | 1.00 | 0.00 |
| 1111 | CA | LEU | E | 282 | −10.091 | −20.459 | −21.757 | 1.00 | 0.00 |
| 1112 | C | LEU | E | 282 | −10.264 | −21.459 | −20.647 | 1.00 | 0.00 |
| 1113 | O | LEU | E | 282 | −11.040 | −21.230 | −19.733 | 1.00 | 0.00 |
| 1114 | CB | LEU | E | 282 | −9.172 | −19.303 | −21.309 | 1.00 | 0.00 |
| 1115 | CG | LEU | E | 282 | −7.876 | −19.820 | −20.649 | 1.00 | 0.00 |
| 1116 | CD1 | LEU | E | 282 | −6.977 | −20.500 | −21.698 | 1.00 | 0.00 |
| 1117 | CD2 | LEU | E | 282 | −7.120 | −18.637 | −20.014 | 1.00 | 0.00 |
| 1118 | N | LYS | E | 283 | −9.529 | −22.582 | −20.736 | 1.00 | 0.00 |
| 1119 | CA | LYS | E | 283 | −9.608 | −23.547 | −19.659 | 1.00 | 0.00 |
| 1120 | C | LYS | E | 283 | −8.699 | −23.123 | −18.542 | 1.00 | 0.00 |
| 1121 | O | LYS | E | 283 | −7.539 | −22.823 | −18.779 | 1.00 | 0.00 |
| 1122 | CB | LYS | E | 283 | −9.234 | −24.953 | −20.159 | 1.00 | 0.00 |
| 1123 | CG | LYS | E | 283 | −9.763 | −25.996 | −19.155 | 1.00 | 0.00 |
| 1124 | CD | LYS | E | 283 | −9.667 | −27.401 | −19.771 | 1.00 | 0.00 |
| 1125 | CE | LYS | E | 283 | −10.378 | −28.417 | −18.856 | 1.00 | 0.00 |
| 1126 | NZ | LYS | E | 283 | −9.655 | −28.537 | −17.579 | 1.00 | 0.00 |
| 1127 | N | LEU | E | 284 | −9.250 | −23.090 | −17.315 | 1.00 | 0.00 |
| 1128 | CA | LEU | E | 284 | −8.430 | −22.668 | −16.194 | 1.00 | 0.00 |
| 1129 | C | LEU | E | 284 | −7.623 | −23.818 | −15.665 | 1.00 | 0.00 |
| 1130 | O | LEU | E | 284 | −8.058 | −24.956 | −15.741 | 1.00 | 0.00 |
| 1131 | CB | LEU | E | 284 | −9.313 | −22.072 | −15.081 | 1.00 | 0.00 |
| 1132 | CG | LEU | E | 284 | −10.177 | −20.927 | −15.647 | 1.00 | 0.00 |
| 1133 | CD1 | LEU | E | 284 | −11.154 | −20.435 | −14.564 | 1.00 | 0.00 |
| 1134 | CD2 | LEU | E | 284 | −9.284 | −19.760 | −16.113 | 1.00 | 0.00 |
| 1135 | N | LEU | E | 285 | −6.426 | −23.505 | −15.132 | 1.00 | 0.00 |
| 1136 | CA | LEU | E | 285 | −5.604 | −24.568 | −14.577 | 1.00 | 0.00 |
| 1137 | C | LEU | E | 285 | −6.126 | −24.949 | −13.223 | 1.00 | 0.00 |
| 1138 | O | LEU | E | 285 | −6.291 | −26.115 | −12.896 | 1.00 | 0.00 |
| 1139 | CB | LEU | E | 285 | −4.148 | −24.079 | −14.465 | 1.00 | 0.00 |
| 1140 | CG | LEU | E | 285 | −3.485 | −24.108 | −15.855 | 1.00 | 0.00 |
| 1141 | CD1 | LEU | E | 285 | −2.079 | −23.484 | −15.767 | 1.00 | 0.00 |
| 1142 | CD2 | LEU | E | 285 | −3.366 | −25.559 | −16.361 | 1.00 | 0.00 |
| 1143 | OXT | LEU | E | 285 | −6.409 | −23.944 | −12.402 | 1.00 | 0.00 |

Having now fully described this invention, it will be understood to those of ordinary skill in the art that the same can be performed within a wide and equivalent range of conditions, formulations, and other parameters without affecting the scope of the invention or any embodiment thereof. All patents and publications cited herein are fully incorporated by reference herein in their entirety.

Further, the entire disclosure including the specification, drawings and sequence listing of International Application No. PCT/US02/35661, filed Nov. 7, 2002, and U.S. Provisional Application No. 60/331,049 filed Nov. 7, 2001, are hereby incorporated by reference in their entireties.

LENGTHY TABLE
The patent application contains a lengthy table section. A copy of the table is available in electronic form from the USPTO web site (http://seqdata.uspto.gov/?pageRequest=docDetail&DocID=US20070026500A1) An electronic copy of the table will also be available from the USPTO upon request and payment of the fee set forth in 37 CFR 1.19(b)(3).

What is claimed is:

1. A Neutrokine-alpha protein in crystalline form.

2. The protein of claim 1, wherein said Neutrokine-alpha protein is human Neutrokine-alpha protein.

3. The protein of claim 1, wherein said Neutrokine-alpha protein comprises residues 141-285 of human Neutrokine-alpha.

4. The protein of claim 1, wherein said crystalline form is hexagonal.

5. The protein of claim 1, wherein said crystalline form has space group P6₅ or P6₁.

6. The protein of claim 1, wherein said crystalline form has unit cell dimensions of a, b, and c, wherein a is about 123 Å, b is about 123 Å, and c is about 161 Å.

7. The protein of claim 6, wherein said crystalline form has unit cell dimensions of a, b, and c, wherein a is about 123.58 Å, b is about 123.58 Å, and c is about 161.23 Å.

8. The protein of claim 1, wherein said protein diffracts X-rays to greater than or equal to about 2.5 Å.

9. The protein according to claim 1, wherein said protein that effectively diffracts X-ray for the determination of the atomic coordinates of at least a portion of said Neutrokine-alpha protein to a resolution of better than about 5.0 Å, wherein said crystal has a space group of P6₅ with unit cell dimensions of a, b, and c, wherein a is about 123.58 Å, b is about 123.58 Å, and c is about 161.23 Å; wherein said Neutrokine-alpha protein consists of amino acids 141-285 of human Neutrokine-alpha.

10. A method of preparing a protein according to claim 1, said method comprising
(a) preparing a solution comprising a Neutrokine-alpha protein; and
(b) facilitating said solution to form said protein of claim 1, wherein said facilitating comprises a process selected from the group consisting of hanging drop diffusion, microbatch, sitting drop, or dialysis.

11. The method of claim 10, wherein said solution further comprises Mg²⁺ or Zn²⁺.

12. The method of claim 10, wherein said solution further comprises Mg²⁺.

13. The method of claim 12, wherein said solution further comprises dioxane, and citrate.

14. The method of claim 12 wherein the Neutrokine-alpha protein is at a final concentration of between about 1-30 mg/ml.

15. The method of claim 10, wherein said Neutrokine-alpha protein consists of amino acids 141-285 of human Neutrokine-alpha.

16. The method of claim 10, wherein said process is hanging drop diffusion.

17. The method of claim 10, wherein said crystallization solution comprises about 20 mg/mL of said Neutrokine-alpha protein, about 25 mM citrate, about 125 mM NaCl, about 25% dioxane, about 25 mM MgCl₂ and wherein said solution has a pH of about 6.

18. A method of designing or identifying a biologically active molecule, said method comprising:
(a) providing a model comprising coordinates defining a three-dimensional shape representative a Neutrokine-alpha protein;
(b) designing or identifying said molecule based on said model.

19. The method of claim 18, wherein said a Neutrokine-alpha protein comprises amino acids 158-168, 171-181, 217-223 or 237-243 of hNeutrokine-alpha.

20. The method of claim 18, wherein said Neutrokine-alpha protein comprises amino acids 141-285 of hNeutrokine-alpha.

21. The method of claim 18, wherein said model further comprises one or more of the group consisting of electrostatic potential, lipophilic potential, hydrophilic potential, hydrogen bonding potential, distance parameters, solvent accessible surface, atomic charges, and hydrogen atoms.

22. The method of claim 18, further comprising the step of synthesizing said molecule and testing said molecule for biological activity.

23. The method of claim 22, wherein said molecule mimics or enhances the activity of Neutrokine-alpha.

24. The method of claim 22, wherein said molecule inhibits or reduces the activity of Neutrokine-alpha.

25. The method of claim 18, wherein said Neutrokine-alpha protein consists of amino acids 141-285 of human Neutrokine-alpha, a portion thereof, or a homologue thereof.

26. The method of claim 18, wherein said molecule is structurally and chemically similar to at least a portion of a Neutrokine-alpha protein.

27. The method of claim 26, wherein said portion comprises one or more of the group consisting of β-strand a, β-strand a′, β-strand A, β-strand A′, β-strand B, β-strand B′, β-strand C, β-strand D, β-strand E, β-strand F, β-strand G, β-strand H; the loop between a and a′; the loop between a and A; the loop between A and A″; the loop between A″ and B′; the loop between B′ and B; the loop between B and C; the loop between C and D; the loop between D and E; the loop between E and F; the loop between F and G; and the loop between G and H.

28. The method of claim 27, wherein said portion comprises the loop between D and E.

29. The method of claim 18, wherein said molecule is a peptide.

30. The method of claim 18, wherein said molecule is a peptidomimetic.

31. The method of claim 26, wherein said molecule is a non-peptide.

32. The method of claim 18, wherein said molecule binds to a portion of said Neutrokine-alpha protein.

33. The method of claim 32, wherein said portion comprises Q148, I150, A151, D152, S153, E154, L169, L170, F172, L2001 T202, D203, I270, S271, L272, D273, G274, and D275 of the A monomer; and T190, Y192, A207, G209, H210, L211, Q213, R214, K216, H218, F220, D222, E223, L224, L226, V227, T228, L229, F230, R231, I233, A251, K252, and E254 of the C monomer.

34. The method of claim 32, wherein said portion comprises the loop between a and a′.

35. The method of claim 34, wherein said molecule is a peptide.

36. The method of claim 34, wherein said molecule is a peptidomimetic.

37. The method of claim 34, wherein said molecule is a non-peptide.

38. A computer readable medium having stored thereon a model of a Neutrokine-alpha protein or a portion thereof.

39. The medium of claim 38, wherein said model comprises the coordinates of human Neutrokine-alpha as listed in Table 2.

40. A method of identifying or designing a molecule or molecular fragment that binds to a Neutrokine-alpha protein, said method comprising
(a) providing a computer model of said Neutrokine-alpha protein;
(b) employing a computational method to perform a fitting operation between said computer model of said Neutrokine-alpha protein and a computer model of a molecule or molecular fragment;
(c) analyzing the results of said fitting operation to determine the association between said computer model of said molecule or molecular fragment and said computer model of said Neutrokine-alpha.

41. The method according to claim 40, further comprising synthesizing said molecule and testing said molecule for the ability to inhibit Neutrokine-alpha.

42. The method according to claim 40, wherein said computer model of said Neutrokine-alpha comprises amino acids 158-168, 171-181, 217-223, 237-243, 206-236, 265-275 or 151-275 of human Neutrokine-alpha.

43. The method according to claim 40, wherein said computer model of said Neutrokine-alpha comprises amino acids 141-285 of human Neutrokine-alpha.

44. The method according to claim 40, wherein said molecule or said computer model of said molecule or molecular fragment binds to or fits into a depression, wherein said depression comprises Q148, I150, A151, D152, S153, E154, L169, L170, F172, L200, T202, D203, I270, S271, L272, D273, G274, and D275 of a first hNeutrokine-alpha monomer and T190, Y192, A207, G209, H210, L211, Q213, R214, K216, H218, F220, D222, E223, L224, V227, T228, L229, F230, R231, I233, A251, K252, and E254 of a second monomer of hNeutrokine-alpha.

45. The method according to claim 44, wherein said molecule or said computer model of said molecule or molecular fragment forms one, two or more noncovalent interactions with one or more amino acids selected from the group consisting of D152, S153, E154, F172, T202, D203, S271, D273, D275, Y192, H210, L211, Q213, R214, K216, H218, F220, D222, E223, T228, F230, R231, K252 and E254.

46. The method according to claim 40, wherein said molecule or said computer model of said molecule or molecular fragment binds to or fits into a depression on Neutrokine-alpha, wherein said depression comprises Y201, Q234, N235, N242, S244 and N243 of one monomer of hNeutrokine-alpha.

47. The method according to claim 46, wherein said molecule or said computer model of said molecule or molecular fragment forms one, two or more noncovalent interactions with one or more amino acids selected from the group consisting of Y201, Q234, N235, N242, S244 and N243.

48. The method of claim 40, wherein said molecule or said computer model of said molecule or molecular fragment is designed de novo.

49. The method of claim 40, wherein said molecule or said computer model of said molecule or molecular fragment is selected from a database of compounds.

50. The method of claim 40, wherein said molecule or said computer model of said molecule or molecular fragment is constructed from chemical fragments.

51. The method of claim 40, further comprising
(a) after performing said analyzing step, modifying a portion of said molecule or said computer model of said molecule or molecular fragment;
(b) employing a computational means to perform a fitting operation between said modified molecule or said computer model of said modified molecule or modified molecular fragment and said computer model of said Neutrokine-alpha; and
(c) analyzing the results of said fitting operation to quantify the association between said modified computer model of said compound and said computer model of said Neutrokine-alpha.

52. The method of claim 40, wherein said fitting operation comprises a docking algorithm.

53. The method of claim 52, wherein said docking algorithm comprises a flexible docking process.

54. The method of claim 40, wherein said analyzing step comprises evaluating a free energy of association between said molecule or said computer model of said molecule or molecular fragment and said computer model of said Neutrokine-alpha.

55. The method of claim 40, wherein said analyzing step comprises evaluating a hydropathic interaction.


SEQUENCE LISTING (18 sequences)

SEQ ID NO: 1 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 2 - protein, 152 aa, Homo sapiens
RTPSDKPVAHVVANPQAEGQLQWLNDRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 3 - protein, 144 aa, Homo sapiens
KPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 4 - protein, 163 aa, Homo sapiens
  MISC_FEATURE (15)..(15): Xaa equals Ser; omitted at asterisk in TRAIL sequence depicted in Figure 1
  MISC_FEATURE (16)..(16): Xaa equals Asn; omitted at asterisk in TRAIL sequence depicted in Figure 1
  MISC_FEATURE (17)..(17): Xaa equals Thr; omitted at asterisk in TRAIL sequence depicted in Figure 1
  MISC_FEATURE (18)..(18): Xaa equals Leu; omitted at asterisk in TRAIL sequence depicted in Figure 1
  MISC_FEATURE (19)..(19): Xaa equals Ser; omitted at asterisk in TRAIL sequence depicted in Figure 1
  MISC_FEATURE (20)..(20): Xaa equals Ser; omitted at asterisk in TRAIL sequence depicted in Figure 1
  MISC_FEATURE (21)..(21): Xaa equals Pro; omitted at asterisk in TRAIL sequence depicted in Figure 1
  MISC_FEATURE (22)..(22): Xaa equals Asn; omitted at asterisk in TRAIL sequence depicted in Figure 1
  MISC_FEATURE (23)..(23): Xaa equals Ser omitted at asterisk in TRAIL sequence depicted in Figure 1
PQRVAAHITGTRGRXXXXXXXXXKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 5 - protein, 146 aa, Homo sapiens
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 6 - protein, 155 aa, Mus musculus
QPFAHLTINAASIPSGSHKVTLSSWYHDRGWAKISNMTLSNGKLRVNQDGFYYLYANICFRHHETSGSVPTDYLQLMVYVVKTSIKIPSSHNLMKGGSTKNWSGNSEFHFYSINVGGFFKLRAGEEISIQVSNPSLLDPDQDATYFGAFKVQDID

SEQ ID NO: 7 - protein, 22 aa, Homo sapiens
FTASENHLRHCLSCSKCRKEMG

SEQ ID NO: 8 - protein, 20 aa, Homo sapiens
YSTHWNDLLFCLRCTRCDSG

SEQ ID NO: 9 - protein, 21 aa, Homo sapiens
YTQLWNWVPECLSCGSRCSSD

SEQ ID NO: 10 - protein, 26 aa, Homo sapiens
ECFDLLVRHCVACGLLRTPRPKPAGA

SEQ ID NO: 11 - protein, 26 aa, Homo sapiens
EYFDSLLHACIPCQLRCSSNTPPLTC

SEQ ID NO: 12 - protein, 26 aa, Homo sapiens
KFYDHLLRDCISCASICGQHPKQCAY

SEQ ID NO: 13 - protein, 23 aa, Artificial sequence
  consensus sequence of receptor residues that particpate in specific recognition of neutrokine-alpha
  MISC_FEATURE (2)..(2): Xaa equals any amino acid
  MISC_FEATURE (5)..(5): Xaa equals any amino acid
  MISC_FEATURE (9)..(9): Xaa equals any amino acid
  MISC_FEATURE (11)..(12): Xaa equals any amino acid
  MISC_FEATURE (14)..(14): Xaa equals any amino acid
  MISC_FEATURE (16)..(20): Xaa equals any amino acid
EXFDXLLRXCXXCXLXXXXXPKP

SEQ ID NO: 14 - protein, 16 aa, Homo sapiens
GEDPGTTPGHSVPVPA

SEQ ID NO: 15 - DNA, 38 bp, Artificial Sequence
  Primer
cagactggatccgccaccatggatgactccacagaaag

SEQ ID NO: 16 - DNA, 33 bp, Artificial Sequence
  primer
cagactggtaccgtcctgcgtgcactacatggc

SEQ ID NO: 17 - protein, 309 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 18 - protein, 13 aa, Artificial Sequence
  Cyclic peptide
CRKKVHFGDELSC